(19) United States
(12) Patent Application Publication
(10) Pub. No.: US 2021/0228531 A1
(43) Pub. Date: Jul. 29, 2021

(54) TARGETED TREATMENT OF AUTISM SPECTRUM DISORDER AND OTHER NEUROLOGICAL OR PSYCHIATRIC DISORDERS

(71) Applicants: Tufts Medical Center, Inc., Boston, MA (US); Auttx, LLC, Newton, MA (US)

(72) Inventors: Isabelle Draper, Andover, MA (US); Alan S. Kopin, Wellesley, MA (US); Donna Slonim, Woburn, MA (US)

(21) Appl. No.: 15/734,843

(22) PCT Filed: Jun. 5, 2019
(86) PCT No.: PCT/US19/35655

§ 371 Date: Dec. 3, 2020

Related U.S. Application Data
(60) Provisional application No. 62/681,086, filed on Jun. 5, 2018.

(51) Int. Cl. A61K 31/341; A61K 31/122; A61P 25/28

(52) U.S. Cl. CPC A61K 31/341; A61P 25/28; A61K 31/122

(57) ABSTRACT

Disclosed are methods of treating autism spectrum disorder (ASD) by administering a therapeutically effective amount of an isoprenoid antibiotic to subjects identified with a splicing defect in an ASD associated gene. The method of treating a subject with a neurological disease is carried out by identifying the subject comprising a splicing defect in an autism spectrum disorder (ASD)-associated gene, the target gene being characterized as having an hnRNP L binding site. The subject is treated by administering a spliceopathy rescue agent to repair the splicing defect. Also disclosed are methods of upregulating hnRNP L and hnRNP L targets by administering a therapeutically effective amount of an isoprenoid antibiotic. Methods of screening compounds for use in treating autism spectrum disorder (ASD) are also described.

### RELATED APPLICATIONS

This application claims benefit of and priority to provisional patent application U.S. Ser. No. 62/681,086, filed on Jun. 5, 2018; the contents of which are hereby incorporated by reference in its entirety.

### FIELD OF THE INVENTION

The present invention relates to treatment of neurological disorders, such as autism spectrum disorder (ASD) and intellectual disability.

### BACKGROUND

There is a high incidence of autism spectrum disorder (ASD) in the general population (˜1 in 68 children). Very few therapeutics have effects on the primary symptoms of autism spectrum disorder (and correlated neurological conditions e.g. attention-deficit/hyperactivity disorder (ADHD), epilepsy, mental retardation, intellectual disability), including those typically used for neuropsychiatric disorders. There exists a need for safe compounds for the treatment of autism spectrum disorder.

### SUMMARY OF THE INVENTION

The invention provides compounds, compositions, and methods for the treatment, e.g., reduction of symptoms, of autism spectrum disorder (ASD) as well as other neurological and/or psychological disorders or conditions.

The method includes the steps of identifying a subject having a splicing defect in an autism spectrum disorder (ASD)-associated gene, wherein the target gene is characterized as having an hnRNP L (heterogeneous nuclear ribonucleoprotein L) binding site, and administering to the subject a spliceopathy rescue agent to repair the splicing defect. In this method, the target gene does not include AB11, ACSS2, AGAP3, AGXT2L2, APP, ATP2B1, ATP2B4, BIN1, BPTF/FALZ, C12orf41/KANSL2, C14orf133/VIPAR, DMD, DTNA, E1F2C2, EPB41L2, FMNL2, GARNL1/RALGAPA1, ITSN2, KIAA1217, LRRFIP1, MAPT, MAX, MEF2A, NCAM1, PALLD, PDLIM7, PPP2R5C, PTPN3, RPGR, RRN3, SAD1/BRSK2, SAMD4A, SEMA6D, SLC25A3, SLC39A9, SMTN, SORBS1, STXBP5, SVIL, TPM1, TPM3, TRIM66, TTN, VPS29, XPNPEP1, or ZMYND8 (U.S. Pat. No. 9,662,314, contents of which are hereby incorporated by reference in its entirety). Exemplary neurological and psychiatric disorders include, but are not limited to, autism, autism spectrum disorder, intellectual disability, attention-deficit/hyperactivity disorder (ADHD), dyslexia, epilepsy, bipolar disorder, Alzheimer's disease, Parkinson's disease, depression and schizophrenia.

The splicing defect or spliceopathy may be detected, e.g., using whole genome sequencing and/or identification of aberrant splice variants in a sample of RNA or corresponding cDNA. Examples of such splicing defects or the spliceopathies include, but are not limited to, exon (all or part) skipping, in-frame deletion, exon (all or part) inclusion, intron (all or part) retention, or the usage of cryptic 5′ and 3′ splice sites. Additionally, the splicing defect or the spliceopathy may also include altered relative abundance of alternatively splice variants. For example, the ratio of a predominan brain splice variant vs. a minor brain splice variant may be in an abnormal value/amount. In another example, the ratio of a fetal splice variant vs. an adult splice variant may be in an abnormal value/amount or in an abnormal ratio. In another example, tissue-specific normal variants may be expressed in inappropriate tissues, such as muscle-specific variants expressed in brain. Such non-neuronal splice variants expressed in neuronal tissue indicate an abnormality that is indicative of ASD or another neurological disorder. Detection of the spliceopathies (aberrant splicing) in subject tissues or cells can be achieved using minimally invasive procedures. For example, defects may be detected in the RNA extracted from the patient's peripheral blood lymphocytes, using cDNA-SSCP-HD analysis (see, e.g., Ars et al., Mutations affecting mRNA splicing are the most common molecular defects in patients with neurofibromatosis type 1., Hum Mol Genet. 2000, 22;9(2):237-47).

A spliceopathy rescue agent may be defined as an agent that restores or compensates functional defects caused by splicing defects or spliceopathies. For example, a spliceopathy rescue agent may restore the altered splicing and thus inhibit expression of abnormal mRNA variants or protein isoforms and/or improve expression of normal forms of mRNA or protein. A spliceopathy rescue agent may also restore the tissue specificity, e.g., tissue specific expression, of the target gene. Alternatively, a spliceopathy rescue agent may not directly influence the altered splicing, but compensate a defective function caused by the altered splicing.

Examples of a spliceopathy rescue agent that alters a gene splicing profile include, but are not limited to, those documented in the literature (e.g., Martinez-Montiel et al., Alternative Splicing as a Target for Cancer Treatment, Int. J. Mol. Sci. 2018, 19:545; Bates et al., Pharmacology of Modulators of Alternative Splicing, Pharmacol Rev 2017, 69:63-79, which are incorporated herein by reference in their entirety). Non-limiting exemplary spliceopathy rescue agents include a small molecule, a nucleic acid, an enzyme, a protein, a polypeptide, an antibody or a functional fragment thereof, an aptamer, a RNA-based compound (e.g., a small interfering RNA, a microRNA and a small hairpin RNA), an antisense nucleic acid, a PNA, a CRISPR/Cas construct and the like, whether these are natural or synthetic.

An exemplary small molecule includes ascochlorin, an ascochlorin derivative, or an ascochlorin analogue. An ascochlorin derivative may include a chemical compound derived from ascochlorin as a product of a chemical reaction (e.g., Cylindrol A5, 4-O-methylascochhlorin (MAC)). By comparison, an ascochlorin analog may be structurally similar to ascochlorin. For instance, ascofuranone, an ascofuranone derivative or an ascofuranone analog are non-limiting examples of ascochlorin analogues. Exemplary ascochlorin derivative compounds include an ascochlorin glycoside Vertihemipterin A, a aglycone thereof, 4′,5′-dihydro-4′-hydroxyascochlorin, 8′-hydroxyascochlorin; LL-Z1272delta, 8′,9′-dehydroascochlorin, ascofuranone, ascofuranol, AS-6, Cylindrol A5, 4-O-methylascochhlorin (MAC), or colletochlorin.

Particularly preferred are compounds characterized as having minimal or absence of clinical toxicity. For example, MAC has been tested in clinical trials (see, for example, U.S. Pat. No. 3,995,061, 1976) and was well tolerated. The ascochlorin derivatives 4-O-methyl-ascochlorin (MAC), and 4-O-ethyl-ascochlorin display low toxicity as assessed by high LD50 after IP or oral administration (see, for example, Hosokawa T et al., U.S. Pat. No. 3,995,061, 1976). Suitable compound include 4-O-methylascochlorin (MAC), 4-O-ethylascochlorin, and other derivatives/analogs, including AS-6, ascofuranone (AF) and AF-like analogs/ubiquinol mimics isolated via novel routes of synthesis using structure activity relationships (SAR) (e.g., AF-like analogues 18 and 19, as described in West et al., Eur J Med Chem. 2017 Dec. 1; 141:676-689), ascochlorin glycoside Vertihemipterin A, a aglycone thereof, 4′,5′-dihydro-4′-hydroxyascochlorin, 8′-hydroxyascochlorin; LL-Z1272delta, 8′,9′-dehydroascochlorin. Other suitable compounds include cefacetrile, cefotaxime, ciproflaxin, netilimicine or a fluoroquinolone/quinolone compound (see, for example, Kang et al., J Proteome Res. 2006 October; 5(10):2620-31).

The hnRNP L binding site may be located within an intron, or within the exon, adjacent to a site of alternative splicing of the target ASD-associated gene in a subject having a splicing defect. More specifically, the gene may have an hnRNP L binding site within 5000, 4000, 3000, 2000, 1000, 500, 400, 300, 200, 100 or 50 base pairs of a site of alternative splicing. Further, the gene may have an hnRNP L binding site within 4000, 3000, 2000, 1000, 900, 800, 700, 600, 500, 400, 300, 200, 100 or 50 base pairs of an RBFox1/A2BP1 binding site. RBFox1 is a splicing factor that has been implicated in ASD (Bill, B. et al., Int Rev Neurobiol. 2013, 113: 251-267). RBFox1 is also a candidate target of hnRNP L (see, e.g., Table 2). In another example, the gene may have an hnRNP L binding site within 4000, 3000, 2000, 1000, 900, 800, 700, 600, 500, 400, 300, 200, 100 or 50 base pairs of the binding site of a splicing factor which is partner of hnRNP L in the splicing complex. For example, aberrant splicing of FOX1 targets (due to a mutation in FOX1, leading in turn to ASD) is also rescued by hnRNP L if the targets are downstream of both splicing factors.

Exemplary target genes in which the subject to be treated has a splicing defect include NF1 gene, TSC1 or TSC2 gene. For example, the subject may be from a cohort with neurofibromatosis having a splicing defect in a NF1 gene or a cohort with tuberous sclerosis having a splicing defect in a TSC1 or TSC2 gene. (Smith and Sadee, Synaptic signaling and aberrant RNA splicing in autism spectrum disorders, Frontiers in Neuroscience, 2011). Similarly, cDNA screens of NF-1 patients revealed that splice-site mutations constitute the most common type of mutation (28-50%).

The splicing defect may be in a target gene associated with ASD within the SHANK (SH3 and multiple ankyrin repeat domains 3)/TSC (Tuberous sclerosis)/mTOR (mammalian target of rapamycin)/ERK (Extracellular Receptor Kinase) signaling pathway. The SHANK/TSC/mTOR/ERK signaling pathway is one mechanism for controlling cell survival, differentiation, proliferation, metabolism, and motility in response to extracellular cues. The components of the SHANK/TSC/mTOR/ERK signaling pathway include, but are not limited to, 4E-BP, Akt, Ampakine, AMPAR, APOER2, β-catenin, BDNF, CADPS2, CaMKI, CDCS, CHD8, CNTNAP2, CREB, DRD2, Dv11, elF4E, Engrailed, ERK, FMRP, Frizzled, GABAR, GKAP, GSK-3β, HGF, Homer, IGF-1, IGFR, IL1RAPL1, JNK, K+ channel, MeCP2, MEK, MET, mGluR, mTOR, NF1, NLGN, NMDAR, NRX, OPHN1, OXT, OXTR, PDS-95, PI3K, PICK1, PKA, PKC, PTEN, PTPS, Raf, Ras, Reelin, RhoGAP2, S6K, Scnla, Shank, SynGAP, TM4SF2, TrkB, TSC1, TSC2, VLDLR and Wnt. (Goldani et al., Frontiers in Psychiatry, 2014, 5:100).

Several components of the SHANK/TSC/mTOR/ERK signaling pathway are associated with ASD (FIGS. 2, 8 and 9).

Preferably, exemplary target genes include, but are not limited to, genes bearing hnRNP L binding sites within the SHANK-TSC-mTOR-ERK ASD disease module, e.g., NRXN1, NRXN2, NRXN3, NLGN3, NLGN4X, NLGN4Y, SHANK2, SHANK3, NF1, TSC1, TSC2, MTOR, FMR1, EIF4E, CACNA1C, GRIN1, GRM1, DRD2, MAPK3, GSK3B, GABRB3, SCN1A, MET, HRAS, VLDLR, AKAP9 and CADPS2. The aforementioned splicing defect may be in genes bearing hnRNP L binding sites that also comprise the more focused SHANK-TSC ASD disease module. Examples of such genes include, but are not limited to, CADPS2, NRXN1, NRXN2, NRXN3, NLGN3, NLGN4X, NLGN4Y, SHANK2, SHANK3, NF1, TSC1, TSC2, FMR1, EIF4E, CACNA1C, MTOR, GRIN1 and GRM1.

Alternatively, the subject to be treated has a splicing defect in any ASD-associated genes that is a target of hnRNP L as described herein. For example, the subject may have a splicing defect in the genes in Table 1 that is a target of hnRNP L. Table 1 includes the SFARI (Simons Foundation Autism Research Initiative) list of autism genes (881 genes). SFARI genes may include genes associated with ASD from an evolving database for the autism research community. More particularly, the subject may have a splicing defect in genes listed in Table 2, which lists a subset of SFARI genes that have a high-scoring hnRNP L motif within 500 bp of one of the Castle splice sites (see, for example, Castle, et al., Nature Genetics 40(12):1416-25, 2008) (338 genes). Table 3 includes a subset of SFARI genes that have a very high scoring putative hnRNP L-binding motif within 500 bp of one of the Castle splice sites (152 genes). Genes listed in Table 4 include a subset of SFARI genes with hnRNP L binding sites near splice events specifically observed in autism (78 genes). Genes listed in Table 5 include a subset of genes bearing hnRNP L binding sites within the SHANK-TSC-mTOR-ERK ASD disease module (27 genes). Genes listed in Table 6 include a subset of genes bearing hnRNP L binding sites that also comprise the SHANK-TSC ASD disease module (18 genes). The subject comprises or has a mutation in an hnRNP L target gene which results in spliceopathy.

The subject is characterized as having a clinical diagnosis of ASD. For example, the subject may be diagnosed with (a) social communication and social interactions characterized by deficits in social emotional reciprocity; deficits in non-verbal communication; and deficits in developing, maintaining and understanding relationships; and (b) restricted and repetitive behavior characterized by at least 2 of stereotyped movement or speech; insistence on sameness, routines, rituals; restricted, fixated interests; and atypical sensory reactivity. On the other hand, the subject may be from a cohort with neurofibromatosis and tuberous sclerosis who carry a mutation resulting in spliceopathy of the target gene and carry a neurological clinical diagnosis other than of ASD. Exemplary neurological and psychiatric disorders other than ASD include, but are not limited to, intellectual disability, ADHD, dyslexia, epilepsy, bipolar disorder, Alzheimer's disease, Parkinson's disease, depression and schizophrenia.

The invention also encompasses a method of treating a subject with a neurological disease, which includes the steps of identifying the subject having a splicing defect in an ASD-associated gene, wherein the target gene is characterized as having an hnRNP L binding site, and administering to the subject a spliceopathy rescue agent to repair the splicing defect. Exemplary neurological and psychiatric disorders other than ASD include, but are not limited to, intellectual disability, ADHD, dyslexia, epilepsy, bipolar disorder, Alzheimer's disease, Parkinson's disease, depression and schizophrenia. The spliceopathy rescue agent may be ascochlorin, an ascochlorin derivative, or an ascochlorin analogue, or a non-ascochlorin compound that is neither an ascochlorin derivative nor an ascochlorin analog. Non-limiting examples of a non-ascochlorin compound include a small molecule, peptide, RNA-based compound (e.g., antisense oligonucleotides) and antibody, whether these are natural or synthetic. Other non-ascochlorin compounds also include cefacetrile, cefotaxime, ciproflaxin, netilimicine or a fluoroquinolone/quinolone compound (see, for example, Kang et al., J Proteome Res. 2006 October; 5(10):2620-31). Alternatively, the spliceopathy rescue agent may include a combinational therapy composed of ascochlorin, an ascochlorin derivative, or an ascochlorin analogue and a non-ascochlorin compound.

An exemplary method of treating a subject with a neurological disease may be carried out by identifying the subject having a splicing defect in an ASD-associated gene, wherein the target gene is characterized as having an hnRNP L binding site. The method includes administering to such a subject ascochlorin, an ascochlorin derivative, or an ascochlorin analogue (e.g., ascofuranone, an ascofuranone derivative or an ascofuranone analog) to repair the splicing defect. In this method, as described above, the ASD-associated target gene does not include AB11, ACSS2, AGAP3, AGXT2L2, APP, ATP2B1, ATP2B4, BIN1, BPTF/FALZ, C12orf41/KANSL2, C14orf133/VIPAR, DMD, DTNA, E1F2C2, EPB41L2, FMNL2, GARNL1/RALGAPA1, ITSN2, KIAA1217, LRRFIP1, MAPT, MAX , MEF2A, NCAM1, PALLD, PDLIM7, PPP2R5C, PTPN3, RPGR, RRN3, SAD1/BRSK2, SAMD4A, SEMA6D, SLC25A3, SLC39A9, SMTN, SORBS1, STXBP5, SVIL, TPM1, TPM3, TRIM66, TTN, VPS29, XPNPEP1, or ZMYND8.

As described above, the target gene in which the subject has a splicing defect may be characterized as having an hnRNP L binding site within the intron, or within the exon, adjacent to a site of alternative splicing. More specifically, the gene may have an hnRNP L binding site within 5000, 4000, 3000, 2000, 1000, 500, 400, 300, 200, 100 or 50 base pairs of a site of alternative splicing. Further, the gene may have an hnRNP L binding site within 4000, 3000, 2000, 1000, 900, 800, 700, 600, 500, 400, 300, 200, 100 or 50 base pairs of an RBFox1/A2BP1 binding site. In another example, the gene may have an hnRNP L binding site within 4000, 3000, 2000, 1000, 900, 800, 700, 600, 500, 400, 300, 200, 100 or 50 base pairs of the binding site of a splicing factor which is partner of hnRNP L in a splicing complex. For example, aberrant splicing of FOX1 targets (due to a mutation in FOX1, leading in turn to ASD) is also rescued by hnRNP L if the targets are downstream of both splicing factors.

As described above, exemplary genes in which the subject to be treated has a splicing defect include NF1 gene, TSC1 or TSC2 gene, e.g., a subject with neurofibromatosis having a splicing defect in a NF1 gene or a subject with tuberous sclerosis having a splicing defect in a TSC1 or TSC2 gene. The splicing defect may be in a gene bearing hnRNP L binding sites within the SHANK-TSC-mTOR-ERK ASD disease module, for example, NRXN1, NRXN2, NRXN3, NLGN3, NLGN4X, NLGN4Y, SHANK2, SHANK3, NF1, TSC1, TSC2, MTOR, FMR1, EIF4E, CACNA1C, GRIN1, GRM1, DRD2, MAPK3, GSK3B, GABRB3, SCN1A, MET, HRAS, VLDLR, AKAP9 and CADPS2 or a gene bearing hnRNP L binding sites that also comprise the SHANK-TSC ASD disease module, for example, CADPS2, NRXN1, NRXN2, NRXN3, NLGN3, NLGN4X, NLGN4Y, SHANK2, SHANK3, NF1, TSC1, TSC2, FMR1, EIF4E, CACNA1C, MTOR, GRIN1 and GRM1.

Alternatively, as described above, the subject has a splicing defect in any ASD-associated gene that is a target of hnRNP L as described herein. For example, the subject may have a splicing defect in any SFARI gene (e.g., Table 1) that is a target of hnRNP L. More particularly, the subject may have a splicing defect in SFARI genes with a high-scoring hnRNP L motif within 500 bp of one of the Castle splice sites (e.g., Table 2), SFARI genes with a very high scoring putative hnRNP L-binding motif within 500 bp of one of the Castle splice sites (e.g., Table 3), SFARI genes with hnRNP L binding sites near splice events specifically observed in autism (e.g., Table 4), a subset of Table 1 genes bearing hnRNP L binding sites within the SHANK-TSC-mTOR-ERK ASD disease module (e.g., Table 5), or genes bearing hnRNP L binding sites that comprise the SHANK-TSC ASD disease module (e.g., Table 6). As described above, the subject has a mutation in the target gene which results in spliceopathy.

Such subjects are also clinically diagnosed with ASD, for example, with (a) social communication and social interactions characterized by deficits in social emotional reciprocity; deficits in non-verbal communication; and deficits in developing, maintaining and understanding relationships; and (b) restricted and repetitive behavior characterized by at least 2 of stereotyped movement or speech; insistence on sameness, routines, rituals; restricted, fixated interests; and atypical sensory reactivity. On the other hand, the subject may be from a cohort with neurofibromatosis and tuberous sclerosis who carry a mutation resulting in spliceopathy of the target gene and carry a neurological clinical diagnosis other than of ASD, for example, intellectual disability, ADHD, dyslexia, epilepsy, bipolar disorder, Alzheimer's disease, Parkinson's disease, depression and schizophrenia.

Also within the invention is a method of treating a subject with a neurological disease, which may be carried out by identifying the subject having a splicing defect in an ASD-associated gene, wherein the target gene is characterized as having an hnRNP L binding site, and administering to the subject a non-ascochlorin compound to repair a splicing defect. Examples of a non-ascochlorin compound include, but are not limited to, a non-ascochlorin small molecule, peptide, RNA-based compound (e.g., antisense oligonucleotides) and antibody, whether these compounds are natural or synthetic.

Similar to the aforementioned therapeutic method using ascochlorin, an ascochlorin derivative, or an ascochlorin analogue, the target gene in which the subject has a splicing defect may be characterized as having an hnRNP L binding site within the intron, or within the exon, adjacent to a site of alternative splicing. More specifically, the gene may have an hnRNP L binding site within 5000, 4000, 3000, 2000, 1000, 500, 400, 300, 200, 100 or 50 base pairs of a site of alternative splicing. Further, the gene may have an hnRNP L binding sites within 4000, 3000, 2000, 1000, 900, 800, 700, 600, 500, 400, 300, 200, 100 or 50 base pairs of an RBFox1/A2BP1 binding site. In another example, the gene may have an hnRNP L binding site within 4000, 3000, 2000, 1000, 900, 800, 700, 600, 500, 400, 300, 200, 100 or 50 base pairs of the binding site of a splicing factor which is partner of hnRNP L in the splicing complex. For example, aberrant splicing of FOX1 targets (due to a mutation in FOX1, leading in turn to ASD) is also rescued by hnRNP L if the targets are downstream of both splicing factors.

Again, similar to the aforementioned method using ascochlorin, an ascochlorin derivative, or an ascochlorin analogue, exemplary genes in which the subject to be treated has a splicing defect include NF1 gene, TSC1 or TSC2 gene, such as subjects with neurofibromatosis or tuberous sclerosis, respectively. Alternatively, the target gene may be a gene associated with ASD within the SHANK/TSC/mTOR/ERK signaling pathway, for example, CADPS2, NRXN1, NRXN2, NRXN3, NLGN3, NLGN4X, NLGN4Y, SHANK2, SHANK3, NF1, TSC1, TSC2, FMR1, EIF4E, DRD2, MAPK3, GSK3B, GABRB3, CACNA1C, MTOR and SCN1A. Preferably, the splicing defect may be in a gene bearing hnRNP L binding sites that also comprise the SHANK-TSC ASD disease module, for example, CADPS2, NRXN1, NRXN2, NRXN3, NLGN3, NLGN4X, NLGN4Y, SHANK2, SHANK3, NF1, TSC1, TSC2, FMR1, EIF4E, CACNA1C, MTOR, GRIN1 and GRM1.

Similar to the aforementioned method using ascochlorin, an ascochlorin derivative, or an ascochlorin analogue, the subject has a splicing defect in any ASD-associated gene that is a target of hnRNP L as described herein. For example, the subject may have a splicing defect in any SFARI genes (e.g., Table 1) that is a target of hnRNP L, SFARI genes with a high-scoring hnRNP L motif within 500 bp of one of the Castle splice sites (e.g., Table 2), SFARI genes with a very high scoring putative hnRNP L-binding motif within 500 bp of one of the Castle splice sites (e.g., Table 3), SFARI genes with hnRNP L binding sites near splice events specifically observed in autism (e.g., Table 4), a subset of Table 1 genes bearing hnRNP L binding sites within the SHANK-TSC-mTOR-ERK ASD disease module (e.g., Table 5), or genes bearing hnRNP L binding sites that comprise the SHANK-TSC ASD disease module (e.g., Table 6). The subject has a mutation in the target gene which results in spliceopathy.

Similar to the aforementioned method using ascochlorin, an ascochlorin derivative, or an ascochlorin analogue, the subject is characterized as having a clinical diagnosis of ASD. For example, the subject may be diagnosed with (a) social communication and social interactions characterized by deficits in social emotional reciprocity; deficits in non-verbal communication; and deficits in developing, maintaining and understanding relationships; and (b) restricted and repetitive behavior characterized by at least 2 of stereotyped movement or speech; insistence on sameness, routines, rituals; restricted, fixated interests; and atypical sensory reactivity. On the other hand, the subject may be from a cohort with neurofibromatosis and tuberous sclerosis who carry a mutation resulting in spliceopathy of the target gene and carry a neurological clinical diagnosis other than of ASD, for example, intellectual disability, ADHD, dyslexia, epilepsy, bipolar disorder, Alzheimer's disease, Parkinson's disease, depression and schizophrenia.

In addition to the therapeutic methods described above, the invention also provides a diagnostic method of identifying a subject suffering from or at risk of developing ASD or developing intellectual disability. For example, the diagnostic method may be carried out by detecting a defect in an hnRNP L gene or mRNA or protein in a tissue or a cell of the subject. Protein and nucleic acid sequences useful in such therapeutic methods include: mRNA: Homo sapiens heterogeneous nuclear ribonucleoprotein L (HNRNPL), transcript variant 1, mRNA, 2,129 bp linear mRNA Accession: NM_001533.2 GI: 52632382; Homo sapiens heterogeneous nuclear ribonucleoprotein L (HNRNPL), transcript variant 2, mRNA 1,895 bp linear mRNA, Accession: NM_001005335.1 GI: 52632384. Useful protein/polypeptide sequences include: heterogeneous nuclear ribonucleoprotein L isoform a [Homo sapiens], 589 aa protein, Accession: NP_001524.2 GI: 52632383; heterogeneous nuclear ribonucleoprotein L isoform b [Homo sapiens], 456 aa protein, Accession: NP_001005335.1 GI: 52632385. The nature of DNA/RNA mutation includes, but is not limited to: deletion, insertion, point mutation, missense mutation, sense mutation, single nucleotide polymorphism, splice site mutation, cryptic splice site recruitment, copy number variation [(e.g., D'Angelo D et al., Defining the Effect of the 16p11.2 Duplication on Cognition, Behavior, and Medical Comorbidities, JAMA Psychiatry. 2016 January; 73(1):20-30)]

Intellectual disability (Intellectual developmental disorder) may be diagnosed using the following three-fold diagnostic criteria: (a) deficits in intellectual function, typically as assessed via IQ tests (with a score <70 thought to represent performance more than two standard deviations below the mean); (b) deficits in adaptive functioning in conceptual, social, or practical domains, which are severe enough that ongoing support is needed to function adequately at home, in school, at work, or in the community; and (c) onset of these intellectual and adaptive deficits during the developmental period. For details, see, e.g., the Diagnostic and Statistical Manual of Mental Disorders (DSM-5) (https://dsm.psychiatryonline.org/doi/book/10.1176/appi.books.9780890425596). In addition, there are also suggestions that the cutoff of 70 could be relaxed a little (to, e.g., 75 or 80) if criteria B and C are met. For example, the DSM-5 describes that “For example, a person with an IQ score above 70 may have such severe adaptive behavior problems in social judgment, social understanding, and other areas of adaptive functioning that the person's actual functioning is comparable to that of individuals with a lower IQ score. Thus, clinical judgment is needed in interpreting the results of IQ tests.” For example, if the adaptive functioning deficits are apparent and the IQ is between 70 and 75 or even 80, the criteria for intellectual disability are met.

In this diagnostic method, the defect in the hnRNP L gene or mRNA or protein in the tissue or the cell of the subject may be assessed by detecting an alteration or a change in an hnRNP L level (e.g., RNA or protein or activity level) versus normal, or an alteration or a change in an hnRNP L mRNA variant or an hnRNP L protein isoform in the tissue or the cell of the subject compared to a normal control hnRNP L level. A decrease or an increase of at least 10% compared to a normal control level may indicate that the subject has or is at risk of developing ASD. Assessment includes using minimally invasive procedures, e.g., using DNA from hair, skin cells, saliva, blood, or iPS cell-derived differentiated cells (e.g., neurons). Assessment of differential hnRNP L expression, examples include but are not limited to mRNA levels, e.g., quantitative RT-PCR analysis using hnRNP L-specific primers (e.g., Origene HNRNPL Human qPCR Primer Pair (NM_001533) cat # HP228107); TwistDx™ isothermal nucleic acid amplification technology that enables combination of primers and detection of multiple hnRNP L variants. For determination of protein levels, assays, e.g., Western blot analysis, using a commercially available anti-human hnRNP L antibody (monoclonal, e.g., clone 4D11, or polyclonal) are useful. hnRNP L variants may be characterized by higher or lower activity compared to a normal control level. Suitable reagents include, but are not limited to, a Tagged/flagged/radiolabeled anti-hnRNP L antibody, a Tagged/flagged/radiolabeled short nucleotide sequence that binds hnRNP L (e.g., CACA repeats, or derived from a known hnRNP L RNA target), Tagged/flagged protein partner (e.g., RBFOX1/A2BP1), or short peptide derived-thereof, and Tagged/flagged nucleotide sequence derived from hnRNP L RNA (based on documented autoregulation). Tagged or flagged reagents are those that are labelled with a radioactive compound visually detectable reagent such as a fluorescent compound (whether the reagent is directly labeled, or by using a secondary conjugated (e.g., Alexa, Cy3, Cy5) antibody directed against the reagent), or that can be detected using a colorimetric assay (e.g., ELISA).

The method of identifying a subject suffering from or at risk of developing ASD or developing intellectual disability may optionally include the step of determining an efficacy of a therapeutic treatment, which is carried out by showing partial, e.g., at least 10% or complete restoration of a normal hnRNP L RNA, protein or activity level, or an hnRNP L mRNA variant or an hnRNP L protein isoform expression pattern in the tissue or the cell of the subject, where normal is defined as control values found in a corresponding normal human tissue.

Additional diagnostic methods may be encompassed by the invention. For example, a method of diagnosing a subject with ASD or a risk of developing ASD may include the steps of contacting a tissue or a bodily fluid sample from the subject with an hnRNP L binding agent and a detectable label to form a complex and measuring an amount of the complex.

Another example of a method encompassed by the invention may include a method of monitoring a disease severity or response to treatment of a subject with ASD, which includes the step of measuring an amount of an hnRNP L RNA, protein or activity level in a tissue or a cell of the subject following administration of a medicament. In this method, a change of an amount of an hnRNP L RNA, protein or activity level over time (e.g., an increase or a decrease) indicates that the disease severity is decreasing in response to treatment.

The invention also provides a method of screening to identify a spliceopathy rescue agent. For example, the screening method may be carried out by providing a neuronal cell expressing hnRNP L or an ASD-associated gene containing an hnRNP L binding site, contacting the cell with a candidate compound, and detecting an increase in hnRNP L or a reduction in a splicing defect in the ASD-associated gene. In this screening method, detection of the increase in hnRNP L or the reduction in the splicing defect indicates that the compound may have a spliceopathy rescue activity. The screening method to identify a spliceopathy rescue agent may optionally include a step of identifying a compound that induces partial or at least 10% or complete restoration of a normal hnRNP L RNA, protein or activity level, or an hnRNP L mRNA variant or an hnRNP L protein isoform expression pattern, in a tissue of the subject where normal is defined as control values found in corresponding normal human tissue. By the screening method, for example, a non-ascochlorin compound may be identified as a spliceopathy rescue agent. Exemplary cell lines useful in screening assays include the following cells available from American Type Culture Collection (ATCC): CRL-2825, CRL-2768, CRL-10742, HTB-186, CRL-2927, CRL-2542, CRL-2526, CRL-3035, CRL-2532, CRL-2533, CRL-1721.1, CRL-2535, CRL-2534, CRL-2137, CRL-3234, CRL-2142, and CRL-2149. Additional cell lines include HTS and AK I cell lines (e.g., AK Cell lines: Mammalian/human (normal or diseased) iPS cells-derived neuroprogenitor, neuron, or glia including oligodendrocytes, astrocytes (e.g., GIBCO® Human Neural Stem Cells (hNSCs, embryonic H9-derived), rat fetal neural stem cells, rat glial precursor cells (rGPC); rat adrenal gland phaeochromocytoma PC-12 cell line); primary neurons/glia cultures; immortalized neuronal cell lines (e.g., neuroblastoma cell lines: human SH-SY5Y, human SK-N-AS, hybrid rat/mouse F11; mouse hippocampal neuronal HT-22 cell line).

In addition to the therapeutic, diagnostic or screening methods described above, another aspect of the invention includes compositions useful for the treatment of a subject with a neurological disease. For example, a composition for treating a subject with a neurological disease may contain a spliceopathy rescue agent, wherein the composition repairs a splicing defect in an ASD-associated gene having an hnRNP L binding site. More specifically, an exemplary composition for treating a subject with a neurological disease may contain ascochlorin, an ascochlorin derivative, or an ascochlorin analogue (e.g., ascofuranone, an ascofuranone derivative or an ascofuranone analog). The target ASD-associated gene of the composition for treating a subject with a neurological disease may have an hnRNP L binding site, more particularly, within 5000, 4000, 3000, 2000, 1000, 500, 400, 300, 200, 100 or 50 base pairs of a site of alternative splicing. For these examples of the compositions useful to treat a subject with a neurological disease, the target ASD-associated gene does not include AB11, ACSS2, AGAP3, AGXT2L2, APP, ATP2B1, ATP2B4, BIN1, BPTF/FALZ, C12orf41/KANSL2, C14orf133/VIPAR, DMD, DTNA, E1F2C2, EPB41L2, FMNL2, GARNL1/RALGAPA1, ITSN2, KIAA1217, LRRFIP1, MAPT, MAX , MEF2A, NCAM1, PALLD, PDLIM7, PPP2R5C, PTPN3, RPGR, RRN3, SAD1/BRSK2, SAMD4A, SEMA6D, SLC25A3, SLC39A9, SMTN, SORBS1, STXBPS, SVIL, TPM1, TPM3, TRIM66, TTN, VPS29, XPNPEP1, or ZMYND8.

Another exemplary composition for treating a subject with a neurological disease may contain a non-ascochlorin compound, wherein the composition repairs a splicing defect in an ASD-associated gene having an hnRNP L binding site. Similar to the composition containing ascochlorin, an ascochlorin derivative, or an ascochlorin analogue, the target ASD-associated gene of the composition may have an hnRNP L binding site within 5000, 4000, 3000, 2000, 1000, 500, 400, 300, 200, 100 or 50 base pairs of a site of alternative splicing.

Similar to the therapeutic, diagnostic or screening methods described above, exemplary target genes of the compositions for the treatment of a neurological disease include NF1 gene, TSC1 or TSC2 gene in which the subject to be treated has a splicing defect. Such a subject may be from a cohort with neurofibromatosis having a splicing defect in a NF1 gene or a cohort with tuberous sclerosis having a splicing defect in a TSC1 or TSC2 gene. Alternatively, the target gene may be a gene associated with ASD within the SHANK-TSC-mTOR-ERK ASD disease module, for example, NRXN1, NRXN2, NRXN3, NLGN3, NLGN4X, NLGN4Y, SHANK2, SHANKS, NF1, TSC1, TSC2, MTOR, FMR1, EIF4E, CACNA1C, GRIN1, GRM1, DRD2, MAPK3, GSK3B, GABRB3, SCN1A, MET, HRAS, VLDLR, AKAP9 and CADPS2 or a gene bearing hnRNP L binding sites that also comprise the SHANK-TSC ASD disease module, for example, CADPS2, NRXN1, NRXN2, NRXN3, NLGN3, NLGN4X, NLGN4Y, SHANK2, SHANKS, NF1, TSC1, TSC2, FMR1, EIF4E, CACNA1C, MTOR, GRIN1 and GRM1.

Alternatively, similar to the methods described above, the subject has a splicing defect in any ASD-associated gene that is a target of hnRNP L as described herein. For example, the subject may have a splicing defect in any SFARI genes (e.g., Table 1) that is a target of hnRNP L, SFARI genes with a high-scoring hnRNP L motif within 500 bp of one of the Castle splice sites (e.g., Table 2), SFARI genes with a very high scoring putative hnRNP L-binding motif within 500 bp of one of the Castle splice sites (see, for example, Castle, et al., Nature Genetics 40(12):1416-25, 2008) (e.g., Table 3), SFARI genes with hnRNP L binding sites near splice events specifically observed in autism (e.g., Table 4), a subset of Table 1 genes bearing hnRNP L binding sites within the SHANK-TSC-mTOR-ERK ASD disease module (e.g., Table 5) or genes bearing hnRNP L binding sites that comprise the SHANK-TSC ASD disease module (e.g., Table 6). For other examples, the subject has a mutation in a putative ASD target gene which includes hnRNP L binding sites or a mutation in the target gene which results in spliceopathy.

Again, similar to the methods described above, the subject is characterized as having a clinical diagnosis of ASD. Or, the subject may carry a hnRNP L mutation resulting in spliceopathy of the target gene and carry a neurological clinical diagnosis other than of ASD, for example, intellectual disability, ADHD, dyslexia, epilepsy, bipolar disorder, Alzheimer's disease, Parkinson's disease, depression and schizophrenia.

A further aspect of the invention also includes a pharmaceutical composition for treating a subject with intellectual impairment or a neurological disease as described above. For example, the pharmaceutical composition contains the compositions useful for the treatment of a subject with a neurological disease comprising a spliceopathy rescue agent, or ascochlorin, an ascochlorin derivative, or an ascochlorin analogue, or a non-ascochlorin compound to repair a splicing defect in a target gene as described above, and a pharmaceutically acceptable carrier.

A further aspect of the invention also includes a method of identifying a subject suffering from or at risk of developing ASD or developing intellectual disability comprising detecting a defect in an ASD-associated gene or in the mRNA or protein of the gene in a tissue or a cell of the subject, the ASD-associated gene being characterized as having an hnRNP L binding site. In some embodiments, detecting the defect in the gene or mRNA or protein in the tissue or cell of the subject comprises detecting an alteration or a change in a RNA level or a protein level or an activity level of the ASD-associated gene versus normal levels or an alteration or a change in an mRNA variant or a protein isoform of the ASD-associated gene in the tissue or cell of the subject compared to a normal control level, wherein a decrease or an increase of at least a certain percentage (e.g., 10%, 20%, 25%, 30%, 40%, 50%, 60%, 70%, 80%, 90%, 95%, 99%, 100%, 2-fold, 5-fold, 10-fold, or more) compared to a normal control level indicates that the subject comprises or is at risk of developing ASD or intellectual disability. In some embodiments, the method described herein further comprises determining an efficacy of a therapeutic treatment, wherein the therapeutic treatment is indicated as effective if resulting in partial or at least a certain percentage (e.g., 10%, 20%, 25%, 30%, 40%, 50%, 60%, 70%, 80%, 90%, 95%, 99%, or more), or complete, restoration of a normal RNA level, a normal protein level or a normal activity level of the ASD-associated gene or an mRNA variant or a protein isoform expression pattern of the ASD-associated gene, in the tissue of the subject where normal is defined as control values found in a corresponding normal human tissue.

A further aspect of the invention also includes a method of identifying an ASD-associated gene, characterized as having an hnRNP L binding site, comprising:

i) providing a library of hnRNP L binding sequences;

ii) obtaining a position specific scoring matrix (PSSM) from the library in step i) to produce an 8-mer consensus hnRNP L binding motif; and

iii) screening a library of target genes by sequence alignment to identify at least one ASD-associated gene comprising the consensus hnRNP L binding motif in step ii).

In some embodiments, the 8-mer consensus hnRNP L binding motif comprises a sequence of ACACACAC (SEQ ID NO: 968) or ACATACAC (SEQ ID NO: 969), or any one of related sequences disclosed herein, such as ATACACAC (SEQ ID NO: 970), ATATACAC (SEQ ID NO: 971), ACGCACAC (SEQ ID NO: 972), ACGTACAC (SEQ ID NO: 973), ATGCACAC (SEQ ID NO: 974), ATGTACAC (SEQ ID NO: 975), or X₁X₂X₃X₄X₅CAX₆ (SEQ ID NO: 976), wherein X₁ is A, C, or T, X₂ is C or T, X₃ is A or G, X₄ is C or T, X₅ is A, G, or T, and X₆ is C or T.

In some embodiments, the at least one ASD-associated gene described herein has an hnRNP L binding site

i) within the intron, or within the exon, adjacent to a site of alternative splicing;

ii) within 500 base pairs of a site of alternative splicing;

iii) within 200 base pairs of an RBFox1/A2BP1 binding site; and/or

i) within 200 base pairs of the hnRNP L binding site of a splicing factor which is partner of hnRNP L in the splicing complex.

In some embodiments, the at least one ASD-associated gene described herein has a high (>=6) or very high (>=10) score according to the PSSM representing hnRNP L binding motifs.

A further aspect of the invention also includes a method of diagnosing a subject having an autism spectrum disorder (ASD) or intellectual disability or having a risk of developing an ASD or intellectual disability, comprising:

i) providing a tissue or a bodily fluid sample from the subject;

ii) measuring the expression levels and/or activity of hnRNP L or an ASD-associated gene in the tissue or bodily fluid sample; and

iii) comparing the measured expression levels and/or activity in step ii) to a pre-determined expression levels and/or activity in a normal subject without an ASD or intellectual disability,

wherein a decreased expression levels and/or activity of hnRNP L or the ASD-associated gene in the subject, relative to the pre-determined expression levels and/or activity in the normal subject without an ASD or intellectual disability, indicates that such subject has an ASD or intellectual disability or has a risk of developing an ASD or intellectual disability.

A further aspect of the invention also includes a method of treating a subject having a neurological disease, such as ASD or intellectual disability, comprising:

i) providing a tissue or a bodily fluid sample from the subject;

ii) measuring the expression levels and/or activity of hnRNP L or an ASD-associated gene in the tissue or bodily fluid sample;

iii) comparing the measured expression levels and/or activity in step ii) to a pre-determined expression levels and/or activity in a normal subject without the neurological disease; and

iv) if the measured expression levels and/or activity in step ii) is less than the pre-determined expression levels and/or activity in the normal subject in step iii), providing the subject a pharmaceutically effective amount of an agent to reduce the severity of the neurological disease.

A further aspect of the invention also includes a method of identifying an agent for treating a subject having a neurological disease, such as ASD and intellectual disability, comprising:

i) providing a tissue or a bodily fluid sample from the subject;

ii) measuring the expression levels and/or activity of hnRNP L or an ASD-associated gene in the tissue or bodily fluid sample;

iii) comparing the measured expression levels and/or activity in step ii) to a pre-determined expression levels and/or activity in a normal subject without the neurological disease; and

iv) if the measured expression levels and/or activity in step ii) is less than the pre-determined expression levels and/or activity in the normal subject in step iii), identifying an agent capable of increasing the expression levels and/or activity in step ii) in a cell-based assay.

A further aspect of the invention also includes a method of monitoring severity of a neurological disease, such as ASD and intellectual disability, in a subject, comprising

i) providing a tissue or a bodily fluid sample from the subject;

ii) measuring the expression levels and/or activity of hnRNP L or an ASD-associated gene in the tissue or bodily fluid sample;

iii) repeating the measurement in step ii) over time; and

iv) comparing the measured expression levels and/or activity in step iii) with the measured expression levels and/or activity in step ii);

wherein a reduction of the measured expression levels and/or activity in step iii) relative to the measured expression levels and/or activity in step ii) indicates an increased neurological disease severity.

A further aspect of the invention also includes a method of monitoring response to treatment of an agent in a subject having a neurological disease, such as ASD or intellectual disability, comprising

i) providing a tissue or a bodily fluid sample from the subject;

ii) measuring the expression levels and/or activity of hnRNP L or an ASD-associated gene in the tissue or bodily fluid sample;

iii) comparing the measured expression levels and/or activity in step ii) to a pre-determined expression levels and/or activity in a normal subject without the neurological disease;

iv) if the measured expression levels and/or activity in step ii) is less than the pre-determined expression levels and/or activity in the normal subject in step iii), providing the subject a pharmaceutically effective amount of an agent to reduce the severity of the neurological disease;

v) repeating providing in step i) and measurement in step ii) over time; and

vi) comparing the measured expression levels and/or activity in step v) with the measured expression levels and/or activity in step ii);

wherein an increase of the measured expression levels and/or activity in step v) relative to the measured expression levels and/or activity in step ii) indicates a positive response to treatment.

In some embodiments, the ASD-associated gene described herein comprises at least one of NF1, TSC1, and TSC2.

In some embodiments, the ASD-associated gene has an hnRNP L binding site, preferably an hnRNP L binding site

i) within the intron, or within the exon, adjacent to a site of alternative splicing;

ii) within 500 base pairs of a site of alternative splicing;

iii) within 200 base pairs of an RBFox1/A2BP1 binding site; and/or

iv) within 200 base pairs of the binding site of a splicing factor which is partner of hnRNP L in the splicing complex,

optionally wherein the hnRNP L binding site comprises a sequence of ACACACAC (SEQ ID NO: 968) or ACATACAC (SEQ ID NO: 969), or any one of related sequences disclosed herein, such as ATACACAC (SEQ ID NO: 970), ATATACAC (SEQ ID NO: 971), ACGCACAC (SEQ ID NO: 972), ACGTACAC (SEQ ID NO: 973), ATGCACAC (SEQ ID NO: 974), ATGTACAC (SEQ ID NO: 975), or X₁X₂X₃X₄X₅CAX₆ (SEQ ID NO: 976), wherein X₁ is A, C, or T, X₂ is C or T, X₃ is A or G, X₄ is C or T, X₅ is A, G, or T, and X₆ is C or T.

In some embodiments, the ASD-associated gene does not comprise AB11, ACSS2, AGAP3, AGXT2L2, APP, ATP2B1, ATP2B4, BIN1, BPTF/FALZ, C12orf41/KANSL2, C14orf133/VIPAR, DMD, DTNA, E1F2C2, EPB41L2, FMNL2, GARNL1/RALGAPA1, ITSN2, KIAA1217, LRRFIP1, MAPT, MAX , MEF2A, NCAM1, PALLD, PDLIM7, PPP2R5C, PTPN3, RPGR, RRN3, SAD1/BRSK2, SAMD4A, SEMA6D, SLC25A3, SLC39A9, SMTN, SORBS1, STXBPS, SVIL, TPM1, TPM3, TRIM66, TTN, VPS29, XPNPEP1, or ZMYND8.

In some embodiments, the subject comprises a splicing defect of the ASD-associated gene. For example, the splicing defect is caused by at least one mutation in the hnRNP L or the ASD-associated gene.

In some embodiments, the ASD-associated gene described herein comprises at least one gene listed in Tables 1-4, preferably Table 3 or Table 4.

In some embodiments, the ASD-associated gene described herein comprises at least one gene associated with ASD within the SHANK/TSC/mTOR/ERK signaling pathway. For example, the ASD-associated gene comprises at least one gene listed in Table 5 or Table 6.

Each embodiment disclosed herein is contemplated as being applicable to each of the other disclosed embodiments.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

All references, e.g., journal articles, protein or nucleic acid sequence accession numbers, cited U.S. patents, U.S. patent application publications and PCT patent applications designating the U.S., are hereby incorporated by reference in their entirety.

### DESCRIPTION OF THE DRAWINGS

FIG. 1 is a diagram showing a computational analysis that identified hnRNP L candidate targets in autism genes.

FIG. 2 is a table showing patient cohorts.

FIG. 3 is a table showing design of studies to identify patient-specific therapeutics.

FIG. 4 is a schematic and table showing that selection of ascochlorin derivatives/analogs includes low toxicity compounds.

FIG. 5 is a diagram showing the results of bioinformatics screening and identification of high scoring hnRNP L motifs and rbfox 1 site near exons 26, 27, and 32 of TSC2.

FIG. 6 is a table showing absence of significant toxicity with administration of ascochlorin or derivatives thereof in multiple disease models.

FIG. 7 is a diagram showing the signaling pathways and possible treatments associated with ASD.

FIG. 8 is a diagram showing that major components of the SHANK/TSC-centered ASD disease module are candidate targets of hnRNP L.

FIG. 9 is a diagram showing that candidate targets of hnRNP L (circles) are key members of post-synaptic pathways associated with autism spectrum disorder (ASD).

FIG. 10 is a table of ascochlorin and derivatives thereof and corresponding chemical structures.

FIG. 11 is a table showing that the ascochlorin derivatives 4-O-methyl-ascochlorin (MAC), and 4-O-ethyl-ascochlorin display low toxicity as assessed by high LD50 after i.p. or oral administration.

FIG. 12 is a diagram showing compounds 18 and 19 from West et al. Eur. J. Med. Chem. 2017.

FIG. 13 is a diagram showing ascochlorin derivatives/analogs and corresponding chemical structures.

FIG. 14A and FIG. 14B are diagrams showing a model of pathogenic mutation (αP3A23′ G>A)-induced aberrant exon P3A inclusion that is antagonistically regulated by hnRNP L and an hnRNP L-like paralogue hnRNP LL (see Rahman et al. (2013) Sci Rep. 3:2931). Early spliceosome complex formation on CHRNA I pre-mRNA with alternative exon P3A are schematically shown. Large letters indicate functional binding of splicing factors, whereas small letters represent compromised binding of splicing factors. The sequence of point mutation in exon P3A (αP3A23′ G>A) is underlined. FIG. 14A shows that in the wild-type context, hnRNP L (L) binds to wild-type exon P3A and interacts with PTB through the proline-rich region (RRR), which stabilizes PTB binding to the upstream PPT (YYYY). The hnRNP L-PTB interaction prevents association of U2AF⁶⁵ (65) to PPT and U1 snRNP (U1) to the 5′ splice site. The formation of exon-defined E (EDE) complex is thus impaired, which leads to skipping of exon P3A. FIG. 14B shows that the αP3A23′ G>A mutation switches binding to hnRNP L to hnRNP LL (LL). Lack of PRR in hnRNP LL fails to stabilize PTB binding to the upstream PPT, which allows binding of U1 snRNP (U1) and U2AF⁶⁵ (65) on pre-mRNA. The formation of the exon-defined E (EDE) complex facilitates abnormal inclusion of exon P3A.

FIG. 15A and FIG. 15B are diagrams illustrating how one mutation in an hnRNP L binding site can affect hnRNP L binding, leading in turn to aberrant processing of the target. FIG. 15A shows that the hnRNP L binding site (localized either in the exon, or in the intron, of the hnRNP L target gene/transcript) is normal (i.e., displays wild-type sequence). HnRNP L shows normal affinity for the binding site, leading to normal splicing of the corresponding target. FIG. 15B shows that the hnRNP L binding site bears a mutation (e.g., single nucleotide variant, deletion, insertion). HnRNP L shows decreased affinity for the site, leading to abnormal splicing of the corresponding target. Aberrant splicing of the target may in turn result in neurological diseases, such as ASD and intellectual disability.

FIG. 16 is a diagram showing the amino acid sequence of NRXN1.

FIG. 17 is a diagram showing the amino acid sequence of NRXN2.

FIG. 18 is a diagram showing the amino acid sequence of NRXN3.

FIG. 19 is a diagram showing the amino acid sequence of NLGN3.

FIG. 20 is a diagram showing the amino acid sequence of NLGN4X.

FIG. 21 is a diagram showing the amino acid sequence of NLGN4Y.

FIG. 22 is a diagram showing the amino acid sequence of SHANK2.

FIG. 23 is a diagram showing the amino acid sequence of SHANK3.

FIG. 24 is a diagram showing the amino acid sequence of NF1.

FIG. 25 is a diagram showing the amino acid sequence of TSC1.

FIG. 26 is a diagram showing the amino acid sequence of TSC2.

FIG. 27 is a diagram showing the amino acid sequence of MTOR.

FIG. 28 is a diagram showing the amino acid sequence of FMR1.

FIG. 29 is a diagram showing the amino acid sequence of EIF4E.

FIG. 30 is a diagram showing the amino acid sequence of CACNA1C.

FIG. 31 is a diagram showing the amino acid sequence of GRIN1.

FIG. 32 is a diagram showing the amino acid sequence of GRM1.

FIG. 33 is a diagram showing the amino acid sequence of CADPS2.

### DETAILED DESCRIPTION

A small molecule ascochlorin (and/or derivatives and analogs) is useful as a pharmacological modifier of abnormal splicing. The studies described herein identify genes that may be characterized by a splicing defect that can be rescued by ascochlorin and related compounds. Identification of subjects with such defects (in contrast to other mutations, e.g., deletions or other mutations) is useful to segregate patients suitable for treatment.

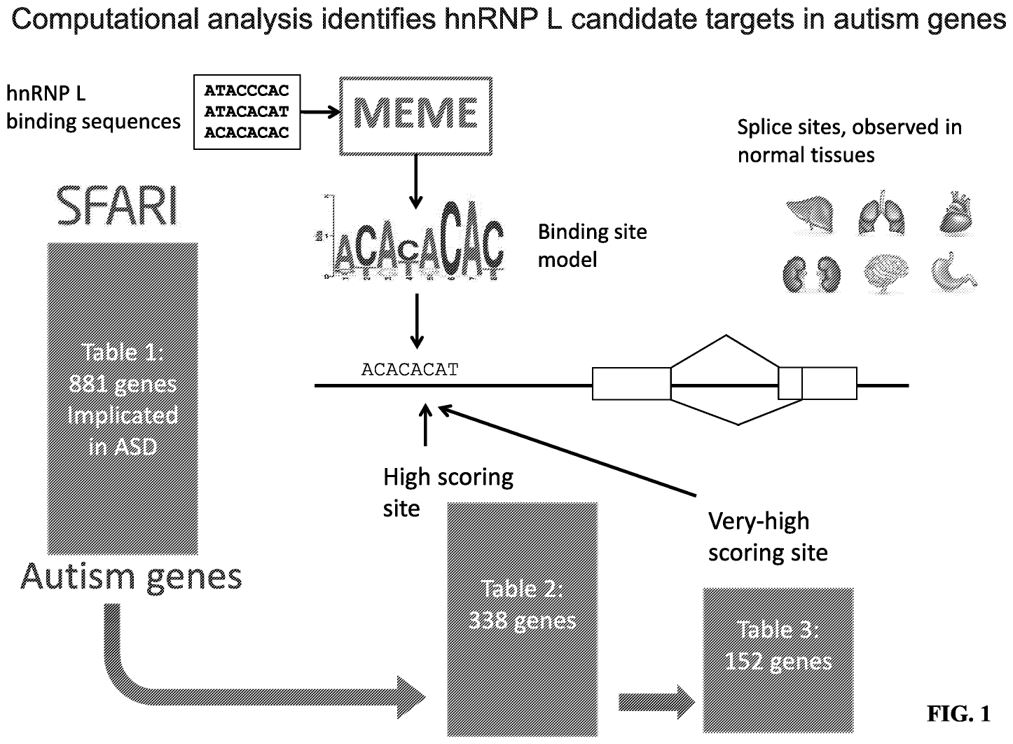

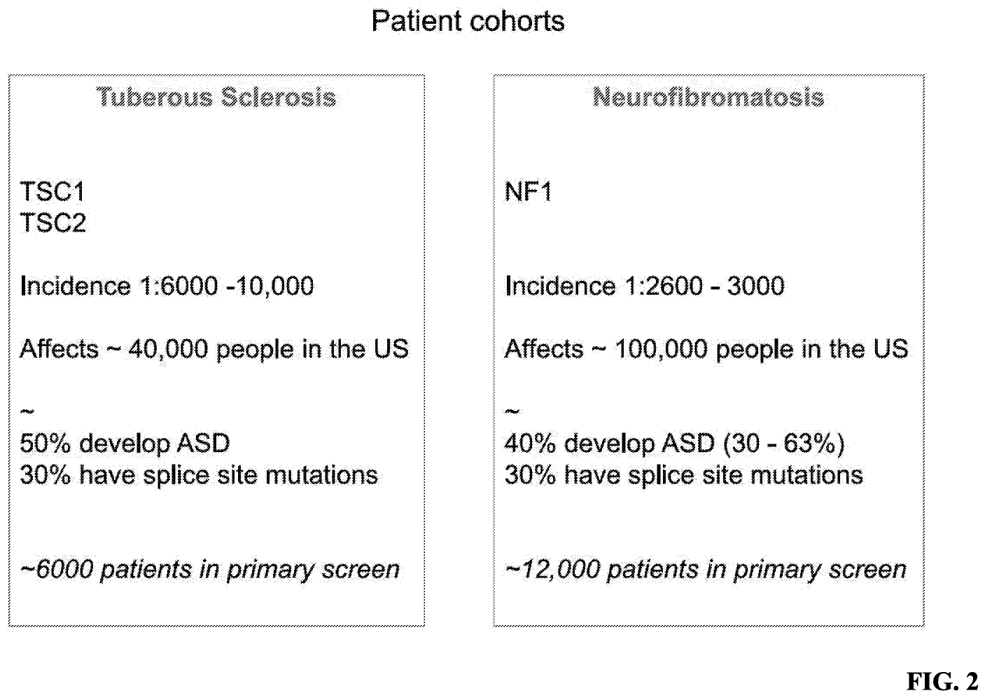

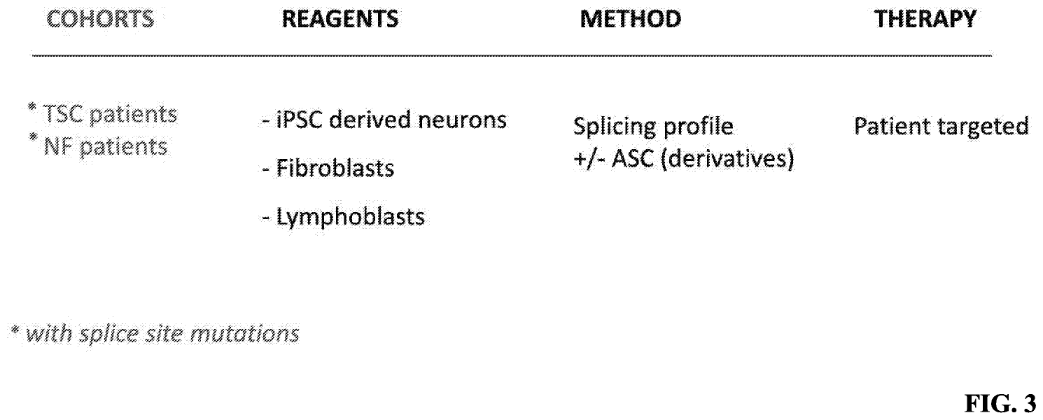

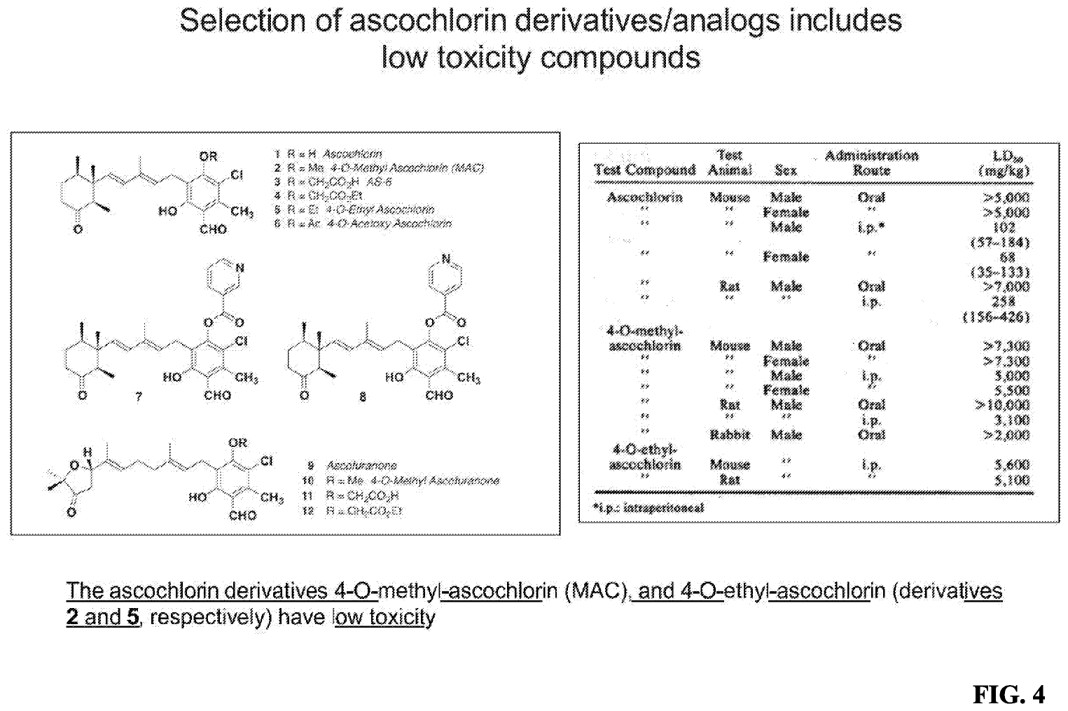

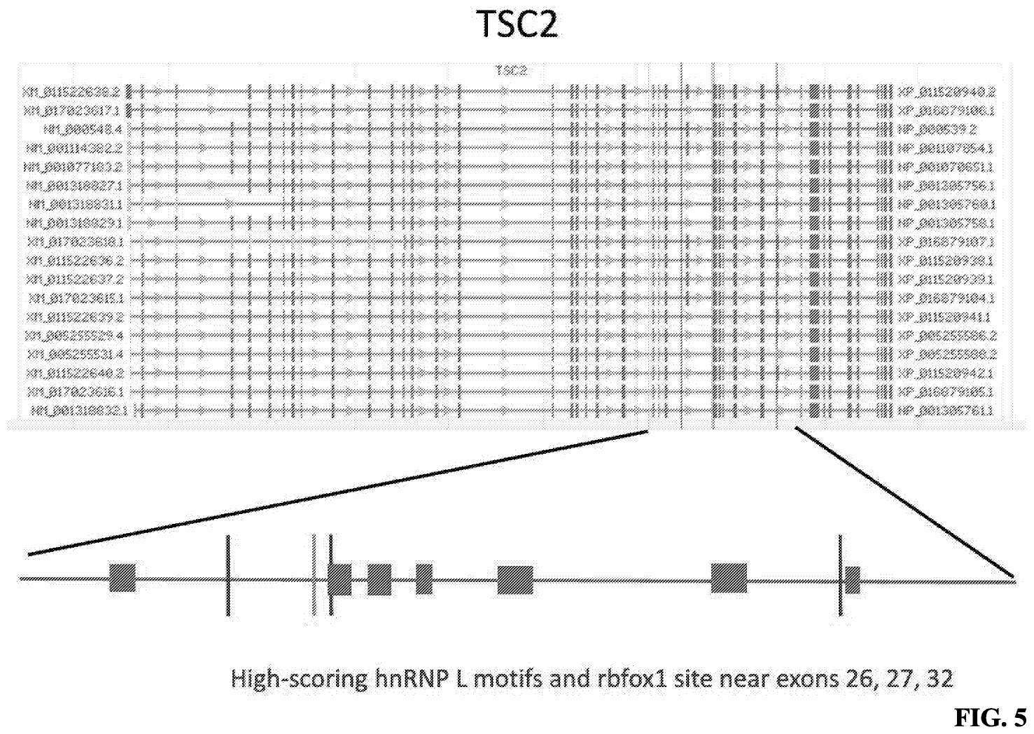

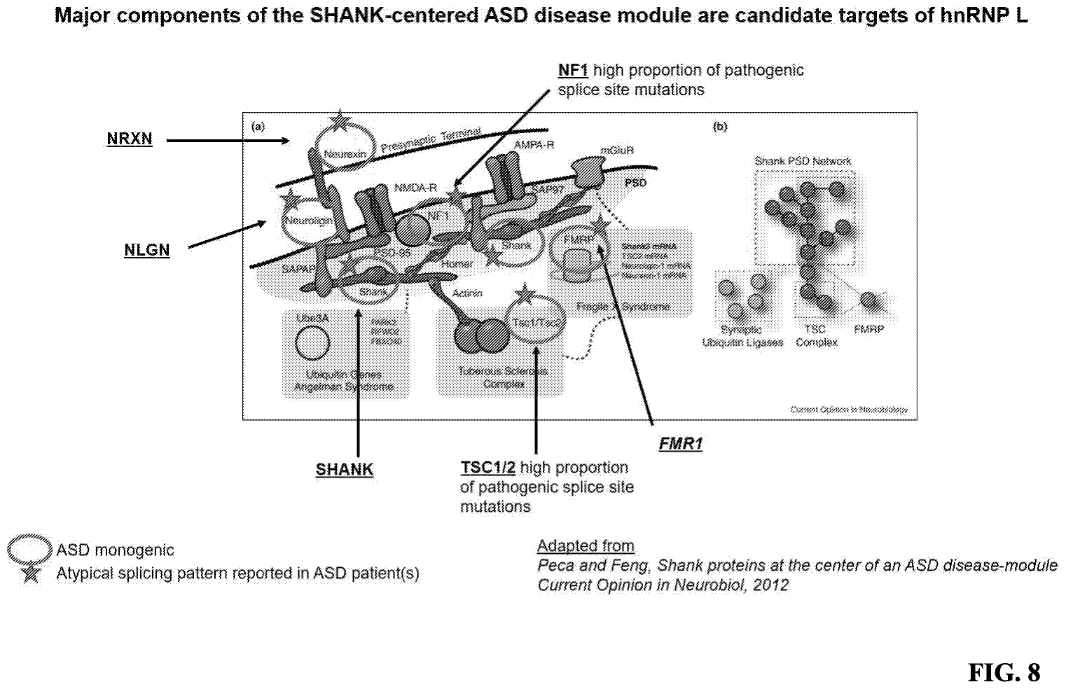

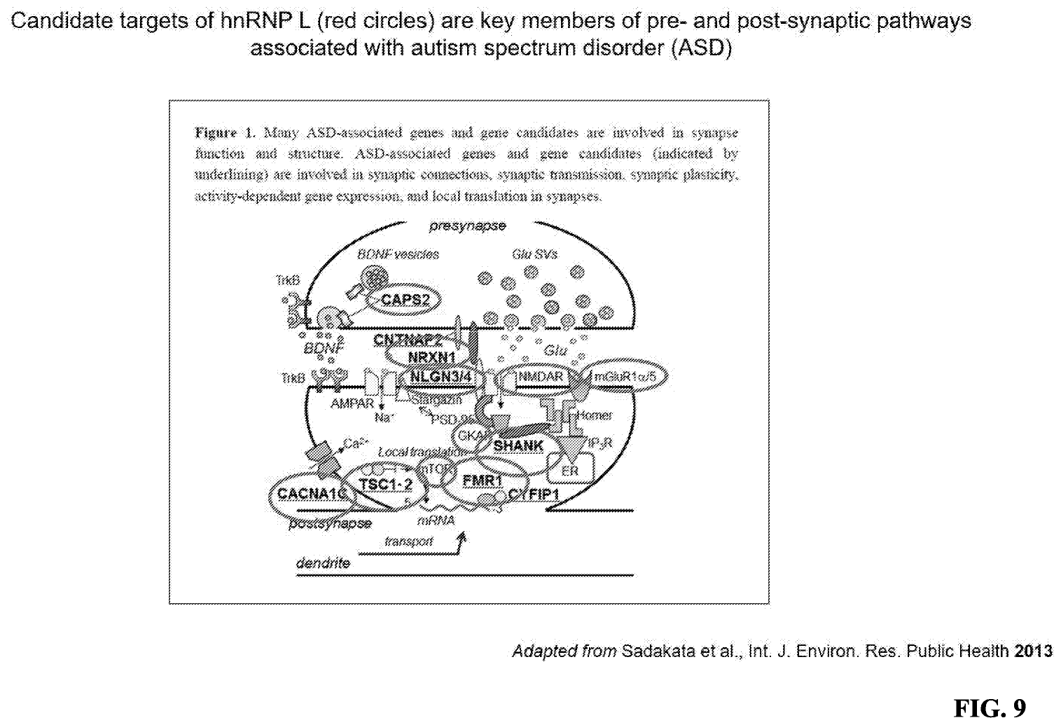

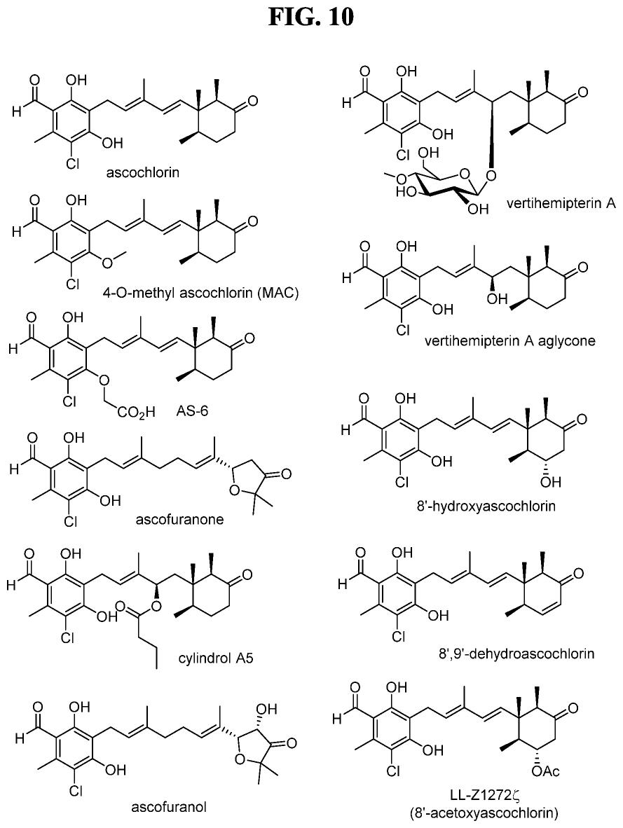

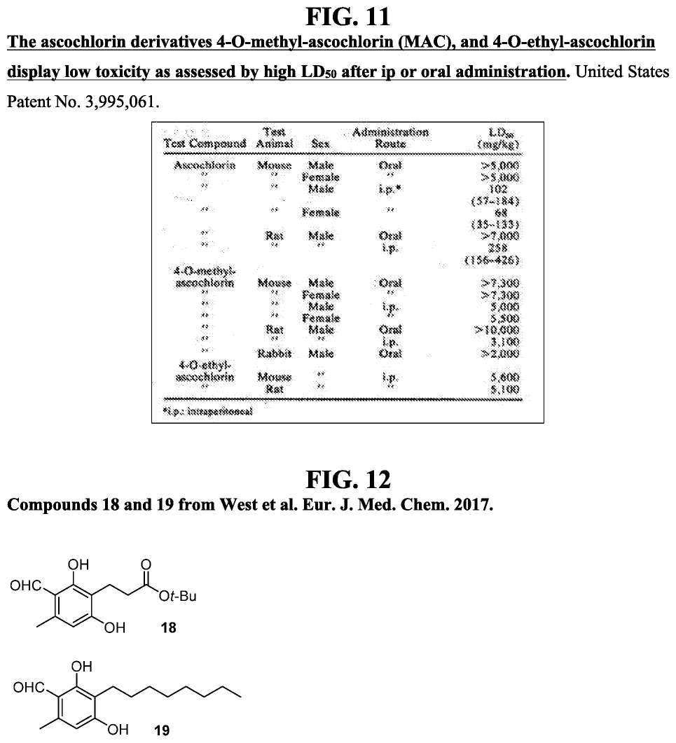

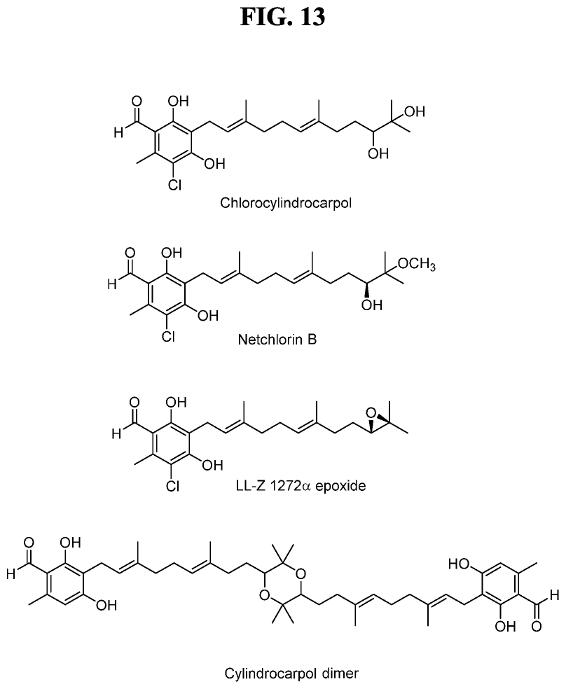

FIG. 1 shows the strategy used to identify hnRNP L candidate targets in autism genes using a binding site model. A bioinformatic approach was established to identify within the publicly available SFARI list of autism genes (Table 1), the targets of hnRNP L. 881 genes involved in autism were examined for hnRNP L binding site(s). Candidate targets downstream of the splicing factor hnRNP L have been identified in the SFARI list of autism genes (Table 1); corresponding target genes have been grouped into genes with high-scoring, likely hnRNP L binding sites (Table 2; 338 genes) and genes with very high-scoring, likely hnRNP L binding sites (Table 3; 152 genes). Among the genes bearing very high scoring hnRNP L binding sites (Table 3), five were found within the SHANK/TSC-centered ASD-disease module of 14 closely interacting partners (module described in Peca and Feng, Curr Opin Neurobiol. 2012 October; 22(5):866-72) (FIG. 8). Among the genes bearing high scoring hnRNP L binding sites (Table 2), multiple (18) are found within the SHANK/TSC/mTOR/ERK signaling pathway associated with ASD (pathways described in Goldani et al., Front Psychiatry. 2014 Aug. 12; 5:100). Targets with nearby (with 500 bases of a hnRNP L binding site) RBFox1/A2BP1 binding sites were also identified.

Of patients with Tuberous Sclerosis (TSC1 or TSC2) or Neurofibromatosis (NF1), approximately ½ also comprise ASD. 30-40% of such patients are characterized with splicing defects.

### Isoprenoid Antibiotics

Isoprenoid antibiotics, including but not limited to the compounds ascochlorin, and its derivatives/analogues (i.e. natural and synthetic related compounds, e.g. ascofuranone, ascofuranol, MAC, AS-6, cylindrol A5, vertihemipterin A, vertihemipterin A aglycone, 8′-hydroxyascochlorin, 8′,9′-dehydroaschchlorin, 8′-acetoxyascochlorin, colletochlorin) can be used directly, and/or as chemical template structures, to treat autism, autism spectrum disorder and related neurological and psychiatric disorders, including but not limited to, mental retardation, learning disability, attention deficit hyperactivity disorder, dyslexia, epilepsy, bipolar disorder, and schizophrenia.

Ascochlorin been shown to increase hnRNP L protein levels in vitro (Kang et al., J Proteome Res. 2006 October; 5(10):2620-3). As described herein, the hnRNP L pathway was utilized to identify novel genes/targets relevant to the treatment of autism spectrum disorder. A cell-based assay to identify drugs that modulate hnRNP L levels is outlined. This cell-based assay, optimized in cell types where hnRNP L plays a role in cell physiology/morphology (including but not limited to, neurons, glia, stem cells, pluripotent/multipotent progenitor cells, or undifferentiated cells), enables the identification of pharmacological compounds that are useful for the development of autism spectrum disorder therapeutics. In addition to ASD, this screening strategy is useful to identify novel targets/pre-therapeutic leads for other neurological and psychiatric disorders, including but not limited to, mental retardation, intellectual disability, learning disability, attention deficit hyperactivity disorder, dyslexia, epilepsy, bipolar disorder, and schizophrenia.

### Autism Spectrum Disorder

Autism spectrum disorder (ASD) is a complex neurodevelopmental disorder that is typically recognized in early childhood and has a lifelong course (Lacivita et al., J. Med. Chem. 2017, 60 (22), 9114-9141 and references cited therein). According to an art-recognized diagnostic criteria, it is characterized by two core symptoms: (1) persistent deficits in social communication and social interaction, (2) restricted, repetitive patterns of behavior, interests, and activities. The diagnosis is based on clinical observation and further established by standardized testing of the patient with the Autism Diagnostic Observation Schedule 2, and/or by parental interview with the Autism Diagnostic Interview-Revised. Thus far, no behavioral, neuroimaging, electrophysiological, or genetic tests can specifically diagnose ASD. Comorbid conditions such as intellectual disability, seizures, and sleep problems are frequent, whereas anxiety, depression, and obsessive-compulsive disorder (OCD) are less frequent.

ASD is distinguished from most other behavioral disorders. The designation of ASD refers to a set of neurodevelopmental disorders comprising an early onset in life and gender prevalence. For example, the prevalence of autism spectrum disorder (ASD) in the United States is 1 in 68 children (1 in 42 boys and 1 in 189 girls) ASD has recently emerged as a major public health issue worldwide.

Altered neurodevelopment during the first and second trimesters of prenatal life is believed to be an underlying neuropathological cause of ASD. Post-mortem studies have unveiled neuroanatomic and cytoarchitectonic aberrations in various brain regions, including cerebellum, hippocampus, inferior olivary complex, amygdala, entorhinal cortex, fusiform gyrus, and anterior and posterior cingulate cortex, with increased growth of the frontal lobes, thinner cortical minicolumns, and increased dendritic spine density.

These aberrations are related to alterations occurring during early pregnancy, such as reduced programmed cell death and/or increased cell proliferation, altered cell migration, abnormal cell differentiation with reduced neuronal body size, abnormal neurite sprouting, and pruning that cause atypical wiring into the brain. In addition, because neurodevelopmental processes are still active into late prenatal and postnatal life, aberrations involve reduced synapse formation and delayed myelination. The observed abnormal neuronal wiring was previously thought to be characterized by long-range hypoconnectivity and local hyperconnectivity. Studies have instead shown that abnormal neuronal wiring is characterized by an individualized combination of hyper- and hypoconnectivity specific to each ASD patient. The plasticity of the brain post-natally and well into adolescence provides an opportunity for therapeutic intervention.

The neurocognitive phenotype of ASD is the result of a complex and an heterogeneous set of genetic and environmental causes. In some patients, the disorder is the result of genetic causes due to known chromosomal aberrations or mutations, while in other patients, the disorder is more likely related to environmental causes, such as prenatal exposure to chemical pollutants, toxins, viruses, or drugs.

Neurological disorders characterized by an hnRNP L binding site aberration-mediated spliceopathy are treated using isoprenoid (prenyl-phenol) antibiotics, including but not limited to the compounds ascochlorin, its derivatives and analogs (e.g. ascofuranone, ascofuranol, MAC, AS-6, cylindrol As, vertihemipterin A, vertihemipterin A aglycone, 8′-hydroxyascochlorin, 8′,9′-dehydroaschchlorin, 8′-acetoxyascochlorin, colletochlorin) which can be used directly, and/or as chemical template structures, to help treat neurological disorders in humans. The relevant neurological and psychiatric disorders include, but are not limited to, autism, autism spectrum disorder, mental retardation, learning disability, intellectual disability, attention deficit hyperactivity disorder, dyslexia, epilepsy, bipolar disorder, and schizophrenia.

### Natural Sources of the Isoprenoid Antibiotic Compounds

Isoprenoid antibiotics were originally isolated from the phytopathogenic fungus Ascochyta viciae. (Sasaki, H. et al. J Antibiot (Tokyo), 1973, 26:676-680). Among them, ascochlorin and ascofuranone have been shown to be non-toxic compounds. Structurally related compounds have been subsequently isolated from other fungi (e.g., Fusarium, Cylindrocladium, Cylindrocladium ilicicola, Nectria coccinea, Colletotrichum nicotianae, Acremonium luzulae, Cephalosporium diospyri, Verticillium, Cylindrocarpon lucidum, Nigrosabulum globosum, and the insect pathogenic fungus Verticillium hemipterigenum). (Hosono, K. et al. J Antibiot (Tokyo), 2009, 62:571-574; Seephonkai, P. et al. J Antibiot (Tokyo), 2004, 57:10-16).

### Physiological Properties of the Isoprenoid Antibiotic Compounds

Studies have demonstrated that the methylated derivative of ascochlorin, 4-O-methylascochlorin (MAC), increases the expression of vascular endothelial growth factor (VEGF) and glucose transporter 1 (GLUT-1) (Jeong J. H. et al. Biochem Biophys Res Commun. 2011;406:353-358). Both VEGF and GLUT-1 RNAs are well-established targets of hnRNP L (Hamilton B. J. et al. Biochem Biophys Res Commun. 1999;261:646-651; Ray P. S. et al. Nature. 2009;457:915-919; Shih S. C. et al. J Biol Chem. 1999;274:1359-1365).

Ascochlorin and/or its derivatives promote the maintenance of normal brain physiology by targeting hnRNP L and/or components of the coordinated hnRNP L-regulated pathway(s). The compounds and methods of the invention provide pharmacological leads to help treat autism spectrum disorder and additional neurological and psychiatric disorders. Ascochlorin and derivatives (e.g., MAC) as well as analogs (e.g., ascofuranone) display antitumorigenic properties, both in vitro and in vivo (summarized in Table 1 in Min-Wen et al., Adv Protein Chem Struct Biol. 2017;108:199-225).

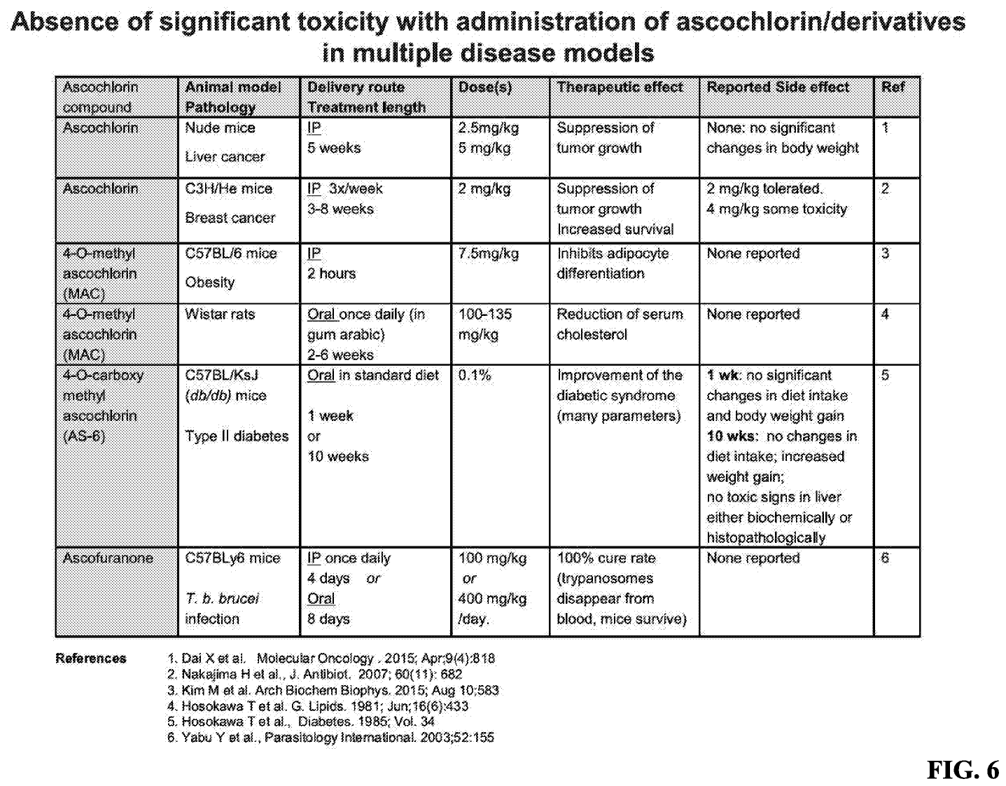

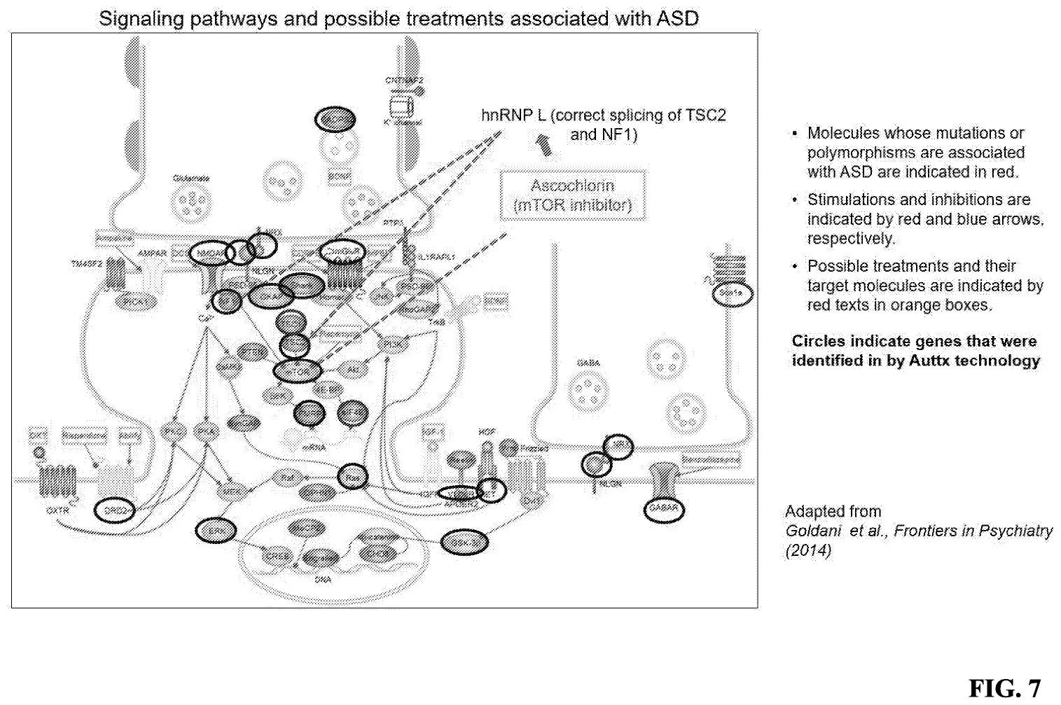

In addition to anticancer properties, ascochlorin and its derivatives exhibit additional physiological activities, including antimicrobial/antiviral activity, trypanocidal properties, hypolipidemic activity, suppression of hypertension, improvement of type I and II diabetes, anti inflammatory, and immunomodulation. (Yabu, Y. et al. Parasitol Int. 2003, 52:155-164; Hosono, K. et al. J Antibiot (Tokyo), 2009, 62:571-574; Lee et al., J Cell Biochem. 2016 April; 117(4):978-87; Shen et al., Eur J Pharmacol. 2016 Nov. 15; 791:205-212). Examples of ascochlorin/derivative treatment effects in various rodent models of disease are also shown in FIG. 6.

Other examples of ascochlorin derivatives may be found in:
(i) Pubchem databases: http://www.ncbi.nlm.nih.gov/pccompound/?term=ascochlorin; by using the “similar compounds” interactive link;
(ii) Pharmaceutical composition and method of using the same. U.S. Pat. No. 3,995,061.
(iii) Ascochlorin derivatives, and pharmaceutical composition containing the same. U.S. Pat. No. 4,500,544;
(iv) Pyridyl carbonyl ascochlorin derivatives and pharmaceutical compositions containing the same. U.S. Pat. No. 4,542,143;
(v) Ligands of nuclear receptor. U.S. Pat. No. 6,605,639.
(vi) Ascochlorin derivative and use thereof as ampk activator. WO 2017/119515.
(vii). R. A. West, et al., African trypanosomiasis: Synthesis & SAR enabling novel drug discovery of ubiquinol mimics for trypanosome alternative oxidase, European Journal of Medicinal Chemistry 141 (2017) 676-689, https://doi.org/10.1016/j.ejmech.2017.09.067.
(viii). Togashi, M et al., Ascochlorin Derivatives as Ligands for Nuclear Hormone Receptors. J. Med. Chem. 2003; 46:4113-4123.
(ix). Saimoto, H et al., Pharmacophore Identification of Ascofuranone, Potent Inhibitor of Cyanide-Insensitive Alternative Oxidase of Trypanosoma brucei. J. Biochem. 2013; 153:267-273.

Additional examples of ascochlorin derivatives include an ascochlorin derivative from Cylindrocarpon sp. FKI-4602. Kawaguchi et al., J Antibiot (Tokyo). 2013 January; 66(1):23-9; ascochlorin derivatives from the leafhopper pathogenic fungus Microcera sp. BCC 17074. Isaka et al., J Antibiot (Tokyo). 2015 January; 68(1):47-5; and competitive Hdhodh inhibitorsm Shen et al., Eur J Pharmacol. 2016 Nov. 15; 791:205-212. The contents of each of the foregoing references is hereby incorporated by reference.

Splicing Factor hnRNP L and Downstream RNA Targets:

Proteome analysis has demonstrated that ascochlorin treatment of human osteosarcoma cells (U2OS) results in a ≥10 fold increase in the levels of three proteins, including the splicing factor hnRNP L (first most upregulated protein, 12x), as well as BIN1 (third most upregulated protein, 10x) (Kang J. H. et al. J Proteome Res. 2006;5:2620-2631). It has been determined by bioinformatics analysis that BIN1 is a candidate target of hnRNP L (Table 2). Importantly, candidate targets comprise a cluster of genes/proteins (i.e., NLGN, NRXN, SHANK, TSC2, FMR1, that are close interacting partners in the SHANK-centered ASD-disease module, as described in Peca and Feng, Curr Opin Neurobiol. 2012 October; 22(5):866-72). Missplicing in these targets has been linked to ASD (Smith R M, Sadee W. Front Synaptic Neurosci. 2011 Jan. 26; 3:1; Talebizadeh et al., J Med Genet 2006;43:e21; Tyburczy et al., PLoS Genet. 2015 Nov. 5; 11(11); Leblond et al., PLoS Genet. 2012 Feburay; 8(2):e1002521; Shinahara et al., J Med Invest. 2004 February; 51(1-2):52-8). In addition, the BIN1 gene was shown to be associated with autism spectrum disorder (Connolly J. J. et al. Child Dev. 2013 January-February; 84(1):17-33). Previous studies have also shown that a mutation in BIN1 is associated with delayed motor and speech development and mild mental retardation (Claeys K. G., et al. Neurology 2010; 74:519-521) as well as other pathologies (Claeys K. G., et al. Neurology 2010; 74:519-521; Fugier C., et al. Nat. Med. 2011; 17:720-725; Toussaint A. et al. Acta Neuropathol. 2011; 121:253-266).

Mutations in the binding sites for splicing factors have been identified that lead to disease in human subjects (whether the binding site is intronic or exonic on the nucleic acid target). For example, the prevalent c. 639+919 G>A mutation in the lysosomal alpha-galactosidase A gene causes Fabry disease in humans by abolishing the binding of the splicing factors hnRNPA1 and hnRNP A2/B1 to a splicing silencer (Palhais B, Dembic M, Sabaratnam R, Nielsen K S, Doktor T K, Bruun G H, Andresen B S. The prevalent deep intronic c. 639+919 G>A GLA mutation causes pseudoexon activation and Fabry disease by abolishing the binding of hnRNPA1 and hnRNP A2/B1 to a splicing silencer, Mol Genet Metab. 2016 November; 119(3):258-269. doi: 10.1016/j.ymgme.2016.08.007. Epub 2016 Aug. 27).

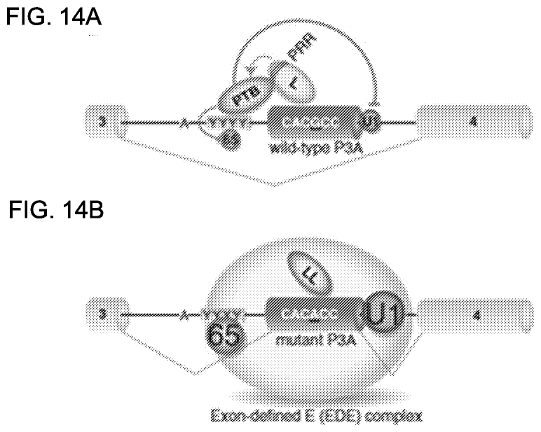

Mutations in the binding site for hnRNP L have also been identified that underlie human disease. A single nucleotide mutation (i.e., αP3A23′ G>A) in exon P3A in the CHRNA1 gene, that encodes the muscle nicotinic acetylcholine receptor alpha subunit (Entrez Gene: 1134 Ensembl: ENSG00000138435), causes severe congenital myasthenic syndrome. The mutation diminishes the affinity of hnRNP L for the corresponding binding sequence on the CHRNA1 transcript. The mechanistic details of the molecular defect are shown in FIGS. 14A and 14B, adapted from Rahman et al. (2013) HnRNP L and hnRNP LL antagonistically modulate PTB-mediated splicing suppression of CHRNA1 pre-mRNA, Sci Rep. 3:2931. doi: 10.1038/srep02931.

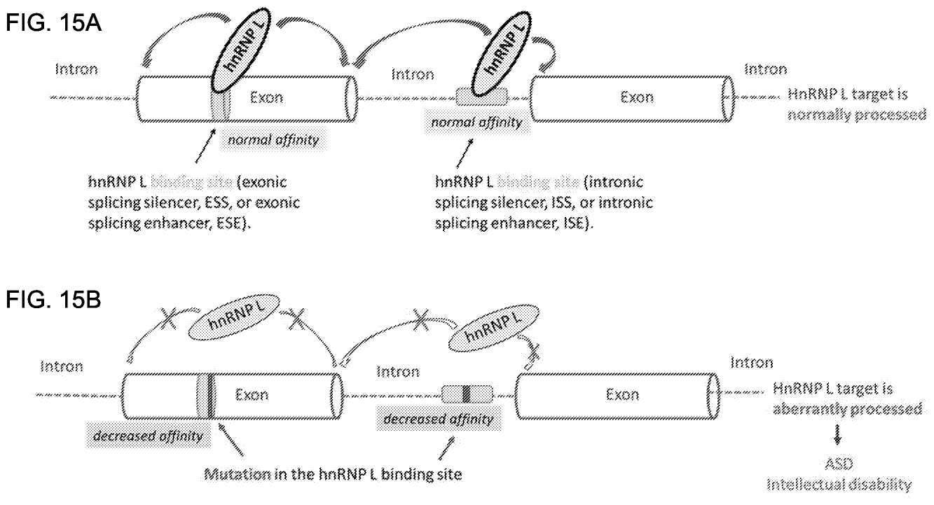

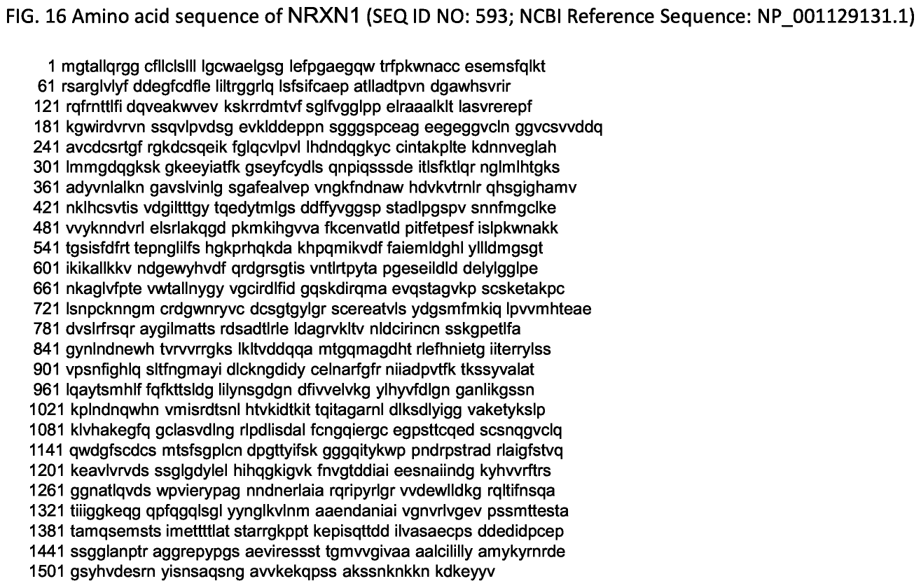

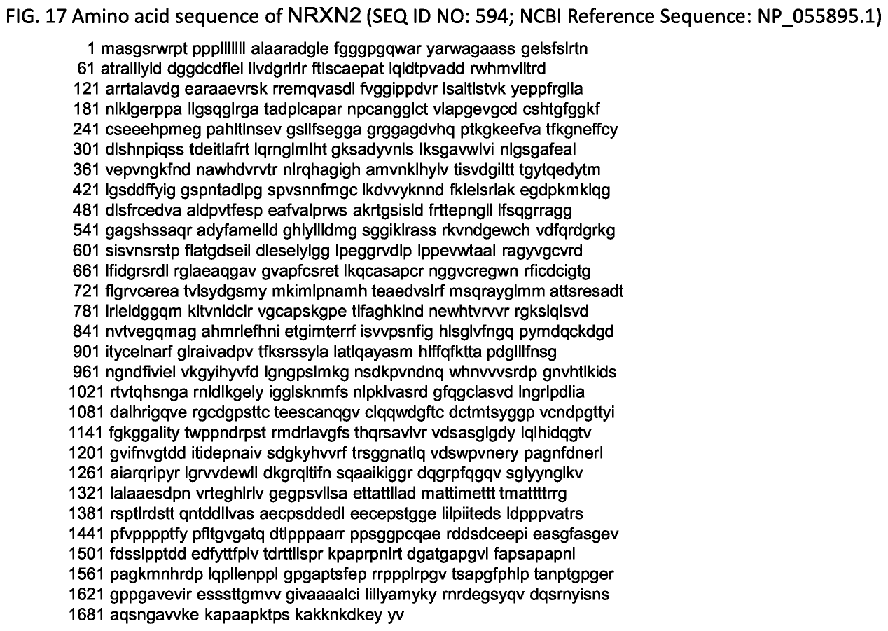

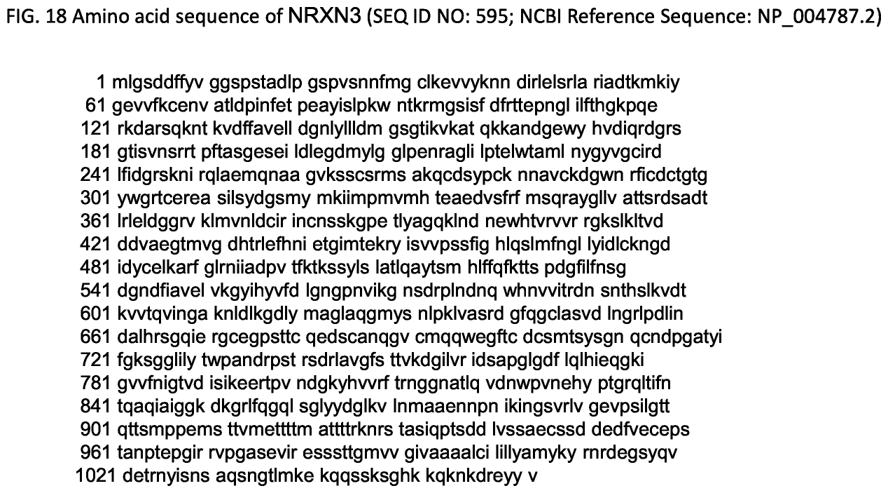

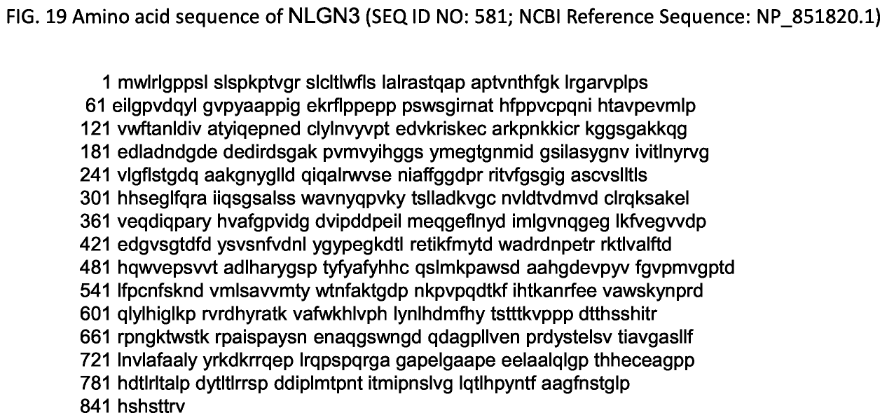

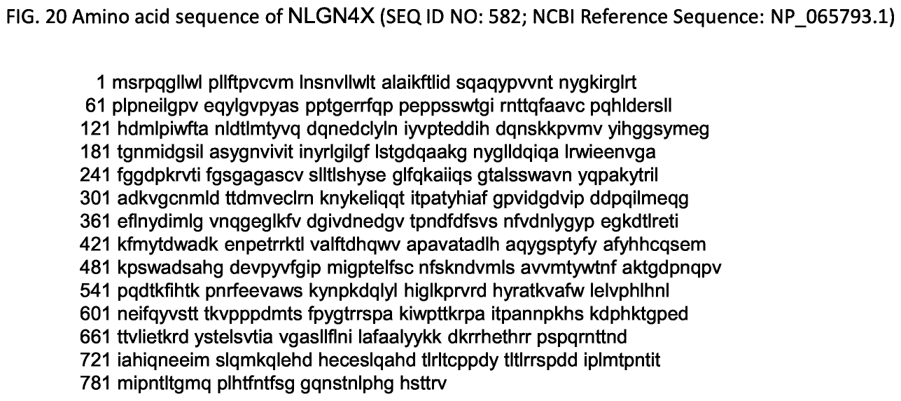

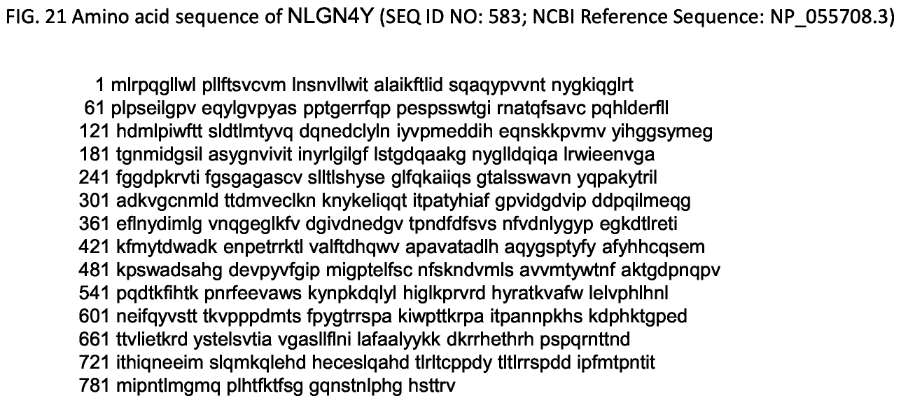

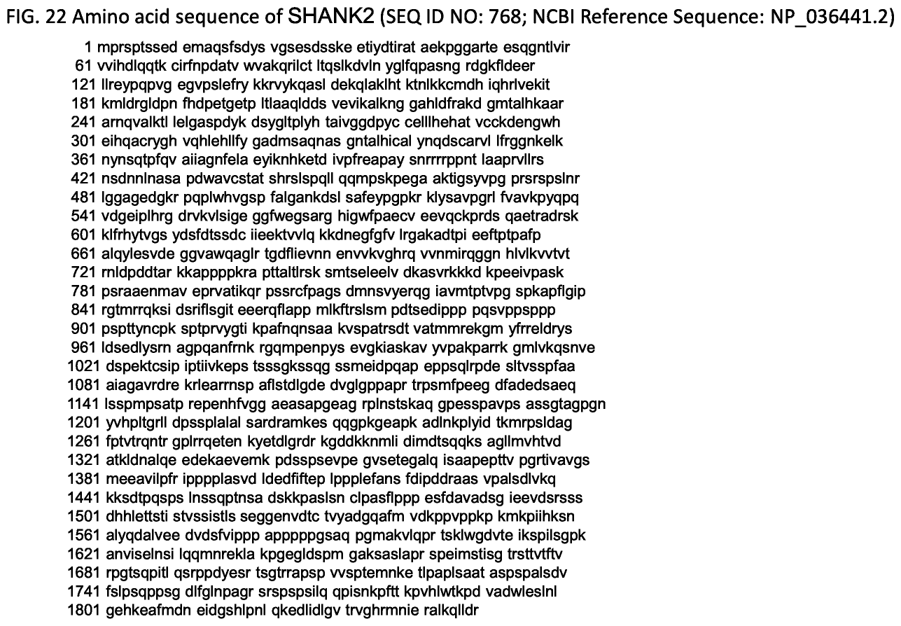

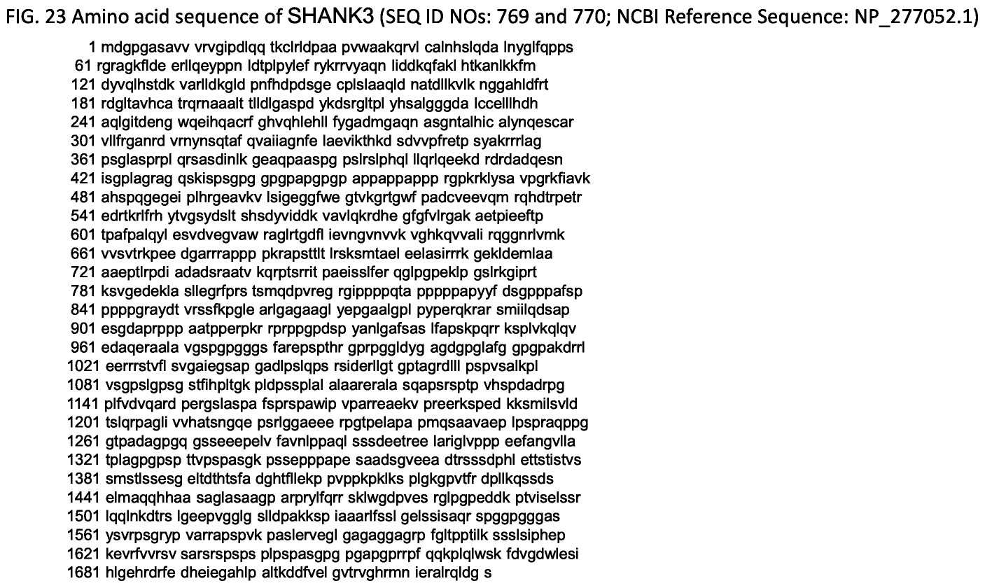

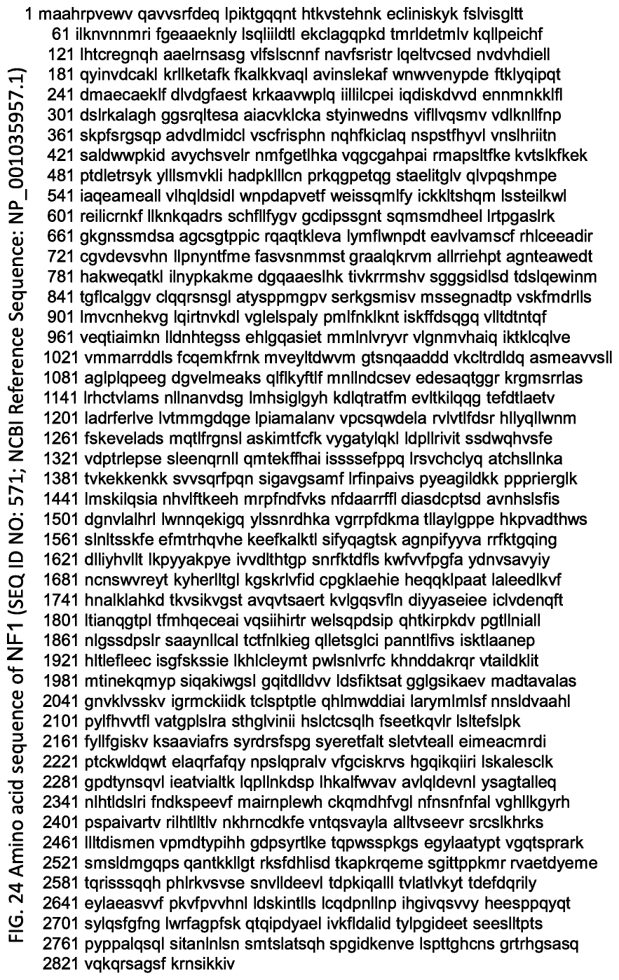

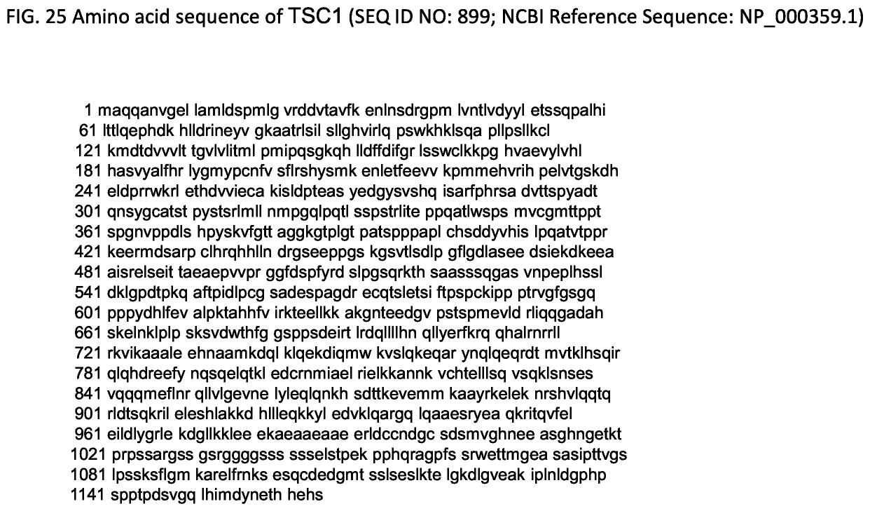

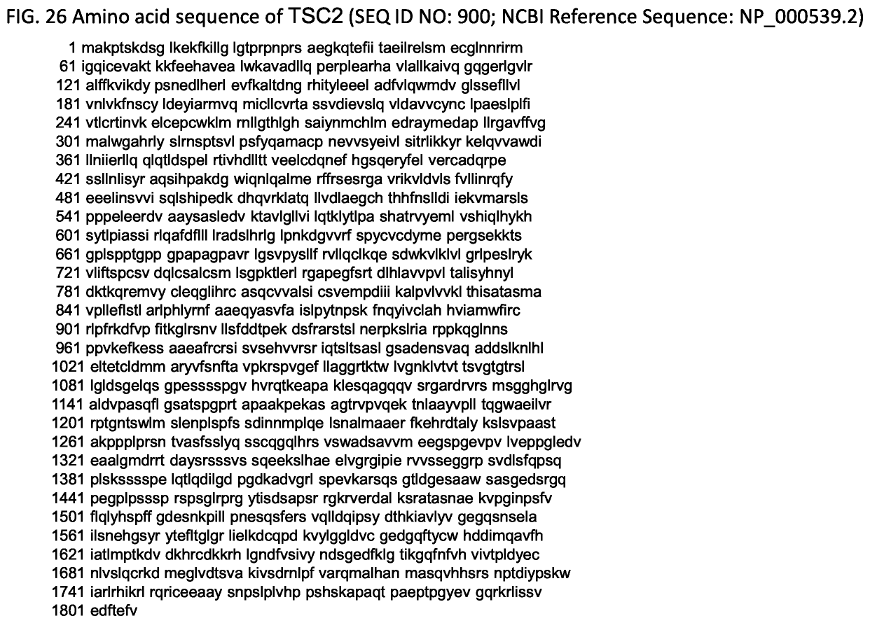

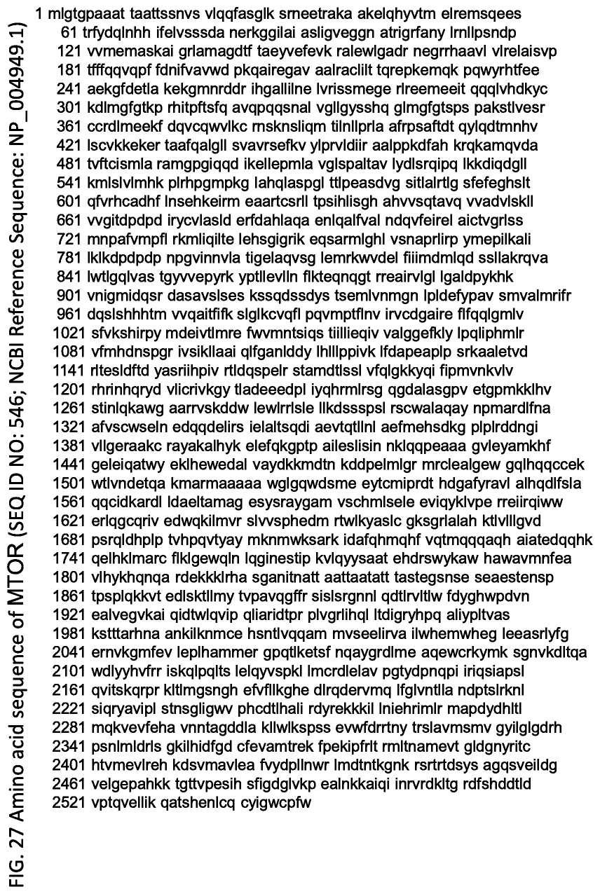

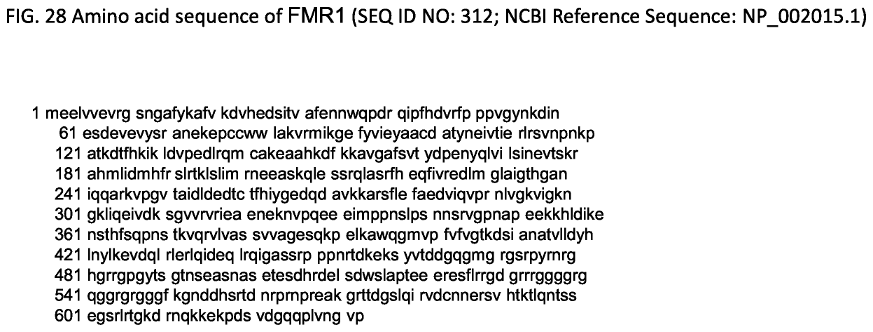

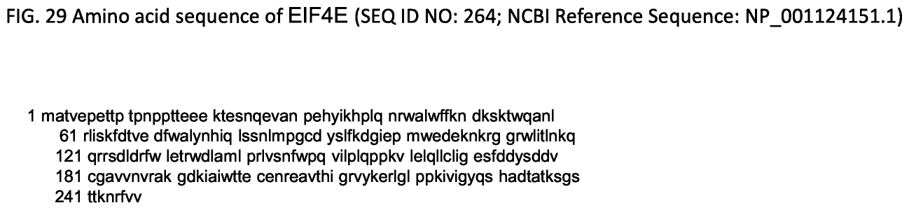

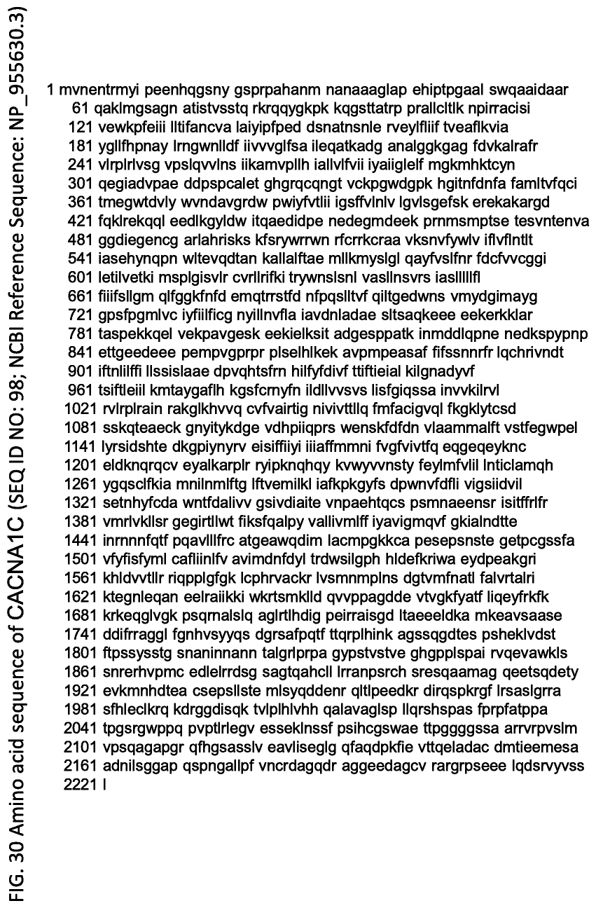

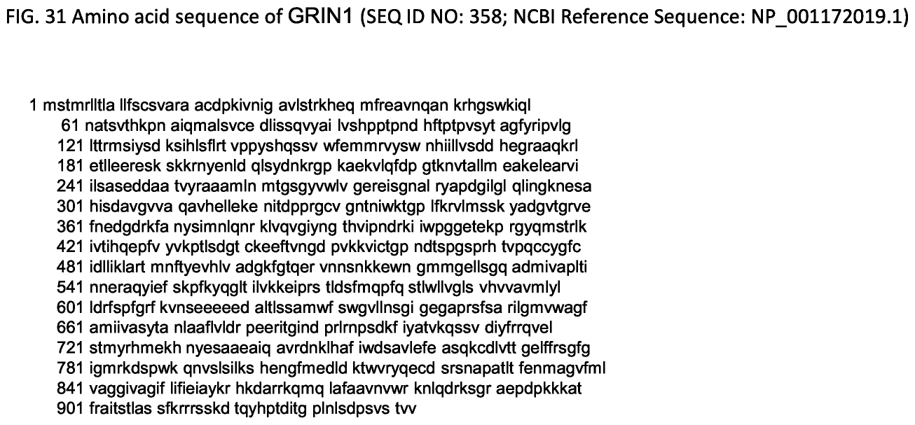

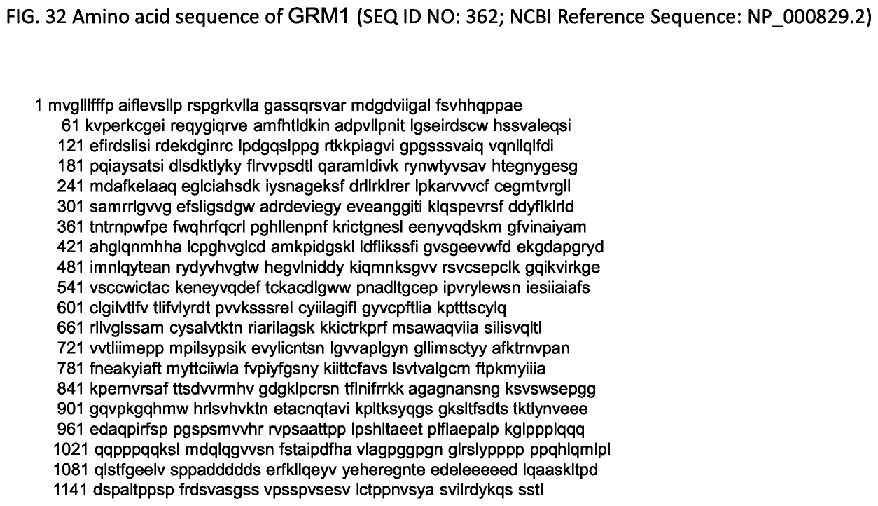

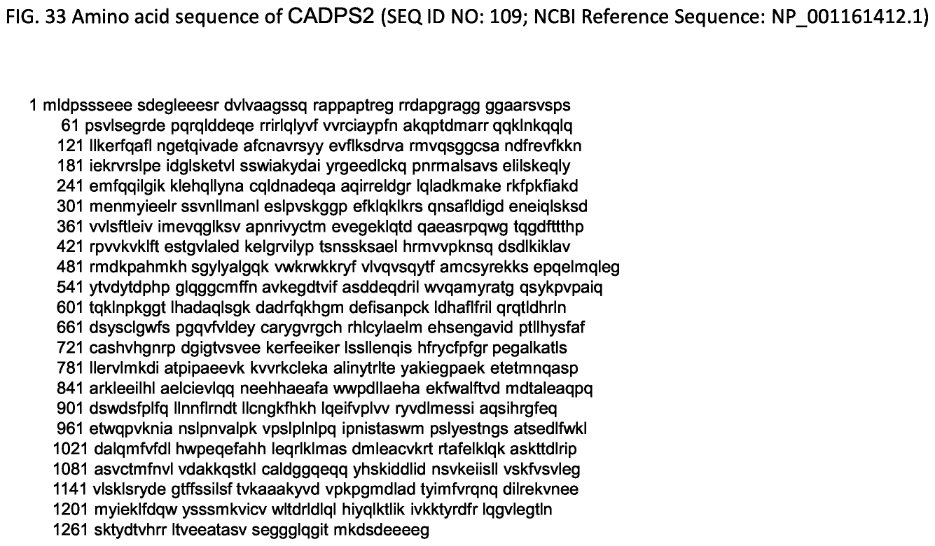

A graphic illustration showing how one mutation in an hnRNP L binding site can affect hnRNP L binding, leading in turn to aberrant processing of the target, is provided in FIGS. 15A and 15B.

HnRNP L as a Modifier of Autism and Candidate RNA targets of hnRNP L

Bioinformatics studies were carried out on human genes linked to ASD.

Table 1 contains a list of 881 human genes linked to autism spectrum disorders. Table 2 contains 338 genes from Table 1 whose genomic sequences (in human genome version GRCh38) include high-scoring, likely hnRNP L binding sites within 500 bp of a documented site of alternative splicing.

SELEX sequences of hnRNP L binding sites from Hui, et al. EMBO J. 2005 Jun. 1; 24(11):1988-98 were used as input to the program MEME (Bailey and Elkan, Proc Int Conf Intell Syst Mol Biol. 1994;2:28-36) to create a position specific state matrix (PSSM) characterizing putative binding sites.

### Log-Likelihood Scores and Position Specific State Matrix (PSSM)

As used herein, the log-likelihood is the base 2 logarithm of the likelihood. Due to its convenience, the log-likelihood was used in place of the likelihood in maximum likelihood estimation of the parameter given a specific dataset and related techniques. The likelihood provides an indication of how much the data contribute to the probability of the parameter value. More rigorously, the likelihood of a parameter value, given specific data, may be the probability of the data given the parameter value.

A position specific scoring matrix (PSSM) is a matrix comprised of log-likelihood scores that compare the probability of seeing the character b in position u of a motif to the probability of seeing b in position u under a random background model. The program MEME (Bailey and Elkan, Proc Int Conf Intell Syst Mol Biol. 1994; 2:28-36) is one method commonly used to infer PSSMs representing common binding motifs from a set of sequences thought to share them. MEME was run on the hnRNPL binding sequences taken from Hui, et al. EMBO J. 2005 Jun. 1; 24(11):1988-98, and obtained the following PSSM, where the rows represent the nucleotides adenine, cytosine, guanine, and thymine, and the columns represent the eight consecutive nucleotides in a putative hnRNP L binding motif.

| | 1 | 2 | 3 | 4 | 5 | 6 | 7 | 8 |
|---|---|---|---|---|---|---|---|---|
| A | 1.644 | −4.000 | 1.700 | −3.000 | 1.672 | −4.000 | 1.907 | −4.000 |
| C | −1.415 | 1.615 | −4.000 | 1.129 | −4.000 | 1.907 | −4.000 | 1.700 |
| G | −2.415 | −4.000 | −0.678 | −4.000 | −1.000 | −3.000 | −3.000 | −4.000 |
| T | −1.678 | −0.300 | −4.000 | 0.700 | −2.000 | −4.000 | −4.000 | −0.678 |

To determine the log-likelihood score of a new 8-mer sequence s under the model, all one needs to do is add up the corresponding scores of the nucleotides in s in the corresponding position. For example, for the sequence CCAAACAC (SEQ ID NO: 977), the relevant entries are bolded:

| | 1 | 2 | 3 | 4 | 5 | 6 | 7 | 8 |
|---|---|---|---|---|---|---|---|---|
| A | 1.644 | −4.000 | 1.700 | −3.000 | 1.672 | −4.000 | 1.907 | −4.000 |
| C | −1.415 | 1.615 | −4.000 | 1.129 | −4.000 | 1.907 | −4.000 | 1.700 |
| G | −2.415 | −4.000 | −0.678 | −4.000 | −1.000 | −3.000 | −3.000 | −4.000 |
| T | −1.678 | −0.300 | −4.000 | 0.700 | −2.000 | −4.000 | −4.000 | −0.678 |

Summing the values in the bolded squares of the matrix gives a total log-likelihood score of 6.086, meaning that the probability of seeing the observed 8-mer CCAAACAC (SEQ ID NO: 977) is at least 67.9 (or 2^{6.086}) times more likely if it is an example of the binding motif than if it were an example of random sequence where each nucleotide is equally likely to occur.

In another example, if the observed sequence were GATTACAG (SEQ ID NO: 978), the bolded matrix would look like that below:

| | 1 | 2 | 3 | 4 | 5 | 6 | 7 | 8 |
|---|---|---|---|---|---|---|---|---|
| A | 1.644 | −4.000 | 1.700 | −3.000 | 1.672 | −4.000 | 1.907 | −4.000 |
| C | −1.415 | 1.615 | −4.000 | 1.129 | −4.000 | 1.907 | −4.000 | 1.700 |
| G | −2.415 | −4.000 | −0.678 | −4.000 | −1.000 | −3.000 | −3.000 | −4.000 |
| T | −1.678 | −0.300 | −4.000 | 0.700 | −2.000 | −4.000 | −4.000 | −0.678 |

The sum of the bolded values is −4.907. The fact that this is negative indicates that the sequence is more likely to have arisen under the random model than under the motif modeled by the PSSM. Specifically, the ratio of the probability of seeing the sequence GATTACAG (SEQ ID NO: 978) under the binding site model to the probability of seeing it under a uniform random model is 2^{-4.907}, which equals 0.0333. In other words, the sequence would be about 30 (approximately 1/0.0333) times more likely to have arisen under the random model than under the PSSM model.

Another example is the sequence ACACACAC (SEQ ID NO:968). This sequence is shown in the matrix below:

| | 1 | 2 | 3 | 4 | 5 | 6 | 7 | 8 |
|---|---|---|---|---|---|---|---|---|
| A | 1.644 | −4.000 | 1.700 | −3.000 | 1.672 | −4.000 | 1.907 | −4.000 |
| C | −1.415 | 1.615 | −4.000 | 1.129 | −4.000 | 1.907 | −4.000 | 1.700 |
| G | −2.415 | −4.000 | −0.678 | −4.000 | −1.000 | −3.000 | −3.000 | −4.000 |
| T | −1.678 | −0.300 | −4.000 | 0.700 | −2.000 | −4.000 | −4.000 | −0.678 |

In this case, the total score is 13.274, the highest that can be found using this matrix. Thus, the sequence is 2^{13.274} (or more than 9905) times more likely to have arisen if the sequence is an hnRNP L binding site than if it came from random sequence.

A sequence logo representing the binding motif thus discovered appears below. This logo differs slightly from that in the Hui, et al. EMBO J. 2005 Jun. 1; 24(11):1988-98 paper, and is shown below

In some embodiments, the hnRNP L binding motif or binding site, described herein, comprises a consensus amino acid sequence shown in the above sequence plot. For example, such hnRNP L binding motif or binding site may comprise an 8-mer amino acid sequence of ACACACAC (SEQ ID NO: 968), ACATACAC (SEQ ID NO: 969), ATACACAC (SEQ ID NO: 970), ATATACAC (SEQ ID NO: 971), ACGCACAC (SEQ ID NO: 972), ACGTACAC (SEQ ID NO: 973), ATGCACAC (SEQ ID NO: 974), ATGTACAC (SEQ ID NO: 975), or X₁X₂X₃X₄X₅CAX₆ (SEQ ID NO: 976), wherein X₁ is A, C, or T, X₂ is C or T, X₃ is A or G, X₄ is C or T, X₅ is A, G, or T, and X₆ is C or T.

Any 8-character sequence (8-mer) can be given a log-likelihood score comparing the probability that the sequence is an example of an hnRNP L binding site to the probability that the sequence arose simply by chance. These log-scaled scores are summed across all positions of the motif, corresponding to the products of their probabilities. An 8-mer having a log-likelihood score of at least 10 means that, across the 8 positions of the motif, the probability of seeing the observed 8-mer is at least 1024 (or 2¹⁰) times more likely if it is an example of the binding motif than if it were an example of random sequence where each nucleotide is equally likely to occur. Similarly, a score of at least 6 means that the sequence is at least 64 (or 2⁶) times more likely to be an example of the motif than not.

Genomic sequences were obtained for all of the genes on the list in Table 1, by using biomaRt to query the GRCh38 assembly in the Ensembl genome database (see Appendices). Screening was carried out, using Perl scripts, to identify each 8-mer in each of the sequences that had high (>=6) or very-high (>=10) scores according to the PSSM representing hnRNP L binding motifs.

24,426 alternative splicing events identified in at least one of the normal tissues tested in Castle, et al., Nature Genetics 40(12):1416-25, 2008 were obtained (see, for example, Castle, et al., Nature Genetics 40(12):1416-25, 2008). The flanking sequences provided for each splice event were matched to the downloaded sequences to identify the Castle splice events' positions in the genome.

A script was then written that compared the high-scoring motifs in each sequence to the locations of the Castle splice events in those genes.

### Castle Splice Site

A “Castle Splice Site” refers to any of the splicing events identified in Castle, et al., Nature Genetics 40(12):1416-25, 2008.

For example, in the gene CD38, position 15,824,682 on the forward strand of chromosome 4 in the GRCh38.p12 primary assembly, marks the start of the splice event called CD38_CASEX_1, a cassette exon (an exon that may be omitted or included in a given transcript; this is sometimes known as “exon skipping”). The sequence from position 15,824,682-15,825,016 corresponds to the potentially skipped exon sequence. Both of these positions, corresponding to both ends of the cassette exon, are counted as splice sites.

In another example, in the gene ST7, position 117,130,606 on the forward strand of chromosome 7, marks the start of the splice event called ST7_MUTEXEX_1, with mutually exclusive exons. Splice sites derived from this event include the 3′ end of the preceding exon, at 117,130,606; both ends of the first mutually exclusive exon, at 117,131,885 and 117,131,960; both ends of the second mutually exclusive exon, at 117,136,081 and 117,136,235; and the 5′ end of the following exon, at 117,138,435.

In another example, in the gene CACNA1G, position 50,619,008 on the forward strand of chromosome 17 markers the start of the splice event called CACNA1G_CASEX2_1, a “double cassette exon” event, in which two consecutive exons may be included or excluded. Splice sites derived from this event include the 3′ end of the preceding exon, at 50,619,008; the 5′ end of the first cassette exon, at 50,619,683; the 3′ end of the second cassette exon, at 50,621,775; and the 5′ end of the following exon, at 50,623,907.

Table 2 reports all of the SFARI genes that have a high-scoring hnRNP L motif within 500 bp of one of the Castle splice sites.

Table 3 reports all the genes that have a very high scoring putative hnRNP L-binding motif within 500 bp of one of the Castle splice sites. There are 152 of these.

Table 4 further connects the SFARI gene list with hnRNP L binding sites near splice events specifically observed in autism. There are 78 such genes.

Table 5 reports all the genes bearing hnRNP L binding sites within the SHANK-TSC-mTOR-ERK ASD disease module. There are 27 of these.

Goldani et al., Front Psychiatry. 2014 Aug. 12; 5:100.

Table 6 reports all the genes bearing hnRNP L binding sites that also comprise the SHANK-TSC ASD disease module (i, ii). There are 18 of these.
(i) Peça J, Feng G. Epub 2012 Mar. 20. Curr Opin Neurobiol. 2012 October; 22(5):866-72.
(ii) Sadakata et al., Int J Environ Res Public Health. 2013 Nov. 27; 10(12):6335-53.

From Parikshak, et al., Nature 540:423-7, 2016, genomic locations were obtained (with respect to the GRCh37 assembly) of 1,127 alternative splicing events, in 833 genes, with a false discovery rate below 0.5 in cerebral cortex of autism patients compared to normal control cortex samples. All SFARI genes that have a high-scoring hnRNP L binding motif within 500 bp of one of these splice sites that Parikshak, et al. have identified as alternatively spliced in cortex in autism spectrum disorders were then identified.

The RNA binding protein, RBFox1/A2BP1, binds to the hexamer UGCAUG (Lee, et al., Neuron 89(1):113-28, 2016; Auweter, et al. EMBO J. 25(1):163-73, 2006). The genomic sequences of the genes in Tables 2-4 were screened for TGCATG hexamers within 200 bp of any of the qualifying hnRNP L motifs that are near the splice sites.

Mutations in the conserved splicing factor FOX1 are linked to autism spectrum disorder (Davis L. K. et al. Am J Med Genet A. 2012;158A:1654-1661; Martin C. L. et al. Am J Med Genet B Neuropsychiatr Genet. 2007;144B:869-876 ;Voineagu I. et al. Nature. 2011;474:380-384; Zhang C. et al. Genes Dev. 2008;22:2550-2563). FIG. 1 is a schematic diagram summarizing the bioinformatic approach.

### Identification of Compounds to Treat Autism Spectrum Disorder

Compounds that modify hnRNP L levels are useful for the development of ASD/mental retardation/neurological disorders. Using Western Blot analysis, it has been observed that ascochlorin treatment of cultured rat primary cortical neurons results in increased levels of hnRNP L.

Cell-Based Assay for the Identification of Compounds that Affect hnRNP L Levels

A cell-based assay using cells and cell lines described herein is used for the identification of additional compounds that affect hnRNP L levels has been developed.

An exemplary method of screening for compounds that repair abnormally spliced genes resulting in or contributing to the severity of neurological disease, autism spectrum disorder, autism, a subset of autism patients (e.g. neurofibromatosis, tuberous sclerosis) is carried out as follows.

### Assay Steps:

1. Identify or generate (e.g. CRISPR, introduction of a synthetic gene construct) a cell line with a specifically abnormally spliced gene.
2. Identify or generate (e.g. CRISPR) a neuronal cell line (e.g. see table) with a specifically abnormally spliced gene.
3. Using established methods, generate iPS derived neurons from relevant patient sample, e.g. biopsy (e.g. skin) or stem cells (isolated from blood)
4. Patients referred to above include those with:
   Neurological Disease
      Spliceopathy induced neurological disease (diagnosed by whole genome sequencing, RNA seq, PCR, etc)
      Autism spectrum disorder
      Autism associated with or caused by a specific splicing event
         e.g., Tuberous sclerosis
         e.g., Neurofibromatosis
         e.g,. subset of patient with spliceopathy of an autism associated gene
5. Plate cells in a multiwell plate (e.g. 96, 384, 1536)
6. Incubate cells (e.g. in triplicate) in the presence and absence of a fixed concentration of compound (e.g. 10-4M, 10-5M, 10-6M) for a specified period of time (e.g. 6 hrs, 12 hrs, 24 hrs, 3 days, 6 days).
7. Compounds include those approved for human use (e.g. FDA approved, European equivalent, etc), compounds established to be safe in man, safe in animal models, analogs of the above, collections of small molecules (e.g. MW<1000, MW<500).
8. Assess the splicing pattern of one or more genes (e.g. Prepare RNA and cDNA corresponding to each well, using transcriptome analysis, multiplex PCR or PCR).
9. With positive compounds:
   Repeat and assess concentration dependent response. Assess analog and derivative activity.
      An exemplary method of screening for compounds that elevate the level of a targeted RNA binding protein acting alone or in combination to modulate splicing (e.g., hnRNPL alone or in complex with other RNA binding proteins to repair abnormally spliced genes contributing to neurological disease) is carried out as follows.

### Assay Steps:

1. Plate cells in a multiwell plate (e.g. 96, 384, 1536) which express mRNA or protein corresponding to the relevant RNA binding protein (e.g. hnRNPL)
   Heterologous cell line
   Neuronal cell line
   Fibroblast derived cell line (from control or affected subject)
   Fibroblast derived neuron (from control or affected subject)
   iPS derived cell line (from control or affected subject)
   iPS derived neuron (from control or affected subject)
   primary neuron (human, rat, mouse, etc)
2. Incubate cells (e.g. in triplicate) in the presence and absence of a fixed concentration of compound (e.g. 10-4M, 10-5M, 10-6M) for a specified period of time (e.g. 6 hrs, 12 hrs, 24 hrs, 3 days, 6 days).
3. Compounds include those approved for human use (e.g. FDA approved, European equivalent, etc), compounds established to be safe in man, safe in animals, analogs of the above, collections of small molecules (e.g. MW<1000, MW<500).
4. Measure the increase in the corresponding mRNA or protein using established methods
   (e.g., PCR, ELISA, luciferase reporter gene activity.
5. Compounds that significantly increase mRNA or protein will be assessed for effects on splicing as detailed above.

Ascochlorin and/or its derivatives can promote the maintenance of normal brain physiology by targeting hnRNP L and/or components of the coordinated hnRNP L-regulated pathway(s). The compounds and methods described herein provide pharmacological leads to help treat autism spectrum disorder and additional neurological and psychiatric disorders.

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated protein or polypeptide is free of the amino acid sequences that flank it in its naturally-occuring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

The term “subject” as used herein includes a patient with a neurological disease. More particularly, the “subject” may include a patient with autism. Autism was first described by Leo Kanner in 1943 and simultaneously by Asperger. Since then, the core symptoms have remained stable. The diagnostic criteria for Autism Spectrum disorder, based on DSM-5 are summarized below:
Social Communication and Social Interaction include:
   Deficits in social emotional reciprocity;
   Deficits in non-verbal communication; and
   Deficits in developing, maintaining and understanding relationships.

Must have all 3 symptoms.
Restricted and Repetitive Behavior including:
   Stereotyped movement or speech;
   Insistence on sameness, routines, rituals;
   Restricted, fixated interests; and
   Atypical sensory reactivity

Must have 2 of 4 symptoms

As per the American Psychiatric Association, symptoms must be present from early childhood and significantly impact function.

The genetics underlying autism are complex as indicated below.
Familial incidence (sibling risk ˜8-12%), High risk sib studies 20%
Twin studies—monozygotic (35-95%) vs. dizygotic (0-25%)
Heritability is estimated at 60-90%

The vast majority of ASD patients carry a clinical but not a genetic diagnosis. With the exponential growth in DNA sequencing capabilities and the decreasing cost of sequencing, new genetic information such as that described herein is emerging. For example, certain disease cohorts with a high occurrence of ASD carry a mutation that is the molecular basis of the accompanying disorder as well as ASD. These ASD subgroups include but are not limited to patients with neurofibromatosis and tuberous sclerosis. In each of these cases a significant fraction of the mutations in the target gene results in spliceopathy. In addition, each of these target genes include hnNPL binding sites raising the possibility that ascochlorin or a related compound that modulates hnRNP L expression/levels could abrogate the spliceopathy and in turn ameliorate the resulting disease.

Accordingly, as used herein, the “patient to be treated” may have a neurological disease. In some embodiments, the “patient” may have a clinical diagnosis of ASD. In some embodiments, the “patient” may have a mutation in a putative ASD target gene which includes hnRNPL binding sites (see Tables 2, 3 and 4). In some embodiments, the “patient” may have a mutation in the target gene which results in spliceopathy. In some embodiments, the “patient” may include known cohorts with neurofibromatosis and tuberous sclerosis who carry a mutation resulting in spliceopathy of the target gene and carry a clinical diagnosis of ASD. Based on ongoing sequencing of large ASD cohorts, it may be anticipated that there will be an expanding number of ASD patient subgroups who fulfill the criteria listed above and are thus candidates for a therapeutic response to ascochlorin and derivatives.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

The term “neurological disorder or disease” as used herein refers to a disorder, disease or condition which directly or indirectly affects the normal functioning or anatomy of a subject's nervous system, including, but not limited to, the brain. In one embodiment, the neurological disorder or disease is a neurodevelopmental disorder.

An example of a neurological disorder or disease is autism. Another example of a neurological disorder or disease is autism spectrum disorder. In other examples, the neurological disorder or disease is epilepsy, schizophrenia or mental retardation.

Autism spectrum disorder (ASD) is a range of complex neurodevelopment disorders, characterized by social impairments, communication difficulties, and restricted, repetitive, and stereotyped patterns of behavior. Autism (also known as autistic disorder or classical ASD) is the most severe form of ASD. Other conditions along the spectrum include Asperger syndrome, childhood disintegrative disorder and pervasive developmental disorder not otherwise specified (also referred to as PDD-NOS), and Chromosome 15q11.2-13.1 duplication syndrome (dup15q syndrome).

The phrase “treating a neurological disorder or disease” as used herein includes, but is not limited to, reversing, alleviating or inhibiting the progression of a neurological disorder or disease or conditions associated with a neurological disorder or disease. As used herein, and as well understood in the art, “to treat” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

Treating a neurological disorder or disease includes preventing the occurrence of a neurological disorder or disease or symptoms or conditions associated with a neurological disorder or disease or preventing worsening of the severity of a neurological disorder or disease or conditions associated with a neurological disorder or disease.

The term “neurological function” as used herein refers to the functioning and/or activity of a subject's nervous system.

The term “improving neurological function” as used herein refers to improving the structure, function and/or activity of a subject's nervous system. In one embodiment, improving neurological function includes improving neurodevelopment and/or improving behavior.

The term “subject” as used herein refers to any member of the animal kingdom, such as a mammal. In one embodiment, the subject is a human. In another embodiment, the subject is a rodent, e.g., mouse or rat, or another animal such as animal model for ASD or intellectual disability.

The term “a cell” includes a single cell as well as a plurality or population of cells. Administering a modulator or an agent to a cell includes both in vitro and in vivo administrations.

The modulators and agents described herein may be formulated into pharmaceutical compositions for administration to subjects and/or use in subjects in a biologically compatible form suitable for administration in vivo. The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Modulators and agents described herein are formulated into pharmaceutical compositions for administration to the brain or central nervous system of a subject. Modulators, agents and pharmaceutical compositions which cannot penetrate the blood-brain barrier can be effectively administered by an intraventricular route or other appropriate delivery system suitable for administration to the brain.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. Proteins may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The modulators, agents and/or pharmaceutical compositions described herein may be administered to, or used in, living organisms including humans, and animals. The term “subject” or “animal” as used herein refers to any member of the animal kingdom, in one embodiment a mammal such as a human being.

Administration of an “effective amount” of the modulators, agents and/or pharmaceutical compositions is defined as an amount effective, at dosages and for periods of time necessary to achieve the desired result. For example, an effective amount of a substance may vary according to factors such as the disease state, age, sex, and weight of the individual, and the ability of the recombinant protein to elicit a desired response in the individual. Dosage regime may be adjusted to provide the optimum therapeutic response. For example, several divided doses may be administered daily or the dose may be proportionally reduced as indicated by the exigencies of the therapeutic situation.

### Compounds for Treatment of ASD and Neurologic Disorders

The potential of ascochlorin (which increases hnRNP L levels ˜12x (Kang J. H. et al. J Proteome Res. 2006;5:2620-2631)) and its derivatives as for the treatment of autism spectrum disorder is underscored by the observation that hnRNP L directly interacts with FOX1. Pharmacological stabilization of the hnRNP L-FOX1 complex may be beneficial in cases where a decrease in the levels of FOX1 (˜5.9 x) is known to cause autism (Voineagu I. et al. Nature. 2011;474:380-384). Further highlighting the potential of ascochlorin and its derivatives for the treatment neurological disorders is evidence that some antibiotics have ancillary neuroprotective effects (Stock M. L. et al. Neuropharmacology. 2013;73C:174-182).

TABLE
Compounds of Formula 1
Formula 1

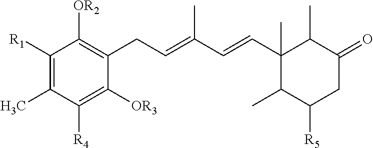

| Com- | | | | | |
|---|---|---|---|---|---|
| pound # | R₁ | R₂ | R₃ | R₄ | R₅ |
| 1 | CHO | H | H | Cl | H |
| 2 | CHO | H | H | Cl | OAc |
| 3 | CHO | H | H | Br | H |
| 4 | CHO | H | H | H | H |
| 5 | CHO | H | CH₃CO | Cl | H |
| 6 | CHO | H | CH₃ | Cl | H |
| 7 | CHO | CH₃ | CH₃ | Cl | H |
| 8 | CHO | CH₃CO | CH₃ | Cl | H |
| 9 | CHO | CH₃ | CH₃CO | Cl | H |
| 10 | CHO | CH₃ | H | Cl | H |
| 11 | CHO | H | CH₃CH₂ | Cl | H |
| 12 | CHO | H | Allyl | Cl | H |
| 13 | CHO | H | Butyl | Cl | H |
| 14 | CHO | H | CH₂COOH | Cl | H |
| 15 | CHO | H | (CH₂)₂COOH | Cl | H |
| 16 | CHO | H | (CH₂)₃COOH | Cl | H |
| 17 | CHO | H | (CH₂)₄COOH | Cl | H |
| 18 | CHO | H | CH₂COOCH₃ | Cl | H |
| 19 | CHO | H | Nicotinoyl | Cl | H |
| 20 | CHO | H | Benzoyl | Cl | H |
| 21 | CHO | H | Isonicotinoyl | Cl | H |
| 22 | CHO | H | CH₂COOC₂H₅ | Cl | H |
| 23 | CHO | H | CH₂COOCH₃ | Cl | H |
| 24 | CHO | H | CH₂COOH | Cl | H |
| 25 | CHO | H | CHCH₃COOC₂H₅ | Cl | H |
| 26 | CHO | H | CHCH₃COOC₄H₉ | Cl | H |
| 27 | CHO | H | CHCH₂CH₃COOC₂H₅ | Cl | H |
| 28 | CHO | H | (CH₂)₃COOC₂H₅ | Cl | H |
| 29 | CHO | H | CHCH₃COOH | Cl | H |
| 30 | CHO | H | (CH₂)₃COOH | Cl | H |
| 31 | CHO | H | Nicotinoyl | Cl | H |
| 32 | CHO | H | COC₆H₄OCH₃ | Cl | H |
| 33 | CHO | H | COC₆H₄COOCH₃ | Cl | H |
| 34 | CHO | H | CON(C₂H₅) | Cl | H |
| 35 | CHO | H | COCH₂OC₆H₄Cl | Cl | H |
| 36 | CHO | H | Isonicotinoyl | Cl | H |
| 37 | CHO | H | Picolinyl | Cl | H |
| 38 | CHO | H | CH₃ | Cl | H |
| 39 | C₂H₂COCH₃ | H | H | Cl | O |
| 40 | CHO | H | CH₃CO | H | H |
| 41 | CHO | H | CH₃ | H | H |
| 42 | C₂H₂COCH₃ | H | CH₂COOH | Cl | O |
| 43 | CHO | CH₃CO | CH₃CO | Cl | H |
| 44 | C(OCH₃)₂ | H | CH₃CO | Cl | H |
| 45 | C(OCH₂CH₃)₂ | H | CH₃CO | Cl | H |
| 46 | C(OCH₂CH₃)₂ | H | CH₃ | Cl | H |
| 47 | C(O(CH₂)₃CH₃)₂ | H | CH₃ | Cl | H |
| 48 | 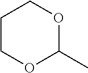 | H | CH₃ | Cl | H |
| 49 | CHO | H | (CH₂)₃CH₃ | Cl | H |
| 50 | CHO | H | CH₂CH₃ | Cl | H |
| 51 | CHO | H | CH₂CHCH₂ | Cl | H |
| 52 | CO₂H | H | H | Cl | H |

TABLE 3
Compounds of Formula 2
Formula 2

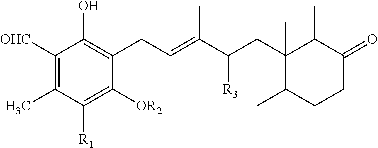

| Compound # | R₁ | R₂ | R₃ |
|---|---|---|---|
| 53 | Cl | H | H |
| 54 | H | H | H |
| 55 | Cl | H | OH |
| 56 | Cl | H | OAc |
| 57 | Cl | CH₃ | H |
| 58 | Cl | CH₃CO | H |
| 59 | H | CH₃ | H |
| 60 | H | CH₃CO | H |
| 61 | Cl | H | OCO(CH₃)₂ |
| 62 | Cl | H | OCOCH₂C(CH₃)₂ |

Additional compounds include, but are not limited to:

3-chloro-4,6-dihydroxy-2-methyl-5-((2E,4E)-3-methyl-5-((1R,2R,6R)-1,2,6-trimethyl-3-oxocyclohexyl)penta-2,4-dien-1-yl)benzaldehyde;

3-chloro-6-hydroxy-4-methoxy-2-methyl-5-((2E,4E)-3-methyl-5-((1R,2R,6R)-1,2,6-trimethyl-3-oxocyclohexyl)penta-2,4-dien-1-yl)benzaldehyde;

2-(2-chloro-4-formyl-5-hydroxy-3-methyl-6-((2E,4E)-3-methyl-5-((1R,2R,6R)-1,2,6-trimethyl-3-oxocyclohexyl)penta-2,4-dien-1-yl)phenoxy)acetic acid;

3-chloro-5-((2E,6E)-7-((S)-5,5-dimethyl-4-oxotetrahydrofuran-2-yl)-3-methylocta-2,6-dien-1-yl)-4,6-dihydroxy-2-methylbenzaldehyde;

(R,E)-5-(3-chloro-5-formyl-2,6-dihydroxy-4-methylphenyl)-3-methyl-1-((1S,2R,6R)-1,2,6-trimethyl-3-oxocyclohexyl)pent-3-en-2-yl butyrate;

3-chloro-4,6-dihydroxy-5-((2E,6E)-7-((2R,3 S)-3-hydroxy-5,5-dimethyl-4-oxotetrahydrofuran-2-yl)-3-methylocta-2,6-dien-1-yl)-2-methylbenzaldehyde;

3-chloro-5-((R,E)-4-(((2R,3R,4R,5S,6R)-3,4-dihydroxy-6-(hydroxymethyl)-5-methoxytetrahydro-2H-pyran-2-yl)oxy)-3-methyl-5-((1S,2R,6R)-1,2,6-trimethyl-3-oxocyclohexyl)pent-2-en-1-yl)-4,6-dihydroxy-2-methylbenzaldehyde;

3-chloro-4,6-dihydroxy-5-((R,E)-4-hydroxy-3-methyl-5-((1S,2R,6R)-1,2,6-trimethyl-3-oxocyclohexyl)pent-2-en-1-yl)-2-methylbenzaldehyde;

3-chloro-4,6-dihydroxy-5-((2E,4E)-5-((1S,2S,3S ,6R)-3-hydroxy-1,2,6-trimethyl-5-oxocyclohexyl)-3-methylpenta-2,4-dien-1-yl)-2-methylbenzaldehyde;

3-chloro-4,6-dihydroxy-2-methyl-5-((2E,4E)-3-methyl-5-((1S ,2R,6R)-1,2,6-trimethyl-5-oxocyclohex-3-en-1-yl)penta-2,4-dien-1-yl)benzaldehyde;

3-chloro-4,6-dihydroxy-5-((2E,4E)-5-((1S,2S,3S ,6R)-3-hydroxy-1,2,6-trimethyl-5-oxocyclohexyl)-3-methylpenta-2,4-dien-1-yl)-2-methylbenzaldehyde;

(E)-3-chloro-5-(3,7-dimethylocta-2,6-dien-1-yl)-4,6-dihydroxy-2-methylbenzaldehyde;

cefacetrile; cefotaxime; ciproflaxin; netilimicine; or a quinolone/fluoroquinolone compound.

TABLE 1
(881 Genes)
SFARI list of autism genes.

| | | SEQ | |
|---|---|---|---|
| Human Gene | Ensembl Identifier | ID NO | Accession Number* |
| ABAT | ENSG00000183044 | 1 | NP_001120920.1 |
| ABCA10 | ENSG00000154263 | 2 | NP_525021.3 |
| ABCA7 | ENSG00000064687 | 3 | NP_061985.2 |
| ACE | ENSG00000159640 | 4 | NP_000780.1 |
| ACHE | ENSG00000087085 | 5 | NP_000656.1 |
| ACTN4 | ENSG00000130402, | 6 | NP_004915.2 |
| | ENSG00000282844 | 7 | |
| ACY1 | ENSG00000243989 | 8 | NP_001185824.1 |
| ADA | ENSG00000196839 | 9 | NP_000013.2 |
| ADAMTS18 | ENSG00000140873 | 10 | NP_955387.1 |
| ADARB1 | ENSG00000197381 | 11 | NP_056648.1 |
| ADCY5 | ENSG00000173175 | 12 | NP_899200.1 |
| ADK | ENSG00000156110 | 13 | NP_001114.2 |
| ADNP | ENSG00000101126 | 14 | NP_056154.1 |
| ADORA2A | ENSG00000128271 | 15 | NP_000666.2 |
| ADORA3 | ENSG00000282608 | 16 | NP_065734.5 |
| ADRB2 | ENSG00000169252 | 17 | NP_000015.1 |
| ADSL | ENSG00000239900 | 18 | NP_000017.1 |
| AFF2 | ENSG00000155966 | 19 | NP_002016.2 |
| AFF4 | ENSG00000072364 | 20 | NP_055238.1 |
| AGAP1 | ENSG00000157985 | 21 | NP_001032208.1 |
| AGBL4 | ENSG00000186094 | 22 | NP_116174.3 |
| AGMO | ENSG00000187546 | 23 | NP_001004320.1 |
| AGTR2 | ENSG00000180772 | 24 | NP_000677.2 |
| AHDC1 | ENSG00000126705 | 25 | NP_001025053.1 |
| AHI1 | ENSG00000135541 | 26 | NP_001128302.1 |
| AKAP9 | ENSG00000127914 | 27 | NP_005742.4 |
| ALDH1A3 | ENSG00000184254 | 28 | NP_000684.2 |
| ALDH5A1 | ENSG00000112294 | 29 | NP_733936.1 |
| ALG6 | ENSG00000088035 | 30 | NP_037471.2 |
| ALOX5AP | ENSG00000132965 | 31 | NP_001191335.1 |
| AMPD1 | ENSG00000116748 | 32 | NP_000027.2 |
| AMT | ENSG00000145020 | 33 | NP_000472.2 |
| ANK2 | ENSG00000145362 | 34 | NP_001139.3 |
| ANK3 | ENSG00000151150 | 35 | NP_066267.2 |
| ANKRD11 | ENSG00000167522 | 36 | NP_001243111.1 |
| ANKS1B | ENSG00000185046 | 37 | NP_690001.3 |
| ANXA1 | ENSG00000135046 | 38 | NP_000691.1 |
| AP1S2 | ENSG00000182287 | 39 | NP_003907.3 |
| APBA2 | ENSG00000034053, | 40 | NP_005494.2 |
| | ENSG00000276495 | 41 | |
| APC | ENSG00000134982 | 42 | NP_001120982.1 |
| APH1A | ENSG00000117362 | 43 | NP_001071096.1 |
| APP | ENSG00000142192 | 44 | NP_000475.1 |
| AR | ENSG00000169083 | 45 | NP_000035.2 |
| ARHGAP11B | ENSG00000187951 | 46 | NP_001034930.1 |
| ARHGAP15 | ENSG00000075884 | 47 | NP_060930.3 |
| ARHGAP24 | ENSG00000138639 | 48 | NP_001020787.2 |
| ARHGAP32 | ENSG00000134909 | 49 | NP_001136157.1 |
| ARHGAP33 | ENSG00000004777 | 50 | NP_443180.2 |
| ARHGEF9 | ENSG00000131089 | 51 | NP_056000.1 |
| ARID1B | ENSG00000049618 | 52 | NP_065783.3 |
| ARNT2 | ENSG00000172379 | 53 | NP_055677.3 |
| ARX | ENSG00000004848 | 54 | NP_620689.1 |
| ASH1L | ENSG00000116539 | 55 | NP_060959.2 |
| ASMT | ENSG00000196433 | 56 | NP_004034.2 |
| ASS1 | ENSG00000130707 | 57 | NP_446464.1 |
| ASTN2 | ENSG00000148219 | 58 | NP_054729.3 |
| ASXL3 | ENSG00000141431 | 59 | NP_085135.1 |
| ATG7 | ENSG00000197548 | 60 | NP_006386.1 |
| ATP10A | ENSG00000206190 | 61 | NP_077816.1 |
| ATP1A3 | ENSG00000105409 | 62 | NP_001243143.1 |
| ATP2B2 | ENSG00000157087 | 63 | NP_001001331.1 |
| ATP8A1 | ENSG00000124406 | 64 | NP_006086.1 |
| ATRNL1 | ENSG00000107518 | 65 | NP_997186.1 |
| ATRX | ENSG00000085224 | 66 | NP_000480.3 |
| ATXN7 | ENSG00000163635 | 67 | NP_001170858.1 |
| AUTS2 | ENSG00000158321 | 68 | NP_056385.1 |
| AVP | ENSG00000101200 | 69 | NP_000481.2 |
| AVPR1A | ENSG00000166148 | 70 | NP_000697.1 |
| AVPR1B | ENSG00000198049 | 71 | NP_000698.1 |
| BAIAP2 | ENSG00000175866 | 72 | NP_059345.1 |
| BBS4 | ENSG00000140463 | 73 | NP_149017.2 |
| BCAS1 | ENSG00000064787 | 74 | NP_003648.2 |
| BCKDK | ENSG00000103507 | 75 | NP_005872.2 |
| BCL11A | ENSG00000119866 | 76 | NP_075044.2 |
| BCL2 | ENSG00000171791 | 77 | NP_000624.2 |
| BDNF | ENSG00000176697 | 78 | NP_001137282.1 |
| BIN1 | ENSG00000136717 | 79 | NP_647593.1 |
| BIRC6 | ENSG00000115760 | 80 | NP_057336.3 |
| BRAF | ENSG00000157764 | 81 | NP_004324.2 |
| BRCA2 | ENSG00000139618 | 82 | NP_000050.2 |
| BRINP1 | ENSG00000078725 | 83 | NP_055433.2 |
| BTAF1 | ENSG00000095564 | 84 | NP_003963.1 |
| BZRAP1 | ENSG00000005379 | 85 | NP_004749.2 |
| C11orf30 | ENSG00000158636 | 86 | NP_064578.2 |
| C12orf57 | ENSG00000111678 | 87 | NP_612434.1 |
| C15orf43 | ENSG00000167014 | 88 | NP_689661.1 |
| C3orf58 | ENSG00000181744 | 89 | NP_775823.1 |
| C4B | ENSG00000236625, | 90 | NP_001229752.1 |
| | ENSG00000224639, | 91 | |
| | ENSG00000228267, | 92 | |
| | ENSG00000228454, | 93 | |
| | ENSG00000224389 | 94 | |
| CA6 | ENSG00000131686 | 95 | NP_001257429.1 |
| CACNA1A | ENSG00000141837 | 96 | NP_075461.2 |
| CACNA1B | ENSG00000148408 | 97 | NP_000709.1 |
| CACNA1C | ENSG00000151067 | 98 | NP_955630.3 |
| CACNA1D | ENSG00000157388 | 99 | NP_000711.1 |
| CACNA1E | ENSG00000198216 | 100 | NP_001192222.1 |
| CACNA1F | ENSG00000102001 | 101 | NP_005174.2 |
| CACNA1G | ENSG00000006283 | 102 | NP_061496.2 |
| CACNA1H | ENSG00000196557 | 103 | NP_066921.2 |
| CACNA1I | ENSG00000100346 | 104 | NP_066919.2 |
| CACNA2D3 | ENSG00000157445 | 105 | NP_060868.2 |
| CACNB2 | ENSG00000165995 | 106 | NP_963 890.2 |
| CADM1 | ENSG00000182985 | 107 | NP_055148.3 |
| CADM2 | ENSG00000175161 | 108 | NP_694854.2 |
| CADPS2 | ENSG00000081803 | 109 | NP_001161412.1 |
| CAMK2A | ENSG00000070808 | 110 | NP_057065.2 |
| CAMK4 | ENSG00000152495 | 111 | NP_001735.1 |
| CAMSAP2 | ENSG00000118200 | 112 | NP_982284.1 |
| CAMTA1 | ENSG00000171735 | 113 | NP_056030.1 |
| CAPN12 | ENSG00000182472 | 114 | NP_653292.2 |
| CAPRIN1 | ENSG00000135387 | 115 | NP_005889.3 |
| CASC4 | ENSG00000166734 | 116 | NP_612432.2 |
| CASK | ENSG00000147044 | 117 | NP_003679.2 |
| CBS | ENSG00000160200 | 118 | NP_001171479.1 |
| CC2D1A | ENSG00000132024 | 119 | NP_060191.3 |
| CCDC64 | ENSG00000135127 | 120 | NP_997194.2 |
| CCDC88C | ENSG00000015133 | 121 | NP_001073883.2 |
| CCDC91 | ENSG00000123106 | 122 | NP_060788.3 |
| CCT4 | ENSG00000115484 | 123 | NP_006421.2 |
| CD38 | ENSG00000004468 | 124 | NP_001766.2 |
| CD44 | ENSG00000026508 | 125 | NP_000601.3 |
| CD99L2 | ENSG00000102181 | 126 | NP_001229543.1 |
| CDC42BPB | ENSG00000198752 | 127 | NP_006026.3 |
| CDH10 | ENSG00000040731 | 128 | NP_006718.2 |
| CDH11 | ENSG00000140937 | 129 | NP_001788.2 |
| CDH22 | ENSG00000149654 | 130 | NP_067071.1 |
| CDH8 | ENSG00000150394 | 131 | NP_001787.2 |
| CDH9 | ENSG00000113100 | 132 | NP_057363.3 |
| CDKL5 | ENSG00000008086 | 133 | NP_003150.1 |
| CDKN1B | ENSG00000111276 | 134 | NP_004055.1 |
| CECR2 | ENSG00000099954 | 135 | NP_113601.2 |
| CELF4 | ENSG00000101489 | 136 | NP_064565.1 |
| CELF6 | ENSG00000140488 | 137 | NP_443072.3 |
| CEP290 | ENSG00000198707 | 138 | NP_079390.3 |
| CEP41 | ENSG00000106477 | 139 | NP_061188.1 |
| CGNL1 | ENSG00000128849 | 140 | NP_116255.2 |
| CHD2 | ENSG00000173575 | 141 | NP_001262.3 |
| CHD7 | ENSG00000171316 | 142 | NP_060250.2 |
| CHD8 | ENSG00000100888 | 143 | NP_001164100.1 |
| CHKB | ENSG00000100288 | 144 | NP_005189.2 |
| CHRM3 | ENSG00000133019 | 145 | NP_000731.1 |
| CHRNA7 | ENSG00000175344 | 146 | NP_001177384.1 |
| CHRNB3 | ENSG00000147432 | 147 | NP_000740.1 |
| CHST5 | ENSG00000135702 | 148 | NP_078809.2 |
| CIB2 | ENSG00000136425 | 149 | NP_006374.1 |
| CLN8 | ENSG00000182372, | 150 | NP_061764.2 |
| | ENSG00000278220 | 151 | |
| CLSTN3 | ENSG00000139182 | 152 | NP_055533.2 |
| CLTCL1 | ENSG00000070371 | 153 | NP_009029.3 |
| CMIP | ENSG00000153815 | 154 | NP_938204.2 |
| CNKSR2 | ENSG00000149970 | 155 | NP_055742.2 |
| CNOT3 | ENSG00000277600, | 156 | NP_055331.1 |
| | ENSG00000274616, | 157 | |
| | ENSG00000277114, | 158 | |
| | ENSG00000088038, | 159 | |
| | ENSG00000273943, | 160 | |
| | ENSG00000274941, | 161 | |
| | ENSG00000274176, | 162 | |
| | ENSG00000277615, | 163 | |
| | ENSG00000276082, | 164 | |
| | ENSG00000275979 | 165 | |
| CNR1 | ENSG00000118432 | 166 | NP_001153698.1 |
| CNR2 | ENSG00000188822 | 167 | NP_001832.1 |
| CNTN3 | ENSG00000113805 | 168 | NP_065923.1 |
| CNTN4 | ENSG00000144619 | 169 | NP_783200.1 |
| CNTN5 | ENSG00000149972 | 170 | NP_001230199.1 |
| CNTN6 | ENSG00000134115 | 171 | NP_055276.1 |
| CNTNAP2 | ENSG00000278728, | 172 | NP_054860.1 |
| | ENSG00000174469 | 173 | |
| CNTNAP3 | ENSG00000106714 | 174 | NP_387504.2 |
| CNTNAP4 | ENSG00000152910 | 175 | NP_207837.2 |
| CNTNAP5 | ENSG00000155052 | 176 | NP_570129.1 |
| CREBBP | ENSG00000005339 | 177 | NP_004371.2 |
| CRHR2 | ENSG00000106113 | 178 | NP_001189404.1 |
| CSMD1 | ENSG00000183117 | 179 | NP_150094.5 |
| CSNK1D | ENSG00000141551 | 180 | NP_001884.2 |
| CTCF | ENSG00000102974 | 181 | NP_006556.1 |
| CTNNA3 | ENSG00000183230 | 182 | NP_037398.2 |
| CTNNB1 | ENSG00000168036 | 183 | NP_001091680.1 |
| CTNND2 | ENSG00000169862 | 184 | NP_001323.1 |
| CTTNBP2 | ENSG00000077063 | 185 | NP_219499.1 |
| CUL3 | ENSG00000036257 | 186 | NP_001244127.1 |
| CUL7 | ENSG00000044090 | 187 | NP_001161842.1 |
| CUX1 | ENSG00000257923 | 188 | NP_001189472.1 |
| CX3CR1 | ENSG00000168329 | 189 | NP_001164645.1 |
| CXCR3 | ENSG00000186810 | 190 | NP_001136269.1 |
| CYFIP1 | ENSG00000273749, | 191 | NP_055423.1 |
| | ENSG00000280618 | 192 | |
| CYLC2 | ENSG00000155833 | 193 | NP_001331.1 |
| CYP11B1 | ENSG00000160882 | 194 | NP_000488.3 |
| DAB1 | ENSG00000173406 | 195 | NP_066566.3 |
| DAGLA | ENSG00000134780 | 196 | NP_006124.1 |
| DAPK1 | ENSG00000196730 | 197 | NP_004929.2 |
| DAPP1 | ENSG00000070190 | 198 | NP_055210.2 |
| DCTN5 | ENSG00000166847 | 199 | NP_115875.1 |
| DCUN1D1 | ENSG00000043093 | 200 | NP_065691.2 |
| DCX | ENSG00000077279 | 201 | NP_000546.2 |
| DDC | ENSG00000132437 | 202 | NP_001076440.1 |
| DDX11 | ENSG00000013573 | 203 | NP_689651.1 |
| DDX3X | ENSG00000215301 | 204 | NP_001347.3 |
| DDX53 | ENSG00000184735 | 205 | NP_874358.2 |
| DEAF1 | ENSG00000177030, | 206 | NP_066288.2 |
| | ENSG00000282712 | 207 | |
| DENR | ENSG00000139726 | 208 | NP_003668.2 |
| DEPDC5 | ENSG00000100150 | 209 | NP_001229825.1 |
| DGKK | ENSG00000274588 | 210 | NP_001013764.1 |
| DGKZ | ENSG00000149091 | 211 | NP_001099010.1 |
| DHCR7 | ENSG00000172893 | 212 | NP_001351.2 |
| DIAPH3 | ENSG00000139734 | 213 | NP_001035982.1 |
| DIP2A | ENSG00000160305 | 214 | NP_055966.2 |
| DISC1 | ENSG00000162946 | 215 | NP_001158009.1 |
| DIXDC1 | ENSG00000150764 | 216 | NP_001033043.1 |
| DLG1 | ENSG00000075711 | 217 | NP_004078.2 |
| DLG4 | ENSG00000132535 | 218 | NP_001356.1 |
| DLGAP1 | ENSG00000170579 | 219 | NP_001229690.1 |
| DLGAP2 | ENSG00000198010, | 220 | NP_004736.2 |
| | ENSG00000282152, | 221 | |
| | ENSG00000274161, | 222 | |
| | ENSG00000282318 | 223 | |
| DLGAP3 | ENSG00000116544 | 224 | NP_001073887.1 |
| DLX1 | ENSG00000144355 | 225 | NP_835221.2 |
| DLX2 | ENSG00000115844 | 226 | NP_004396.1 |
| DLX6 | ENSG00000006377 | 227 | NP_005213.3 |
| DMD | ENSG00000198947 | 228 | NP_003997.1 |
| DMPK | ENSG00000104936 | 229 | NP_001075032.1 |
| DMXL2 | ENSG00000104093 | 230 | NP_001167587.1 |
| DNAH10 | ENSG00000197653, | 231 | NP_997320.2 |
| | ENSG00000281935 | 232 | |
| DNAJC19 | ENSG00000205981 | 233 | NP_660304.1 |
| DNER | ENSG00000187957 | 234 | NP_620711.3 |
| DNM1L | ENSG00000087470 | 235 | NP_036192.2 |
| DNMT3A | ENSG00000119772 | 236 | NP_072046.2 |
| DOCK1 | ENSG00000150760 | 237 | NP_001371.1 |
| DOCK10 | ENSG00000135905 | 238 | NP_055504.2 |
| DOCK4 | ENSG00000128512 | 239 | NP_055520.3 |
| DOCK8 | ENSG00000107099 | 240 | NP_982272.2 |
| DOLK | ENSG00000175283 | 241 | NP_055723.1 |
| DPP10 | ENSG00000175497 | 242 | NP_001171505.1 |
| DPP4 | ENSG00000197635 | 243 | NP_001926.2 |
| DPP6 | ENSG00000130226, | 244 | NP_570629.2 |
| | ENSG00000282974 | 245 | |
| DPYD | ENSG00000188641 | 246 | NP_000101.2 |
| DRD1 | ENSG00000184845 | 247 | NP_000785.1 |
| DRD2 | ENSG00000149295 | 248 | NP_000786.1 |
| DRD3 | ENSG00000151577 | 249 | NP_000787.2 |
| DSCAM | ENSG00000171587 | 250 | NP_001380.2 |
| DST | ENSG00000151914 | 251 | NP_056363.2 |
| DUSP15 | ENSG00000149599 | 252 | NP_542178.2 |
| DUSP22 | ENSG00000112679 | 253 | NP_064570.1 |
| DVL1 | ENSG00000107404 | 254 | NP_004412.2 |
| DVL3 | ENSG00000161202 | 255 | NP_004414.3 |
| DYDC1 | ENSG00000170788 | 256 | NP_620167.1 |
| DYDC2 | ENSG00000133665 | 257 | NP_001256971.1 |
| DYRK1A | ENSG00000157540 | 258 | NP_001387.2 |
| EEF1A2 | ENSG00000101210 | 259 | NP_001949.1 |
| EFR3A | ENSG00000132294 | 260 | NP_055952.2 |
| EGR2 | ENSG00000122877 | 261 | NP_001129649.1 |
| EHMT1 | ENSG00000181090 | 262 | NP_079033.4 |
| EIF3G | ENSG00000130811 | 263 | NP_003746.2 |
| EIF4E | ENSG00000151247 | 264 | NP_001124151.1 |
| EIF4EBP2 | ENSG00000148730 | 265 | NP_004087.1 |
| ELAVL2 | ENSG00000107105 | 266 | NP_004423.2 |
| ELAVL3 | ENSG00000196361 | 267 | NP_001411.2 |
| ELP4 | ENSG00000109911 | 268 | NP_061913.3 |
| EML1 | ENSG00000066629 | 269 | NP_001008707.1 |
| EN2 | ENSG00000164778 | 270 | NP_001418.2 |
| EP300 | ENSG00000100393 | 271 | NP_001420.2 |
| EP400 | ENSG00000183495 | 272 | NP_056224.3 |
| EPC2 | ENSG00000135999 | 273 | NP_056445.3 |
| EPHA6 | ENSG00000080224 | 274 | NP_001073917.2 |
| EPHB2 | ENSG00000133216 | 275 | NP_004433.2 |
| EPHB6 | ENSG00000275482, | 276 | NP_004436.2 |
| | ENSG00000106123 | 277 | |
| EPS8 | ENSG00000151491 | 278 | NP_004438.3 |
| ERBB4 | ENSG00000178568 | 279 | NP_005226.1 |
| ERBIN | ENSG00000112851 | 280 | NP_001240628.1 |
| ERG | ENSG00000157554 | 281 | NP_001129626.1 |
| ERMN | ENSG00000136541 | 282 | NP_001009959.1 |
| ESR1 | ENSG00000091831 | 283 | NP_001116212.1 |
| ESR2 | ENSG00000140009 | 284 | NP_001428.1 |
| ESRRB | ENSG00000119715 | 285 | NP_004443.3 |
| ETFB | ENSG00000105379 | 286 | NP_001014763.1 |
| EXOC3 | ENSG00000180104 | 287 | NP_009208.2 |
| EXOC5 | ENSG00000070367 | 288 | NP_006535.1 |
| EXOC6 | ENSG00000138190 | 289 | NP_061926.3 |
| EXOC6B | ENSG00000144036 | 290 | NP_056004.1 |
| EXT1 | ENSG00000182197 | 291 | NP_000118.2 |
| F13A1 | ENSG00000124491 | 292 | NP_000120.2 |
| FABP3 | ENSG00000121769 | 293 | NP_004093.1 |
| FABP5 | ENSG00000164687 | 294 | NP_001435.1 |
| FABP7 | ENSG00000164434 | 295 | NP_001437.1 |
| FAM135B | ENSG00000147724 | 296 | NP_056996.2 |
| FAM92B | ENSG00000153789 | 297 | NP_940893.1 |
| FAN1 | ENSG00000198690, | 298 | NP_055782.3 |
| | ENSG00000276787 | 299 | |
| FAT1 | ENSG00000083857 | 300 | NP_005236.2 |
| FBN1 | ENSG00000166147 | 301 | NP_000129.3 |
| FBXO15 | ENSG00000141665 | 302 | NP_001136430.1 |
| FBXO33 | ENSG00000165355 | 303 | NP_976046.1 |
| FBXO40 | ENSG00000163833 | 304 | NP_057382.2 |
| FER | ENSG00000151422 | 305 | NP_005237.2 |
| FEZF2 | ENSG00000153266 | 306 | NP_060478.3 |
| FGA | ENSG00000171560 | 307 | NP_000499.1 |
| FGD1 | ENSG00000102302 | 308 | NP_004454.2 |
| FGFBP3 | ENSG00000174721 | 309 | NP_689642.3 |
| FHIT | ENSG00000189283 | 310 | NP_001159715.1 |
| FLT1 | ENSG00000102755 | 311 | NP_002010.2 |
| FMR1 | ENSG00000102081 | 312 | NP_002015.1 |
| FOLH1 | ENSG00000086205 | 313 | NP_004467.1 |
| FOXG1 | ENSG00000176165 | 314 | NP_005240.3 |
| FOXP1 | ENSG00000114861 | 315 | NP_001231739.1 |
| FOXP2 | ENSG00000128573 | 316 | NP_683696.2 |
| FRK | ENSG00000111816 | 317 | NP_002022.1 |
| FRMPD4 | ENSG00000169933 | 318 | NP_055543.2 |
| GABRA1 | ENSG00000022355 | 319 | NP_001121115.1 |
| GABRA3 | ENS G00000011677 | 320 | NP_000799.1 |
| GABRA4 | ENSG00000109158 | 321 | NP_000800.2 |
| GABRA5 | ENSG00000186297 | 322 | NP_001158509.1 |
| GABRB1 | ENSG00000163288 | 323 | NP_000803.2 |
| GABRB3 | ENSG00000166206 | 324 | NP_068712.1 |
| GABRQ | ENSG00000268089 | 325 | NP_061028.2 |
| GAD1 | ENSG00000128683 | 326 | NP_000808.2 |
| GADD45B | ENSG00000099860 | 327 | NP_056490.2 |
| GALNT13 | ENSG00000144278 | 328 | NP_443149.2 |
| GALNT14 | ENSG00000158089 | 329 | NP_001240755.1 |
| GAN | ENSG00000261609 | 330 | NP_071324.1 |
| GAP43 | ENSG00000172020 | 331 | NP_001123536.1 |
| GAS2 | ENSG00000148935 | 332 | NP_001137302.1 |
| GATM | ENSG00000171766 | 333 | NP_001473.1 |
| GDA | ENSG00000119125 | 334 | NP_001229434.1 |
| GIGYF1 | ENSG00000146830 | 335 | NP_072096.2 |
| GIGYF2 | ENSG00000204120 | 336 | NP_001096617.1 |
| GLIS1 | ENSG00000174332 | 337 | NP_671726.2 |
| GLO1 | ENSG00000124767 | 338 | NP_006699.2 |
| GLRA2 | ENSG00000101958 | 339 | NP_001112357.1 |
| GNA14 | ENSG00000156049 | 340 | NP_004288.1 |
| GNAS | ENSG00000087460 | 341 | NP_536350.2 |
| GNB1L | ENSG00000185838 | 342 | NP_443730.1 |
| GPC4 | ENSG00000076716 | 343 | NP_001439.2 |
| GPC6 | ENSG00000183098 | 344 | NP_005699.1 |
| GPD2 | ENSG00000115159 | 345 | NP_001076581.2 |
| GPHN | ENSG00000171723 | 346 | NP_065857.1 |
| GPR139 | ENSG00000180269 | 347 | NP_001002911.1 |
| GPR37 | ENSG00000170775 | 348 | NP_005293.1 |
| GPR85 | ENSG00000164604 | 349 | NP_061843.3 |
| GPX1 | ENSG00000233276 | 350 | NP_000572.2 |
| GRID1 | ENSG00000182771 | 351 | NP_060021.1 |
| GRID2 | ENSG00000152208 | 352 | NP_001501.2 |
| GRID2IP | ENSG00000215045 | 353 | NP_001138590.1 |
| GRIK2 | ENSG00000164418 | 354 | NP_068775.1 |
| GRIK3 | ENSG00000163873 | 355 | NP_000822.2 |
| GRIK4 | ENSG00000149403 | 356 | NP_055434.2 |
| GRIK5 | ENSG00000105737 | 357 | NP_002079.3 |
| GRIN1 | ENSG00000176884 | 358 | NP_001172019.1 |
| GRIN2A | ENSG00000183454 | 359 | NP_001127879.1 |
| GRIN2B | ENSG00000273079 | 360 | NP_000825.2 |
| GRIP1 | ENSG00000155974 | 361 | NP_066973.2 |
| GRM1 | ENSG00000152822 | 362 | NP_000829.2 |
| GRM4 | ENSG00000124493 | 363 | NP_000832.1 |
| GRM5 | ENSG00000168959 | 364 | NP_001137303.1 |
| GRM7 | ENSG00000196277 | 365 | NP_870989.1 |
| GRM8 | ENSG00000179603 | 366 | NP_000836.2 |
| GRPR | ENSG00000126010 | 367 | NP_005305.1 |
| GSK3B | ENSG00000082701 | 368 | NP_002084.2 |
| GSN | ENSG00000148180 | 369 | NP_000168.1 |
| GSTM1 | ENSG00000134184 | 370 | NP_000552.2 |
| GTF2I | ENSG00000263001 | 371 | NP_127492.1 |
| GUCY1A2 | ENSG00000152402 | 372 | NP_001243353.1 |
| HCFC1 | ENSG00000172534 | 373 | NP_005325.2 |
| HCN1 | ENSG00000164588 | 374 | NP_066550.2 |
| HDAC3 | ENSG00000171720 | 375 | NP_003874.2 |
| HDAC4 | ENSG00000068024 | 376 | NP_006028.2 |
| HDAC6 | ENSG00000094631 | 377 | NP_006035.2 |
| HDC | ENSG00000140287 | 378 | NP_002103.2 |
| HECW2 | ENSG00000138411 | 379 | NP_065811.1 |
| HEPACAM | ENSG00000165478 | 380 | NP_689935.2 |
| HERC2 | ENSG00000276802, | 381 | NP_004658.3 |
| | ENSG00000128731, | 382 | |
| | ENSG00000277278 | 383 | |
| HIVEP3 | ENSG00000127124 | 384 | NP_078779.2 |
| HLA-A | ENSG00000235657, | 385 | NP_001229687.1 |
| | ENSG00000223980, | 386 | |
| | ENSG00000206503, | 387 | |
| | ENSG00000206505, | 388 | |
| | ENSG00000227715, | 389 | |
| | ENSG00000231834, | 390 | |
| | ENSG00000224320, | 391 | |
| | ENSG00000229215 | 392 | |
| HLA-B | ENSG00000223532, | 393 | NP_005505.2 |
| | ENSG00000206450, | 394 | |
| | ENSG00000234745, | 395 | |
| | ENSG00000224608, | 396 | |
| | ENSG00000228964, | 397 | |
| | ENSG00000232126 | 398 | |
| HLA-DRB1 | ENSG00000236884, | 399 | NP_001230894.1 |
| | ENSG00000228080, | 400 | |
| | ENSG00000206240, | 401 | |
| | ENSG00000229074, | 402 | |
| | ENSG00000206306, | 403 | |
| | ENSG00000196126 | 404 | |
| HMGN1 | ENSG00000205581 | 405 | NP_004956.5 |
| HNRNPH2 | ENSG00000126945 | 406 | NP_001027565.1 |
| HOMER1 | ENSG00000152413 | 407 | NP_004263.1 |
| HOXA1 | ENSG00000105991 | 408 | NP_005513.1 |
| HOXB1 | ENSG00000120094 | 409 | NP_002135.2 |
| HRAS | ENSG00000174775, | 410 | NP_001123914.1 |
| | ENSG00000276536 | 411 | |
| HS3ST5 | ENSG00000249853 | 412 | NP_705840.2 |
| HSD11B1 | ENSG00000117594 | 413 | NP_861420.1 |
| HTR1B | ENSG00000135312 | 414 | NP_000854.1 |
| HTR2A | ENSG00000102468 | 415 | NP_000612.1 |
| HTR3A | ENSG00000166736 | 416 | NP_998786.2 |
| HTR3C | ENSG00000178084 | 417 | NP_570126.2 |
| HTR7 | ENSG00000148680 | 418 | NP_062873.1 |
| HUWE1 | ENSG00000086758 | 419 | NP_113584.3 |
| HYDIN | ENSG00000157423, | 420 | NP_001257903.1 |
| | ENSG00000283022 | 421 | |
| ICA1 | ENSG00000003147 | 422 | NP_071682.1 |
| IFNG | ENSG00000111537 | 423 | NP_000610.2 |
| IFNGR1 | ENSG00000027697 | 424 | NP_000407.1 |
| IL16 | ENSG00000172349 | 425 | NP_757366.2 |
| IL17A | ENSG00000112115 | 426 | NP_002181.1 |
| IL1R2 | ENSG00000115590 | 427 | NP_004624.1 |
| IL1RAPL1 | ENSG00000169306 | 428 | NP_055086.1 |
| IL1RAPL2 | ENSG00000189108 | 429 | NP_059112.1 |
| IL6 | ENSG00000136244 | 430 | NP_000591.1 |
| ILF2 | ENSG00000143621 | 431 | NP_004506.2 |
| IMMP2L | ENSG00000184903 | 432 | NP_115938.1 |
| INADL | ENSG00000132849 | 433 | NP_795352.2 |
| INPP1 | ENSG00000151689 | 434 | NP_001122400.1 |
| INTS6 | ENSG00000102786 | 435 | NP_036273.1 |
| IQGAP3 | ENSG00000183856 | 436 | NP_839943.2 |
| IQSEC2 | ENSG00000124313 | 437 | NP_001104595.1 |
| IRF2BPL | ENSG00000119669 | 438 | NP_078772.1 |
| ITGA4 | ENSG00000115232 | 439 | NP_000876.3 |
| ITGB3 | ENSG00000259207 | 440 | NP_000203.2 |
| ITGB7 | ENSG00000139626 | 441 | NP_000880.1 |
| ITPR1 | ENSG00000150995 | 442 | NP_001161744.1 |
| JAKMIP1 | ENSG00000152969 | 443 | NP_001092903.1 |
| JARID2 | ENSG00000008083 | 444 | NP_004964.2 |
| JMJD1C | ENSG00000171988 | 445 | NP_116165.1 |
| KANK1 | ENSG00000107104 | 446 | NP_001243805.1 |
| KAT2B | ENSG00000114166 | 447 | NP_003875.3 |
| KAT6A | ENSG00000083168 | 448 | NP_001092882.1 |
| KATNAL2 | ENSG00000167216 | 449 | NP_112593.2 |
| KCND2 | ENSG00000184408 | 450 | NP_036413.1 |
| KCNJ10 | ENSG00000177807 | 451 | NP_002232.2 |
| KCNJ12 | ENSG00000184185 | 452 | NP_066292.2 |
| KCNJ15 | ENSG00000157551 | 453 | NP_002234.2 |
| KCNJ2 | ENSG00000123700 | 454 | NP_000882.1 |
| KCNMA1 | ENSG00000156113 | 455 | NP_001154824.1 |
| KCNQ2 | ENSG00000281151, | 456 | NP_742105.1 |
| | ENSG00000075043 | 457 | |
| KCNQ3 | ENSG00000184156 | 458 | NP_004510.1 |
| KCNT1 | ENSG00000107147 | 459 | NP_065873.2 |
| KCTD13 | ENSG00000174943 | 460 | NP_849194.1 |
| KDM4B | ENSG00000127663 | 461 | NP_055830.1 |
| KDM5B | ENSG00000117139 | 462 | NP_006609.3 |
| KDM5C | ENSG00000126012 | 463 | NP_004178.2 |
| KDM6B | ENSG00000132510 | 464 | NP_001073893.1 |
| KHDRBS2 | ENSG00000112232 | 465 | NP_689901.2 |
| KHDRBS3 | ENSG00000131773 | 466 | NP_006549.1 |
| KIAA1586 | ENSG00000168116 | 467 | NP_065982.1 |
| KIAA2022 | ENSG00000050030 | 468 | NP_001008537.1 |
| KIF13B | ENSG00000197892 | 469 | NP_056069.2 |
| KIF5C | ENSG00000276734, | 470 | NP_004513.1 |
| | ENSG00000168280 | 471 | |
| KIRREL3 | ENSG00000149571 | 472 | NP_115920.1 |
| KIT | ENSG00000157404 | 473 | NP_000213.1 |
| KLC2 | ENSG00000174996 | 474 | NP_073733.1 |
| KMO | ENSG00000117009 | 475 | NP_003670.2 |
| KMT2A | ENSG00000118058 | 476 | NP_005924.2 |
| KMT2C | ENSG00000055609 | 477 | NP_733751.2 |
| KMT2E | ENSG00000005483 | 478 | NP_891847.1 |
| KMT5B | ENSG00000110066 | 479 | NP_060105.3 |
| KPTN | ENSG00000118162 | 480 | NP_008990.2 |
| KRR1 | ENSG00000111615 | 481 | NP_008974.5 |
| LAMA1 | ENSG00000101680 | 482 | NP_005550.2 |
| LAMB1 | ENSG00000091136 | 483 | NP_002282.2 |
| LAMC3 | ENSG00000050555 | 484 | NP_006050.3 |
| LEP | ENSG00000174697 | 485 | NP_000221.1 |
| LIN7B | ENSG00000104863 | 486 | NP_071448.1 |
| LMX1B | ENSG00000136944 | 487 | NP_001167617.1 |
| LPL | ENSG00000175445 | 488 | NP_000228.1 |
| LRBA | ENSG00000198589 | 489 | NP_006717.2 |
| LRFN5 | ENSG00000165379 | 490 | NP_689660.2 |
| LRP2 | ENSG00000081479 | 491 | NP_004516.2 |
| LRP2BP | ENSG00000109771 | 492 | NP_060879.2 |
| LRPPRC | ENSG00000138095 | 493 | NP_573566.2 |
| LRRC1 | ENSG00000137269 | 494 | NP_060684.4 |
| LRRC7 | ENSG00000033122 | 495 | NP_065845.1 |
| LZTR1 | ENSG00000099949 | 496 | NP_006758.2 |
| LZTS2 | ENSG00000107816 | 497 | NP_115805.1 |
| MACROD2 | ENSG00000172264 | 498 | NP_542407.2 |
| MAGED1 | ENSG00000179222 | 499 | NP_001005333.1 |
| MAGEL2 | ENSG00000254585 | 500 | NP_061939.3 |
| MAL | ENSG00000172005 | 501 | NP_002362.1 |
| MAOA | ENSG00000189221 | 502 | NP_000231.1 |
| MAOB | ENSG00000069535 | 503 | NP_000889.3 |
| MAP2 | ENSG00000078018 | 504 | NP_002365.3 |
| MAPK1 | ENSG00000100030 | 505 | NP_620407.1 |
| MAPK12 | ENSG00000188130 | 506 | NP_002960.2 |
| MAPK3 | ENSG00000102882 | 507 | NP_002737.2 |
| MAPK8IP2 | ENSG00000008735 | 508 | NP_036456.1 |
| MARK1 | ENSG00000116141 | 509 | NP_061120.3 |
| MBD1 | ENSG00000141644 | 510 | NP_001191065.1 |
| MBD3 | ENSG00000071655 | 511 | NP_003917.1 |
| MBD4 | ENSG00000129071 | 512 | NP_003916.1 |
| MBD5 | ENSG00000204406 | 513 | NP_060798.2 |
| MBD6 | ENSG00000166987 | 514 | NP_443129.3 |
| MBOAT7 | ENSG00000275118, | 515 | NP_077274.3 |
| | ENSG00000273592, | 516 | |
| | ENSG00000125505, | 517 | |
| | ENSG00000276935, | 518 | |
| | ENSG00000278519, | 519 | |
| | ENSG00000277733, | 520 | |
| | ENSG00000278322, | 521 | |
| | ENSG00000274194, | 522 | |
| | ENSG00000277025, | 523 | |
| | ENSG00000277923 | 524 | |
| MC4R | ENSG00000166603 | 525 | NP_005903.2 |
| MCC | ENSG00000171444 | 526 | NP_001078846.1 |
| MCM4 | ENSG00000104738 | 527 | NP_877423.1 |
| MCPH1 | ENSG00000147316 | 528 | NP_078872.2 |
| MDGA2 | ENSG00000139915 | 529 | NP_001106970.2 |
| MECP2 | ENSG00000169057 | 530 | NP_001104262.1 |
| MED12 | ENSG00000184634 | 531 | NP_005111.2 |
| MED13L | ENSG00000123066 | 532 | NP_056150.1 |
| MEF2C | ENSG00000081189 | 533 | NP_001180276.1 |
| MET | ENSG00000105976 | 534 | NP_001120972.1 |
| MFRP | ENSG00000235718 | 535 | NP_113621.1 |
| MIB1 | ENSG00000101752 | 536 | NP_065825.1 |
| MKL2 | ENSG00000186260 | 537 | NP_054767.3 |
| MNT | ENSG00000070444 | 538 | NP_064706.1 |
| MOCOS | ENSG00000075643 | 539 | NP_060417.2 |
| MPP6 | ENSG00000105926 | 540 | NP_057531.2 |
| MSN | ENSG00000147065 | 541 | NP_002435.1 |
| MSNP1AS | ENSG00000251593 | 542 | |
| MSR1 | ENSG00000038945 | 543 | NP_619729.1 |
| MTF1 | ENSG00000188786 | 544 | NP_005946.2 |
| MTHFR | ENSG00000177000 | 545 | NP_005948.3 |
| MTOR | ENSG00000198793 | 546 | NP_004949.1 |
| MTR | ENSG00000116984 | 547 | NP_000245.2 |
| MTX2 | ENSG00000128654 | 548 | NP_006545.1 |
| MYH4 | ENSG00000264424 | 549 | NP_060003.2 |
| MYO16 | ENSG00000041515, | 550 | NP_001185879.1 |
| | ENSG00000282848 | 551 | |
| MYO1A | ENSG00000166866 | 552 | NP_001242970.1 |
| MYO9B | ENSG00000099331 | 553 | NP_004136.2 |
| MYOZ1 | ENSG00000177791 | 554 | NP_067068.1 |
| MYT1L | ENSG00000186487 | 555 | NP_055840.2 |
| NAA15 | ENSG00000164134 | 556 | NP_476516.1 |
| NAALADL2 | ENSG00000177694 | 557 | NP_996898.2 |
| NACC1 | ENSG00000160877 | 558 | NP_443108.1 |
| NAV2 | ENSG00000166833 | 559 | NP_001231892.1 |
| NBEA | ENSG00000172915 | 560 | NP_056493.3 |
| NCKAP1 | ENSG00000061676 | 561 | NP_995314.1 |
| NCKAP5 | ENSG00000176771 | 562 | NP_997246.2 |
| NCKAP5L | ENSG00000167566 | 563 | NP_001032895.2 |
| NCOR1 | ENSG00000141027 | 564 | NP_006302.2 |
| NDNL2 | ENSG00000185115, | 565 | NP_619649.1 |
| | ENSG00000282130 | 566 | |
| NDUFA5 | ENSG00000128609 | 567 | NP_004991.1 |
| NEFL | ENSG00000277586 | 568 | NP_006149.2 |
| NELL1 | ENSG00000165973 | 569 | NP_006148.2 |
| NEO1 | ENSG00000067141 | 570 | NP_002490.2 |
| NF1 | ENSG00000196712 | 571 | NP_001035957.1 |
| NFIA | ENSG00000162599 | 572 | NP_001138984.1 |
| NFIX | ENSG00000008441 | 573 | NP_001257972.1 |
| NINL | ENSG00000101004 | 574 | NP_079452.3 |
| NIPA1 | ENSG00000170113 | 575 | NP_653200.2 |
| NIPA2 | ENSG00000140157 | 576 | NP_001171818.1 |
| NIPBL | ENSG00000164190 | 577 | NP_597677.2 |
| NLGN1 | ENSG00000169760 | 578 | NP_055747.1 |
| NLGN2 | ENSG00000169992, | 579 | NP_065846.1 |
| | ENSG00000283859 | 580 | |
| NLGN3 | ENSG00000196338 | 581 | NP_851820.1 |
| NLGN4X | ENSG00000146938 | 582 | NP_065793.1 |
| NLGN4Y | ENSG00000165246 | 583 | NP_055708.3 |
| NOS1 | ENSG00000089250 | 584 | NP_001191147.1 |
| NOS1AP | ENSG00000198929 | 585 | NP_055512.1 |
| NOS2A | ENSG00000007171 | 586 | NP_000616.3 |
| NPAS2 | ENSG00000170485 | 587 | NP_002509.2 |
| NR2F1 | ENSG00000175745 | 588 | NP_005645.1 |
| NR3C2 | ENSG00000151623 | 589 | NP_000892.2 |
| NRCAM | ENSG00000091129 | 590 | NP_001032209.1 |
| NRG1 | ENSG00000157168 | 591 | NP_039250.2 |
| NRP2 | ENSG00000118257 | 592 | NP_957718.1 |
| NRXN1 | ENSG00000179915 | 593 | NP_001129131.1 |
| NRXN2 | ENSG00000110076 | 594 | NP_055895.1 |
| NRXN3 | ENSG00000021645 | 595 | NP_004787.2 |
| NSD1 | ENSG00000165671 | 596 | NP_071900.2 |
| NTNG1 | ENSG00000162631 | 597 | NP_001106697.1 |
| NTRK1 | ENSG00000198400 | 598 | NP_002520.2 |
| NTRK3 | ENSG00000140538 | 599 | NP_001012338.1 |
| NUAK1 | ENSG00000074590 | 600 | NP_055655.1 |
| NUP133 | ENSG00000069248 | 601 | NP_060700.2 |
| NXF5 | ENSG00000126952 | 602 | NP_116564.2 |
| NXPH1 | ENSG00000122584 | 603 | NP_689958.1 |
| OCRL | ENSG00000122126 | 604 | NP_000267.2 |
| ODF3L2 | ENSG00000181781 | 605 | NP_872383.1 |
| OGT | ENSG00000147162 | 606 | NP_858058.1 |
| OPHN1 | ENSG00000079482 | 607 | NP_002538.1 |
| OPRM1 | ENSG00000112038 | 608 | NP_001138751.1 |
| OR1C1 | ENSG00000221888 | 609 | NP_036485.2 |
| OR2M4 | ENSG00000171180 | 610 | NP_059974.1 |
| OR2T10 | ENSG00000184022 | 611 | NP_001004693.1 |
| OR52M1 | ENSG00000197790 | 612 | NP_001004137.1 |
| OTX1 | ENSG00000115507 | 613 | NP_055377.1 |
| OXT | ENSG00000101405 | 614 | NP_000906.1 |
| OXTR | ENSG00000180914 | 615 | NP_000907.2 |
| P2RX4 | ENSG00000135124 | 616 | NP_001243725.1 |
| P2RX5 | ENSG00000083454 | 617 | NP_002552.2 |
| P4HA2 | ENSG00000072682 | 618 | NP_001136071.1 |
| PACS1 | ENSG00000175115 | 619 | NP_060496.2 |
| PAFAH1B1 | ENSG00000007168 | 620 | NP_000421.1 |
| PAH | ENSG00000171759 | 621 | NP_000268.1 |
| PARD3B | ENSG00000116117 | 622 | NP_689739.4 |
| PARK2 | ENSG00000185345 | 623 | NP_004553.2 |
| PAX5 | ENSG00000196092 | 624 | NP_057953.1 |
| PAX6 | ENSG00000007372 | 625 | NP_001245391.1 |
| PCCA | ENSG00000175198 | 626 | NP_000273.2 |
| PCCB | ENSG00000114054 | 627 | NP_001171485.1 |
| PCDH10 | ENSG00000138650 | 628 | NP_116586.1 |
| PCDH15 | ENSG00000150275 | 629 | NP_001136235.1 |
| PCDH19 | ENSG00000165194 | 630 | NP_001171809.1 |
| PCDH8 | ENSG00000136099 | 631 | NP_002581.2 |
| PCDH9 | ENSG00000184226 | 632 | NP_982354.1 |
| PCDHA1 | ENSG00000204970 | 633 | NP_061723.1 |
| PCDHA10 | ENSG00000250120 | 634 | NP_061724.1 |
| PCDHA11 | ENSG00000249158 | 635 | NP_061725.1 |
| PCDHA12 | ENSG00000251664 | 636 | NP_061726.1 |
| PCDHA13 | ENSG00000239389 | 637 | NP_061727.1 |
| PCDHA2 | ENSG00000204969 | 638 | NP_061728.1 |
| PCDHA3 | ENSG00000255408 | 639 | NP_061729.1 |
| PCDHA4 | ENSG00000204967 | 640 | NP_061730.1 |
| PCDHA5 | ENSG00000204965 | 641 | NP_061731.1 |
| PCDHA6 | ENSG00000081842 | 642 | NP_061732.1 |
| PCDHA7 | ENSG00000204963 | 643 | NP_061733.1 |
| PCDHA8 | ENSG00000204962 | 644 | NP_061734.1 |
| PCDHA9 | ENSG00000204961 | 645 | NP_114063.1 |
| PCDHAC1 | ENSG00000248383 | 646 | NP_061721.2 |
| PCDHAC2 | ENSG00000243232 | 647 | NP_061722.1 |
| PCDHGA11 | ENSG00000253873 | 648 | NP_061737.1 |
| PDE1C | ENSG00000154678 | 649 | NP_001177987.1 |
| PDE4A | ENSG00000065989 | 650 | NP_001104777.1 |
| PDE4B | ENSG00000184588 | 651 | NP_002591.2 |
| PDZD4 | ENSG00000067840 | 652 | NP_115901.2 |
| PECR | ENSG00000115425 | 653 | NP_060911.2 |
| PERI | ENSG00000179094 | 654 | NP_002607.2 |
| PEX7 | ENSG00000112357 | 655 | NP_000279.1 |
| PGLYRP2 | ENSG00000161031 | 656 | NP_443122.3 |
| PHF2 | ENSG00000197724 | 657 | NP_005383.3 |
| PHF8 | ENSG00000172943 | 658 | NP_001171825.1 |
| PHIP | ENSG00000146247 | 659 | NP_060404.4 |
| PHRF1 | ENSG00000070047, | 660 | NP_065952.2 |
| | ENSG00000274780 | 661 | |
| PIK3CG | ENSG00000105851 | 662 | NP_002640.2 |
| PIK3R2 | ENSG00000105647 | 663 | NP_005018.1 |
| PINX1 | ENSG00000254093 | 664 | NP_060354.4 |
| PITX1 | ENSG00000069011 | 665 | NP_002644.4 |
| PLAUR | ENS G00000011422 | 666 | NP_002650.1 |
| PLCB1 | ENSG00000182621 | 667 | NP_056007.1 |
| PLCD1 | ENSG00000187091 | 668 | NP_001124436.1 |
| PLN | ENSG00000198523 | 669 | NP_002658.1 |
| PLXNA4 | ENSG00000221866 | 670 | NP_065962.1 |
| PLXNB1 | ENSG00000164050 | 671 | NP_001123554.1 |
| POGZ | ENSG00000143442 | 672 | NP_055915.2 |
| POMGNT1 | ENSG00000085998 | 673 | NP_001230695.1 |
| PON1 | ENSG00000005421 | 674 | NP_000437.3 |
| POT1 | ENSG00000128513 | 675 | NP_056265.2 |
| POU3F2 | ENSG00000184486 | 676 | NP_005595.2 |
| PPFIA1 | ENSG00000131626 | 677 | NP_003617.1 |
| PPP1R1B | ENSG00000131771 | 678 | NP_115568.2 |
| PPP1R3F | ENSG00000049769 | 679 | NP_149992.3 |
| PPP2R1B | ENSG00000137713 | 680 | NP_859050.1 |
| PPP2R5D | ENSG00000112640 | 681 | NP_006236.1 |
| PREX1 | ENSG00000124126 | 682 | NP_065871.2 |
| PRICKLE1 | ENSG00000139174 | 683 | NP_694571.2 |
| PRICKLE2 | ENSG00000163637 | 684 | NP_942559.1 |
| PRKCB | ENSG00000166501 | 685 | NP_002729.2 |
| PRKD1 | ENSG00000184304 | 686 | NP_002733.2 |
| PRKDC | ENSG00000253729 | 687 | NP_008835.5 |
| PRODH | ENSG00000100033 | 688 | NP_057419.4 |
| PRSS38 | ENSG00000185888 | 689 | NP_898885.1 |
| PRUNE2 | ENSG00000106772 | 690 | NP_056040.2 |
| PSD3 | ENSG00000156011 | 691 | NP_056125.3 |
| PSMD10 | ENSG00000101843 | 692 | NP_002805.1 |
| PSMD12 | ENSG00000197170 | 693 | NP_002807.1 |
| PTBP2 | ENSG00000117569 | 694 | NP_067013.1 |
| PTCHD1 | ENSG00000165186 | 695 | NP_775766.2 |
| PTEN | ENSG00000171862 | 696 | NP_000305.3 |
| PTGER3 | ENSG00000050628 | 697 | NP_942011.1 |
| PTGS2 | ENSG00000073756 | 698 | NP_000954.1 |
| PTK7 | ENSG00000112655 | 699 | NP_001257327.1 |
| PTPN11 | ENSG00000179295 | 700 | NP_002825.3 |
| PTPRB | ENSG00000127329 | 701 | NP_001103224.1 |
| PTPRC | ENSG00000262418, | 702 | NP_002829.3 |
| | ENSG00000081237 | 703 | |
| PTPRT | ENSG00000196090 | 704 | NP_573400.3 |
| PVALB | ENSG00000100362, | 705 | NP_002845.1 |
| | ENSG00000274665 | 706 | |
| PXDN | ENSG00000130508 | 707 | NP_036425.1 |
| PYHIN1 | ENSG00000163564 | 708 | NP_689714.2 |
| RAB11FIP5 | ENSG00000135631 | 709 | NP_056285.1 |
| RAB19 | ENSG00000146955 | 710 | NP_001008749.2 |
| RAB2A | ENSG00000104388 | 711 | NP_002856.1 |
| RAB39B | ENSG00000155961 | 712 | NP_741995.1 |
| RAB43 | ENSG00000172780 | 713 | NP_001191812.1 |
| RAI1 | ENSG00000108557 | 714 | NP_109590.3 |
| RANBP17 | ENSG00000204764 | 715 | NP_075048.1 |
| RAPGEF4 | ENSG00000091428 | 716 | NP_008954.2 |
| RASD1 | ENSG00000108551 | 717 | NP_057168.1 |
| RASSF5 | ENSG00000266094 | 718 | NP_872604.1 |
| RB1CC1 | ENSG00000023287 | 719 | NP_055596.3 |
| RBFOX1/ | ENSG00000078328 | 720 | NP_665898.1 |
| A2BP1 | | | |
| RBM27 | ENSG00000091009 | 721 | NP_061862.1 |
| RBM8A | ENSG00000265241 | 722 | NP_005096.1 |
| RBMS3 | ENSG00000144642 | 723 | NP_001003793.1 |
| REEP3 | ENSG00000165476 | 724 | NP_001001330.1 |
| RELN | ENSG00000189056 | 725 | NP_005036.2 |
| RERE | ENSG00000142599 | 726 | NP_036234.3 |
| RFWD2 | ENSG00000143207 | 727 | NP_071902.2 |
| RGS7 | ENSG00000182901 | 728 | NP_002915.3 |
| RHOXF1 | ENSG00000101883 | 729 | NP_644811.1 |
| RIMS1 | ENSG00000079841 | 730 | NP_055804.2 |
| RIMS3 | ENSG00000117016 | 731 | NP_055562.2 |
| RIT2 | ENSG00000152214 | 732 | NP_002921.1 |
| RLIM | ENSG00000131263 | 733 | NP_057204.2 |
| RNF135 | ENSG00000181481 | 734 | NP_115698.3 |
| RNPS1 | ENSG00000205937 | 735 | NP_542161.1 |
| ROBO1 | ENSG00000169855 | 736 | NP_002932.1 |
| ROBO2 | ENSG00000185008 | 737 | NP_001122401.1 |
| RORA | ENSG00000069667 | 738 | NP_599022.1 |
| RPL10 | ENSG00000147403 | 739 | NP_001243506.2 |
| RPP25 | ENSG00000178718 | 740 | NP_060263.2 |
| RPS6KA2 | ENSG00000071242 | 741 | NP_001006933.1 |
| RPS6KA3 | ENSG00000177189 | 742 | NP_004577.1 |
| SAE1 | ENSG00000142230 | 743 | NP_005491.1 |
| SATB2 | ENSG00000119042 | 744 | NP_001165980.1 |
| SBF1 | ENSG00000100241 | 745 | NP_002963.2 |
| SCFD2 | ENSG00000184178 | 746 | NP_689753.2 |
| SCN1A | ENSG00000144285 | 747 | NP_001159435.1 |
| SCN2A | ENSG00000136531 | 748 | NP_066287.2 |
| SCN4A | ENSG00000007314 | 749 | NP_000325.4 |
| SCN5A | ENSG00000183873 | 750 | NP_001092874.1 |
| SCN7A | ENSG00000136546 | 751 | NP_002967.2 |
| SCN8A | ENSG00000196876 | 752 | NP_055006.1 |
| SCN9A | ENSG00000169432 | 753 | NP_002968.1 |
| SDC2 | ENSG00000169439 | 754 | NP_002989.2 |
| SDK1 | ENSG00000146555 | 755 | NP_689957.3 |
| SEMA5A | ENSG00000112902 | 756 | NP_003957.2 |
| SERPINE1 | ENSG00000106366 | 757 | NP_000593.1 |
| SETBP1 | ENSG00000152217 | 758 | NP_056374.2 |
| SETD2 | ENSG00000181555 | 759 | NP_054878.5 |
| SETD5 | ENSG00000168137 | 760 | NP_001073986.1 |
| SETDB1 | ENSG00000143379 | 761 | NP_001138887.1 |
| SETDB2 | ENSG00000136169 | 762 | NP_114121.2 |
| SEZ6L2 | ENSG00000174938 | 763 | NP_001230261.1 |
| SGSH | ENSG00000181523 | 764 | NP_000190.1 |
| SGSM3 | ENSG00000100359 | 765 | NP_056520.2 |
| SH3KBP1 | ENSG00000147010 | 766 | NP_114098.1 |
| SHANK1 | ENSG00000161681 | 767 | NP_057232.2 |
| SHANK2 | ENSG00000162105 | 768 | NP_036441.2 |
| SHANK3 | ENSG00000251322, | 769 | NP_277052.1 |
| | ENSG00000283243 | 770 | |
| SHOX | ENSG00000185960 | 771 | NP_000442.1 |
| SIK1 | ENSG00000142178 | 772 | NP_775490.2 |
| SIN3A | ENSG00000169375 | 773 | NP_001138829.1 |
| SLC12A5 | ENSG00000124140 | 774 | NP_001128243.1 |
| SLC16A3 | ENSG00000141526 | 775 | NP_001035888.1 |
| SLC16A7 | ENSG00000118596 | 776 | NP_004722.2 |
| SLC1A1 | ENSG00000106688 | 777 | NP_004161.4 |
| SLC1A2 | ENSG00000110436 | 778 | NP_004162.2 |
| SLC22A15 | ENSG00000163393 | 779 | NP_060890.2 |
| SLC22A9 | ENSG00000149742 | 780 | NP_543142.2 |
| SLC24A2 | ENSG00000155886 | 781 | NP_065077.1 |
| SLC25A12 | ENSG00000115840 | 782 | NP_003696.2 |
| SLC25A14 | ENSG00000102078 | 783 | NP_003942.1 |
| SLC25A24 | ENSG00000085491, | 784 | NP_037518.3 |
| | ENSG00000284468 | 785 | |
| SLC25A27 | ENSG00000153291 | 786 | NP_004268.3 |
| SLC27A4 | ENSG00000167114 | 787 | NP_005085.2 |
| SLC29A4 | ENSG00000164638 | 788 | NP_694979.2 |
| SLC30A3 | ENSG00000115194 | 789 | NP_003450.2 |
| SLC30A5 | ENSG00000145740 | 790 | NP_075053.2 |
| SLC33A1 | ENSG00000169359 | 791 | NP_001177921.1 |
| SLC35A3 | ENSG00000117620 | 792 | NP_001258614.1 |
| SLC38A10 | ENSG00000157637 | 793 | NP_001033073.1 |
| SLC39A11 | ENSG00000282291, | 794 | NP_001153242.1 |
| | ENSG00000133195 | 795 | |
| SLC4A10 | ENSG00000144290 | 796 | NP_001171486.1 |
| SLC6A1 | ENSG00000157103 | 797 | NP_003033.3 |
| SLC6A3 | ENSG00000276996, | 798 | NP_001035.1 |
| | ENSG00000142319 | 799 | |
| SLC6A4 | ENSG00000108576 | 800 | NP_001036.1 |
| SLC6A8 | ENSG00000130821 | 801 | NP_005620.1 |
| SLC7A3 | ENSG00000165349 | 802 | NP_116192.4 |
| SLC7A5 | ENSG00000103257 | 803 | NP_003477.4 |
| SLC9A6 | ENSG00000198689 | 804 | NP_001036002.1 |
| SLC9A9 | ENSG00000181804 | 805 | NP_775924.1 |
| SLCO1B3 | ENSG00000111700 | 806 | NP_062818.1 |
| SLIT3 | ENSG00000184347 | 807 | NP_003053.1 |
| SLITRK5 | ENSG00000165300 | 808 | NP_056382.1 |
| SMARCA2 | ENSG00000080503 | 809 | NP_003061.3 |
| SMARCC2 | ENSG00000139613 | 810 | NP_003066.2 |
| SMC3 | ENSG00000108055 | 811 | NP_005436.1 |
| SMG6 | ENSG00000070366 | 812 | NP_060045.4 |
| SNAP25 | ENSG00000132639 | 813 | NP_003072.2 |
| SND1 | ENSG00000197157 | 814 | NP_055205.2 |
| SNRPN | ENSG00000128739 | 815 | NP_073716.1 |
| SNTG2 | ENSG00000172554, | 816 | NP_061841.2 |
| | ENSG00000281486, | 817 | |
| | ENSG00000281020 | 818 | |
| SNX14 | ENSG00000135317 | 819 | NP_722523.1 |
| SNX19 | ENSG00000120451 | 820 | NP_055573.2 |
| SOD1 | ENSG00000142168 | 821 | NP_000445.1 |
| SOX5 | ENSG00000134532 | 822 | NP_008871.3 |
| SPARCL1 | ENSG00000152583 | 823 | NP_001121782.1 |
| SPAST | ENSG00000021574 | 824 | NP_055761.2 |
| SRCAP | ENSG00000080603 | 825 | NP_006653.2 |
| SRD5A2 | ENSG00000277893 | 826 | NP_000339.2 |
| SRGAP3 | ENSG00000196220 | 827 | NP_055665.1 |
| SRRM4 | ENSG00000139767 | 828 | NP_919262.2 |
| SSPO | ENSG00000197558 | 829 | NP_940857.2 |
| ST7 | ENSG00000004866 | 830 | NP_068708.1 |
| ST8SIA2 | ENSG00000140557 | 831 | NP_006002.1 |
| STAG1 | ENSG00000118007 | 832 | NP_005853.2 |
| STAT1 | ENSG00000115415 | 833 | NP_009330.1 |
| STK39 | ENSG00000198648 | 834 | NP_037365.2 |
| STX1A | ENSG00000106089 | 835 | NP_004594.1 |
| STXBP1 | ENSG00000136854 | 836 | NP_003156.1 |
| STXBP5 | ENSG00000164506 | 837 | NP_001121187.1 |
| STYK1 | ENSG00000060140 | 838 | NP_060893.2 |
| SUCLG2 | ENSG00000172340 | 839 | NP_001171070.1 |
| SYAP1 | ENSG00000169895 | 840 | NP_116185.2 |
| SYN1 | ENSG00000008056 | 841 | NP_008881.2 |
| SYN2 | ENSG00000157152 | 842 | NP_598328.1 |
| SYN3 | ENSG00000185666 | 843 | NP_003481.3 |
| SYNE1 | ENSG00000131018 | 844 | NP_892006.3 |
| SYNGAP1 | ENSG00000197283, | 845 | NP_006763.2 |
| | ENSG00000227460 | 846 | |
| SYT17 | ENSG00000103528 | 847 | NP_057608.2 |
| SYT3 | ENSG00000213023 | 848 | NP_115674.1 |
| TAF1 | ENSG00000147133 | 849 | NP_004597.2 |
| TAF1C | ENSG00000103168 | 850 | NP_005670.3 |
| TAF1L | ENSG00000122728 | 851 | NP_722516.1 |
| TANC2 | ENSG00000170921 | 852 | NP_079461.2 |
| TBC1D31 | ENSG00000156787 | 853 | NP_663622.2 |
| TBC1D5 | ENSG00000131374 | 854 | NP_001127853.1 |
| TBC1D7 | ENSG00000145979 | 855 | NP_001137436.1 |
| TBL1X | ENSG00000101849 | 856 | NP_001132938.1 |
| TBL1XR1 | ENSG00000177565 | 857 | NP_078941.2 |
| TBR1 | ENSG00000136535 | 858 | NP_006584.1 |
| TBX1 | ENSG00000184058 | 859 | NP_542378.1 |
| TCF20 | ENSG00000276461, | 860 | NP_005641.1 |
| | ENSG00000262024, | 861 | |
| | ENSG00000100207, | 862 | |
| | ENSG00000280467, | 863 | |
| | ENSG00000281897, | 864 | |
| | ENSG00000283026, | 865 | |
| | ENSG00000282892, | 866 | |
| | ENSG00000283681 | 867 | |
| TCF4 | ENSG00000196628 | 868 | NP_001230155.2 |
| TCF7L2 | ENSG00000148737 | 869 | NP_001139746.1 |
| TDO2 | ENSG00000151790, | 870 | NP_005642.1 |
| | ENSG00000262635 | 871 | |
| TERF2 | ENSG00000132604 | 872 | NP_005643.2 |
| TERT | ENSG00000164362 | 873 | NP_937983.2 |
| TET2 | ENSG00000168769 | 874 | NP_001120680.1 |
| TGM3 | ENSG00000125780 | 875 | NP_003236.3 |
| TH | ENSG00000180176 | 876 | NP_954986.2 |
| THAP8 | ENSG00000161277 | 877 | NP_689871.1 |
| THBS1 | ENSG00000137801 | 878 | NP_003237.2 |
| THRA | ENSG00000126351 | 879 | NP_003241.2 |
| TLK2 | ENSG00000146872 | 880 | NP_006843.2 |
| TM4SF20 | ENSG00000168955 | 881 | NP_079071.2 |
| TMEM231 | ENSG00000205084 | 882 | NP_001070884.1 |
| TMLHE | ENSG00000185973 | 883 | NP_060666.1 |
| TNIP2 | ENSG00000168884 | 884 | NP_077285.3 |
| TNN | ENSG00000120332 | 885 | NP_071376.1 |
| TNRC6B | ENSG00000100354 | 886 | NP_001155973.1 |
| TOMM20 | ENSG00000173726 | 887 | NP_055580.1 |
| TOP1 | ENSG00000198900 | 888 | NP_003277.1 |
| TOP3B | ENSG00000100038 | 889 | NP_003926.1 |
| TPH2 | ENSG00000139287 | 890 | NP_775489.2 |
| TPO | ENSG00000115705, | 891 | NP_000538.3 |
| | ENSG00000277603 | 892 | |
| TRIM33 | ENSG00000197323 | 893 | NP_056990.3 |
| TRIO | ENSG00000038382 | 894 | NP_009049.2 |
| TRIP12 | ENSG00000153827 | 895 | NP_004229.1 |
| TRPC6 | ENSG00000137672 | 896 | NP_004612.2 |
| TRPM1 | ENSG00000134160, | 897 | NP_001238949.1 |
| | ENSG00000274965 | 898 | |
| TSC1 | ENSG00000165699 | 899 | NP_000359.1 |
| TSC2 | ENSG00000103197 | 900 | NP_000539.2 |
| TSHZ3 | ENSG00000121297 | 901 | NP_065907.2 |
| TSN | ENSG00000211460 | 902 | NP_004613.1 |
| TSPAN7 | ENSG00000156298 | 903 | NP_004606.2 |
| TTI2 | ENSG00000129696 | 904 | NP_079391.2 |
| TTN | ENSG00000155657 | 905 | NP_001254479.1 |
| TUBGCP5 | ENSG00000280807, | 906 | NP_001096080.1 |
| | ENSG00000275835, | 907 | |
| | ENSG00000276856 | 908 | |
| TYR | ENSG00000077498 | 909 | NP_000363.1 |
| UBA6 | ENSG00000033178 | 910 | NP_060697.4 |
| UBE2H | ENSG00000186591 | 911 | NP_003335.1 |
| UBE3A | ENSG00000114062 | 912 | NP_000453.2 |
| UBE3B | ENSG00000151148 | 913 | NP_904324.1 |
| UBE3C | ENSG00000009335 | 914 | NP_055486.2 |
| UBL7 | ENSG00000138629 | 915 | NP_116296.1 |
| UBR5 | ENSG00000104517 | 916 | NP_056986.2 |
| UBR7 | ENSG00000278787, | 917 | NP_786924.2 |
| | ENSG00000012963 | 918 | |
| UCN3 | ENSG00000178473 | 919 | NP_444277.2 |
| UNC13A | ENSG00000130477 | 920 | NP_001073890.2 |
| UNC80 | ENSG00000144406 | 921 | NP_115893.1 |
| UPB1 | ENSG00000100024 | 922 | NP_057411.1 |
| UPF2 | ENSG00000151461 | 923 | NP_056357.1 |
| UPF3B | ENSG00000125351 | 924 | NP_542199.1 |
| USP45 | ENSG00000123552 | 925 | NP_001073950.1 |
| USP7 | ENSG00000187555 | 926 | NP_003461.2 |
| USP9Y | ENSG00000114374 | 927 | NP_004645.2 |
| UTRN | ENSG00000152818 | 928 | NP_009055.2 |
| VASH1 | ENSG00000071246 | 929 | NP_055724.1 |
| VIL1 | ENSG00000127831 | 930 | NP_009058.2 |
| VIP | ENSG00000146469 | 931 | NP_003372.1 |
| VLDLR | ENSG00000147852 | 932 | NP_003374.3 |
| VPS13B | ENSG00000132549 | 933 | NP_060360.3 |
| VSIG4 | ENSG00000155659 | 934 | NP_009199.1 |
| WAC | ENSG00000095787 | 935 | NP_057712.2 |
| WDFY3 | ENSG00000163625 | 936 | NP_055806.2 |
| WDR93 | ENSG00000140527 | 937 | NP_064597.1 |
| WNK3 | ENSG00000196632 | 938 | NP_065973.2 |
| WNT1 | ENSG00000125084 | 939 | NP_005421.1 |
| WNT2 | ENSG00000105989 | 940 | NP_003382.1 |
| WWOX | ENSG00000186153 | 941 | NP_057457.1 |
| XIRP1 | ENSG00000168334 | 942 | NP_919269.2 |
| XPC | ENSG00000154767 | 943 | NP_004619.3 |
| XPO1 | ENSG00000082898 | 944 | NP_003391.1 |
| YEATS2 | ENSG00000163872 | 945 | NP_060493.3 |
| YTHDC2 | ENSG00000047188 | 946 | NP_073739.3 |
| YWHAE | ENSG00000108953, | 947 | NP_006752.1 |
| | ENSG00000274474 | 948 | |
| ZBTB16 | ENSG00000109906 | 949 | NP_005997.2 |
| ZBTB20 | ENSG00000181722 | 950 | NP_001157814.1 |
| ZC3H4 | ENSG00000130749 | 951 | NP_055983.1 |
| ZMYND11 | ENSG00000015171 | 952 | NP_006615.2 |
| ZNF18 | ENSG00000154957 | 953 | NP_653281.2 |
| ZNF292 | ENSG00000188994 | 954 | NP_055836.1 |
| ZNF385B | ENSG00000144331 | 955 | NP_689733.3 |
| ZNF407 | ENSG00000215421 | 956 | NP_060227.2 |
| ZNF462 | ENSG00000148143 | 957 | NP_067047.4 |
| ZNF517 | ENSG00000197363 | 958 | NP_998770.2 |
| ZNF559 | ENSG00000188321 | 959 | NP_001189335.1 |
| ZNF713 | ENSG00000178665 | 960 | NP_872439.1 |
| ZNF774 | ENSG00000196391 | 961 | NP_001004309.2 |
| ZNF8 | ENSG00000278129 | 962 | NP_066575.2 |
| ZNF804A | ENSG00000170396 | 963 | NP_919226.1 |
| ZNF827 | ENSG00000151612 | 964 | NP_849157.2 |
| ZSWIM5 | ENSG00000162415 | 965 | NP_065934.1 |
| ZWILCH | ENSG00000174442 | 966 | NP_060445.3 |

*Representative protein in the family encoded by the gene.

TABLE 2
(338 Genes)
SFARI genes that have a high-scoring hnRNP L motif within
500 bp of one of the Castle splice sites.

| | | | | Genes with hnRNP |
|---|---|---|---|---|
| | | | | binding sites within |
| | | | | 200 base pairs of an |
| | | SEQ | Accession | RBFox1/A2BP1 |
| Human Gene | Ensembl Identifier | ID NO | Number* | binding site |
| A2BP1 | ENSG00000078328 | 967 | NP_665898.1 | X |
| ABAT | ENSG00000183044 | 1 | NP_001120920.1 | |
| ABCA10 | ENSG00000154263 | 2 | NP_525021.3 | |
| ABCA7 | ENSG00000064687 | 3 | NP_061985.2 | X |
| ACHE | ENSG00000087085 | 5 | NP_000656.1 | |
| ACTN4 | ENSG00000130402, | 6 | NP_004915.2 | |
| | ENSG00000282844 | 7 | | |
| ACY1 | ENSG00000243989 | 8 | NP_001185824.1 | |
| ADAMTS18 | ENSG00000140873 | 10 | NP_955387.1 | X |
| ADARB1 | ENSG00000197381 | 11 | NP_056648.1 | |
| ADK | ENSG00000156110 | 13 | NP_001114.2 | |
| ADNP | ENSG00000101126 | 14 | NP_056154.1 | X |
| ADSL | ENSG00000239900 | 18 | NP_000017.1 | X |
| AHI1 | ENSG00000135541 | 26 | NP_001128302.1 | X |
| AKAP9 | ENSG00000127914 | 27 | NP_005742.4 | |
| ALG6 | ENSG00000088035 | 30 | NP_037471.2 | |
| AMT | ENSG00000145020 | 33 | NP_000472.2 | |
| ANK2 | ENSG00000145362 | 34 | NP_001139.3 | |
| ANK3 | ENSG00000151150 | 35 | NP_066267.2 | X |
| ANKRD11 | ENSG00000167522 | 36 | NP_001243111.1 | |
| ANXA1 | ENSG00000135046 | 38 | NP_000691.1 | |
| AP1S2 | ENSG00000182287 | 39 | NP_003907.3 | |
| APC | ENSG00000134982 | 42 | NP_001120982.1 | |
| APP | ENSG00000142192 | 44 | NP_000475.1 | X |
| ARHGAP15 | ENSG00000075884 | 47 | NP_060930.3 | |
| ARHGEF9 | ENSG00000131089 | 51 | NP_056000.1 | |
| ARID1B | ENSG00000049618 | 52 | NP_065783.3 | |
| ARNT2 | ENSG00000172379 | 53 | NP_055677.3 | |
| ASMT | ENSG00000196433 | 56 | NP_004034.2 | X |
| ASTN2 | ENSG00000148219 | 58 | NP_054729.3 | |
| ATP10A | ENSG00000206190 | 61 | NP_077816.1 | |
| ATP2B2 | ENSG00000157087 | 63 | NP_001001331.1 | X |
| ATP8A1 | ENSG00000124406 | 64 | NP_006086.1 | |
| ATRNL1 | ENSG00000107518 | 65 | NP_997186.1 | X |
| ATRX | ENSG00000085224 | 66 | NP_000480.3 | X |
| AUTS2 | ENSG00000158321 | 68 | NP_056385.1 | |
| BAIAP2 | ENSG00000175866 | 72 | NP_059345.1 | X |
| BCAS1 | ENSG00000064787 | 74 | NP_003648.2 | X |
| BCKDK | ENSG00000103507 | 75 | NP_005872.2 | |
| BCL11A | ENSG00000119866 | 76 | NP_075044.2 | X |
| BIN1 | ENSG00000136717 | 79 | NP_647593.1 | X |
| BRCA2 | ENSG00000139618 | 82 | NP_000050.2 | |
| BTAF1 | ENSG00000095564 | 84 | NP_003963.1 | |
| CACNA1B | ENSG00000148408 | 97 | NP_000709.1 | |
| CACNA1C | ENSG00000151067 | 98 | NP_955630.3 | X |
| CACNA1D | ENSG00000157388 | 99 | NP_000711.1 | X |
| CACNA1E | ENSG00000198216 | 100 | NP_001192222.1 | |
| CACNA1F | ENSG00000102001 | 101 | NP_005174.2 | |
| CACNA1G | ENSG00000006283 | 102 | NP_061496.2 | |
| CACNA11 | ENSG00000100346 | 104 | NP_066919.2 | |
| CACNA2D3 | ENSG00000157445 | 105 | NP_060868.2 | |
| CACNB2 | ENSG00000165995 | 106 | NP_963890.2 | X |
| CADPS2 | ENSG00000081803 | 109 | NP_001161412.1 | X |
| CAMTAI | ENSG00000171735 | 113 | NP_056030.1 | X |
| CAPN12 | ENSG00000182472 | 114 | NP_653292.2 | |
| CASK | ENSG00000147044 | 117 | NP_003679.2 | X |
| CBS | ENSG00000160200 | 118 | NP_001171479.1 | |
| CCT4 | ENSG00000115484 | 123 | NP_006421.2 | |
| CD38 | ENSG00000004468 | 124 | NP_001766.2 | |
| CD44 | ENSG00000026508 | 125 | NP_000601.3 | X |
| CD99L2 | ENSG00000102181 | 126 | NP_001229543.1 | X |
| CDH11 | ENSG00000140937 | 129 | NP_001788.2 | |
| CEP290 | ENSG00000198707 | 138 | NP_079390.3 | |
| CHD2 | ENSG00000173575 | 141 | NP_001262.3 | X |
| CHD7 | ENSG00000171316 | 142 | NP_060250.2 | |
| CHKB | ENSG00000100288 | 144 | NP_001164100.1 | |
| CHRNA7 | ENSG00000175344 | 146 | NP_001177384.1 | |
| CIB2 | ENSG00000136425 | 149 | NP_006374.1 | |
| CLSTN3 | ENSG00000139182 | 152 | NP_055533.2 | |
| CLTCL1 | ENSG00000070371 | 153 | NP_009029.3 | |
| CMIP | ENSG00000153815 | 154 | NP_938204.2 | |
| CNKSR2 | ENSG00000149970 | 155 | NP_055742.2 | X |
| CNOT3 | ENSG00000277600, | 156 | NP_055331.1 | |
| | ENSG00000274616, | 157 | | |
| | ENSG00000277114, | 158 | | |
| | ENSG00000088038, | 159 | | |
| | ENSG00000273943, | 160 | | |
| | ENSG00000274941, | 161 | | |
| | ENSG00000274176, | 162 | | |
| | ENSG00000277615, | 163 | | |
| | ENSG00000276082, | 164 | | |
| | ENSG00000275979 | 165 | | |
| CNR1 | ENSG00000118432 | 166 | NP_001153698.1 | |
| CNTN5 | ENSG00000149972 | 170 | NP_001230199.1 | |
| CNTNAP3 | ENSG00000106714 | 174 | NP_387504.2 | X |
| CNTNAP5 | ENSG00000155052 | 176 | NP_570129.1 | |
| CRHR2 | ENSG00000106113 | 178 | NP_001189404.1 | |
| CSNK1D | ENSG00000141551 | 180 | NP_001884.2 | X |
| CYLC2 | ENSG00000155833 | 193 | NP_001331.1 | |
| DAB1 | ENSG00000173406 | 195 | NP_066566.3 | |
| DAPK1 | ENSG00000196730 | 197 | NP_004929.2 | X |
| DAPP1 | ENSG00000070190 | 198 | NP_055210.2 | X |
| DDC | ENSG00000132437 | 202 | NP_001076440.1 | X |
| DDX11 | ENSG00000013573 | 203 | NP_689651.1 | |
| DDX3X | ENSG00000215301 | 204 | NP_001347.3 | |
| DEAF1 | ENSG00000177030, | 206 | NP_066288.2 | |
| | ENSG00000282712 | 207 | | |
| DENR | ENSG00000139726 | 208 | NP_003668.2 | X |
| DGKZ | ENSG00000149091 | 211 | NP_001099010.1 | |
| DHCR7 | ENSG00000172893 | 212 | NP_001351.2 | |
| DLG1 | ENSG00000075711 | 217 | NP_004078.2 | |
| DLGAP2 | ENSG00000198010, | 220 | NP_004736.2 | |
| | ENSG00000282152, | 221 | | |
| | ENSG00000274161, | 222 | | |
| | ENSG00000282318 | 223 | | |
| DMD | ENSG00000198947 | 228 | NP_003997.1 | X |
| DMXL2 | ENSG00000104093 | 230 | NP_001167587.1 | X |
| DNM1L | ENSG00000087470 | 235 | NP_036192.2 | X |
| DNMT3A | ENSG00000119772 | 236 | NP_072046.2 | |
| DOCK4 | ENSG00000128512 | 239 | NP_055520.3 | |
| DOCK8 | ENSG00000107099 | 240 | NP_982272.2 | |
| DPP4 | ENSG00000197635 | 243 | NP_001926.2 | X |
| DRD2 | ENSG00000149295 | 248 | NP_000786.1 | |
| DSCAM | ENSG00000171587 | 250 | NP_001380.2 | |
| DUSP15 | ENSG00000149599 | 252 | NP_542178.2 | X |
| DUSP22 | ENSG00000112679 | 253 | NP_064570.1 | |
| DYRK1A | ENSG00000157540 | 258 | NP_001387.2 | X |
| EGR2 | ENSG00000122877 | 261 | NP_001129649.1 | X |
| EIF4E | ENSG00000151247 | 264 | NP_001124151.1 | |
| ELAVL2 | ENSG00000107105 | 266 | NP_004423.2 | |
| ELP4 | ENSG00000109911 | 268 | NP_061913.3 | X |
| EPHB2 | ENSG00000133216 | 275 | NP_004433.2 | X |
| ERG | ENSG00000157554 | 281 | NP_001129626.1 | |
| ESR1 | ENSG00000091831 | 283 | NP_001116212.1 | |
| ESR2 | ENSG00000140009 | 284 | NP_001428.1 | X |
| FBN1 | ENSG00000166147 | 301 | NP_000129.3 | X |
| FLT1 | ENSG00000102755 | 311 | NP_002010.2 | |
| FMRI | ENSG00000102081 | 312 | NP_002015.1 | |
| FOLH1 | ENSG00000086205 | 313 | NP_004467.1 | |
| FOXP1 | ENSG00000114861 | 315 | NP_001231739.1 | X |
| FOXP2 | ENSG00000128573 | 316 | NP_683696.2 | |
| FRK | ENSG00000111816 | 317 | NP_002022.1 | |
| GABRB3 | ENSG00000166206 | 324 | NP_068712.1 | |
| GALNT14 | ENSG00000158089 | 329 | NP_001240755.1 | |
| GAP43 | ENSG00000172020 | 331 | NP_001123536.1 | |
| GAS2 | ENSG00000148935 | 332 | NP_001137302.1 | |
| GDA | ENSG00000119125 | 334 | NP_001229434.1 | X |
| GNAS | ENSG00000087460 | 341 | NP_536350.2 | X |
| GNB1L | ENSG00000185838 | 342 | NP_443730.1 | X |
| GPD2 | ENSG00000115159 | 345 | NP_001076581.2 | X |
| GRID1 | ENSG00000182771 | 351 | NP_060021.1 | |
| GRIK2 | ENSG00000164418 | 354 | NP_068775.1 | |
| GRIN1 | ENSG00000176884 | 358 | NP_001172019.1 | X |
| GRM1 | ENSG00000152822 | 362 | NP_000829.2 | X |
| GRM4 | ENSG00000124493 | 363 | NP_000832.1 | |
| GRM5 | ENSG00000168959 | 364 | NP_001137303.1 | X |
| GRM7 | ENSG00000196277 | 365 | NP_870989.1 | X |
| GRM8 | ENSG00000179603 | 366 | NP_000836.2 | X |
| GSK3B | ENSG00000082701 | 368 | NP_002084.2 | |
| GSN | ENSG00000148180 | 369 | NP_000168.1 | |
| GSTM1 | ENSG00000134184 | 370 | NP_000552.2 | |
| GTF2I | ENSG00000263001 | 371 | NP_127492.1 | |
| GUCY1A2 | ENSG00000152402 | 372 | NP_001243353.1 | |
| HDAC3 | ENSG00000171720 | 375 | NP_003874.2 | X |
| HDAC4 | ENSG00000068024 | 376 | NP_006028.2 | |
| HDAC6 | ENSG00000094631 | 377 | NP_006035.2 | |
| HLA-DRB1 | ENSG00000236884, | 399 | NP_001230894.1 | X |
| | ENSG00000228080, | 400 | | |
| | ENSG00000206240, | 401 | | |
| | ENSG00000229074, | 402 | | |
| | ENSG00000206306, | 403 | | |
| | ENSG00000196126 | 404 | | |
| HMGN1 | ENSG00000205581 | 405 | NP_004956.5 | |
| HOXA1 | ENSG00000105991 | 408 | NP_005513.1 | |
| HRAS | ENSG00000174775, | 410 | NP_001123914.1 | |
| | ENSG00000276536 | 411 | | |
| HTR3A | ENSG00000166736 | 416 | NP_998786.2 | X |
| HTR7 | ENSG00000148680 | 418 | NP_062873.1 | |
| ICA1 | ENSG00000003147 | 422 | NP_071682.1 | |
| IL16 | ENSG00000172349 | 425 | NP_757366.2 | |
| IQSEC2 | ENSG00000124313 | 437 | NP_001104595.1 | X |
| ITGA4 | ENSG00000115232 | 439 | NP_000876.3 | |
| ITPR1 | ENSG00000150995 | 442 | NP_001161744.1 | X |
| JMJD1C | ENSG00000171988 | 445 | NP_116165.1 | X |
| KCNMA1 | ENSG00000156113 | 455 | NP_001154824.1 | |
| KCNQ2 | ENSG00000281151, | 456 | NP_742105.1 | X |
| | ENSG00000075043 | 457 | | |
| KCNT1 | ENSG00000107147 | 459 | NP_065873.2 | |
| KHDRBS3 | ENSG00000131773 | 466 | NP_006549.1 | X |
| KIF13B | ENSG00000197892 | 469 | NP_056069.2 | X |
| KIF5C | ENSG00000276734, | 470 | NP_004513.1 | |
| | ENSG00000168280 | 471 | | |
| KLC2 | ENSG00000174996 | 474 | NP_073733.1 | X |
| KMO | ENSG00000117009 | 475 | NP_003670.2 | X |
| LAMC3 | ENSG00000050555 | 484 | NP_006050.3 | |
| LEP | ENSG00000174697 | 485 | NP_000221.1 | |
| LIN7B | ENSG00000104863 | 486 | NP_071448.1 | |
| LPL | ENSG00000175445 | 488 | NP_000228.1 | X |
| LRBA | ENSG00000198589 | 489 | NP_006717.2 | X |
| LRFN5 | ENSG00000165379 | 490 | NP_689660.2 | |
| LRRC7 | ENSG00000033122 | 495 | NP_065845.1 | X |
| LZTR1 | ENSG00000099949 | 496 | NP_006758.2 | |
| MAGED1 | ENSG00000179222 | 499 | NP_001005333.1 | |
| MAL | ENSG00000172005 | 501 | NP_002362.1 | |
| MAP2 | ENSG00000078018 | 504 | NP_002365.3 | X |
| MAPK3 | ENSG00000102882 | 507 | NP_002737.2 | |
| MARK1 | ENSG00000116141 | 509 | NP_061120.3 | |
| MBD1 | ENSG00000141644 | 510 | NP_001191065.1 | X |
| MBD4 | ENSG00000129071 | 512 | NP_003916.1 | |
| MBD5 | ENSG00000204406 | 513 | NP_060798.2 | |
| MBD6 | ENSG00000166987 | 514 | NP_443129.3 | |
| MCC | ENSG00000171444 | 526 | NP_001078846.1 | X |
| MED12 | ENSG00000184634 | 531 | NP_005111.2 | |
| MEF2C | ENSG00000081189 | 533 | NP_001180276.1 | X |
| MET | ENSG00000105976 | 534 | NP_001120972.1 | |
| MNT | ENSG00000070444 | 538 | NP_064706.1 | |
| MPP6 | ENSG00000105926 | 540 | NP_057531.2 | |
| MSR1 | ENSG00000038945 | 543 | NP_619729.1 | |
| MTX2 | ENSG00000128654 | 548 | NP_006545.1 | |
| MYT1L | ENSG00000186487 | 555 | NP_055840.2 | X |
| NAV2 | ENSG00000166833 | 559 | NP_001231892.1 | X |
| NBEA | ENSG00000172915 | 560 | NP_056493.3 | X |
| NCOR1 | ENSG00000141027 | 564 | NP_006302.2 | X |
| NDUFA5 | ENSG00000128609 | 567 | NP_004991.1 | |
| NF1 | ENSG00000196712 | 571 | NP_001035957.1 | |
| NFIA | ENSG00000162599 | 572 | NP_001138984.1 | X |
| NFIX | ENSG00000008441 | 573 | NP_001257972.1 | X |
| NIPA2 | ENSG00000140157 | 576 | NP_001171818.1 | |
| NIPBL | ENSG00000164190 | 577 | NP_597677.2 | |
| NLGN3 | ENSG00000196338 | 581 | NP_851820.1 | |
| NLGN4X | ENSG00000146938 | 582 | NP_065793.1 | X |
| NLGN4Y | ENSG00000165246 | 583 | NP_055708.3 | X |
| NPAS2 | ENSG00000170485 | 587 | NP_002509.2 | |
| NRCAM | ENSG00000091129 | 590 | NP_001032209.1 | X |
| NRG1 | ENSG00000157168 | 591 | NP_039250.2 | X |
| NRXN1 | ENSG00000179915 | 593 | NP_001129131.1 | X |
| NRXN2 | ENSG00000110076 | 594 | NP_055895.1 | X |
| NRXN3 | ENSG00000021645 | 595 | NP_004787.2 | X |
| NSD1 | ENSG00000165671 | 596 | NP_071900.2 | |
| NTRK1 | ENSG00000198400 | 598 | NP_002520.2 | |
| NTRK3 | ENSG00000140538 | 599 | NP_001012338.1 | X |
| NXF5 | ENSG00000126952 | 602 | NP_116564.2 | X |
| OCRL | ENSG00000122126 | 604 | NP_000267.2 | X |
| OGT | ENSG00000147162 | 606 | NP_858058.1 | |
| P4HA2 | ENSG00000072682 | 618 | NP_001136071.1 | |
| PACS1 | ENSG00000175115 | 619 | NP_060496.2 | |
| PAH | ENSG00000171759 | 621 | NP_000268.1 | X |
| PAX5 | ENSG00000196092 | 624 | NP_057953.1 | |
| PAX6 | ENSG00000007372 | 625 | NP_001245391.1 | X |
| PCCA | ENSG00000175198 | 626 | NP_000273.2 | X |
| PCCB | ENSG00000114054 | 627 | NP_001171485.1 | |
| PCDH19 | ENSG00000165194 | 630 | NP_001171809.1 | |
| PCDH8 | ENSG00000136099 | 631 | NP_002581.2 | |
| PECR | ENSG00000115425 | 653 | NP_060911.2 | |
| PER1 | ENSG00000179094 | 654 | NP_002607.2 | X |
| PHF2 | ENSG00000197724 | 657 | NP_005383.3 | |
| PHF8 | ENSG00000172943 | 658 | NP_001171825.1 | |
| PHIP | ENSG00000146247 | 659 | NP_060404.4 | X |
| PIK3R2 | ENSG00000105647 | 663 | NP_005018.1 | |
| PINX1 | ENSG00000254093 | 664 | NP_060354.4 | |
| PLAUR | ENSG00000011422 | 666 | NP_002650.1 | X |
| PLCB1 | ENSG00000182621 | 667 | NP_056007.1 | |
| PLXNB1 | ENSG00000164050 | 671 | NP_001123554.1 | |
| POGZ | ENSG00000143442 | 672 | NP_055915.2 | |
| PPFIA1 | ENSG00000131626 | 677 | NP_003617.1 | X |
| PPP1R3F | ENSG00000049769 | 679 | NP_149992.3 | |
| PREX1 | ENSG00000124126 | 682 | NP_065871.2 | X |
| PRKDC | ENSG00000253729 | 687 | NP_008835.5 | X |
| PRODH | ENSG00000100033 | 688 | NP_057419.4 | X |
| PSD3 | ENSG00000156011 | 691 | NP_056125.3 | |
| PSMD10 | ENSG00000101843 | 692 | NP_002805.1 | |
| PSMD12 | ENSG00000197170 | 693 | NP_002807.1 | |
| PTBP2 | ENSG00000117569 | 694 | NP_067013.1 | X |
| PTGER3 | ENSG00000050628 | 697 | NP_942011.1 | X |
| PTK7 | ENSG00000112655 | 699 | NP_001257327.1 | |
| PTPRC | ENSG00000262418, | 702 | NP_002829.3 | X |
| | ENSG00000081237 | 703 | | |
| PVALB | ENSG00000100362, | 705 | NP_002845.1 | |
| | ENSG00000274665 | 706 | | |
| RANBP17 | ENSG00000204764 | 715 | NP_075048.1 | X |
| RBM27 | ENSG00000091009 | 721 | NP_061862.1 | |
| RBM8A | ENSG00000265241 | 722 | NP_005096.1 | |
| RERE | ENSG00000142599 | 726 | NP_036234.3 | |
| RFWD2 | ENSG00000143207 | 727 | NP_071902.2 | X |
| RGS7 | ENSG00000182901 | 728 | NP_002915.3 | X |
| RIMS1 | ENSG00000079841 | 730 | NP_055804.2 | X |
| RIMS3 | ENSG00000117016 | 731 | NP_055562.2 | |
| RIT2 | ENSG00000152214 | 732 | NP_002921.1 | |
| RNF135 | ENSG00000181481 | 734 | NP_115698.3 | |
| RNPS1 | ENSG00000205937 | 735 | NP_542161.1 | |
| RORA | ENSG00000069667 | 738 | NP_599022.1 | X |
| RPL10 | ENSG00000147403 | 739 | NP_001243506.2 | |
| SAE1 | ENSG00000142230 | 743 | NP_005491.1 | |
| SBF1 | ENSG00000100241 | 745 | NP_002963.2 | |
| SCFD2 | ENSG00000184178 | 746 | NP_689753.2 | X |
| SCN8A | ENSG00000196876 | 752 | NP_055006.1 | |
| SCN9A | ENSG00000169432 | 753 | NP_002968.1 | X |
| SDK1 | ENSG00000146555 | 755 | NP_689957.3 | |
| SETDB1 | ENSG00000143379 | 758 | NP_001138887.1 | |
| SEZ6L2 | ENSG00000174938 | 763 | NP_001230261.1 | |
| SGSH | ENSG00000181523 | 764 | NP_000190.1 | |
| SHANK2 | ENSG00000162105 | 768 | NP_036441.2 | X |
| SHANK3 | ENSG00000251322, | 769 | NP_277052.1 | X |
| | ENSG00000283243 | 770 | | |
| SIN3A | ENSG00000169375 | 773 | NP_001138829.1 | X |
| SLC16A7 | ENSG00000118596 | 776 | NP_004722.2 | |
| SLC22A9 | ENSG00000149742 | 780 | NP_543142.2 | |
| SLC24A2 | ENSG00000155886 | 781 | NP_065077.1 | X |
| SLC25A12 | ENSG00000115840 | 782 | NP_003696.2 | |
| SLC25A14 | ENSG00000102078 | 783 | NP_003942.1 | |
| SLC25A27 | ENSG00000153291 | 786 | NP_004268.3 | X |
| SLC30A5 | ENSG00000145740 | 790 | NP_075053.2 | |
| SLC33A1 | ENSG00000169359 | 791 | NP_001177921.1 | X |
| SLC7A3 | ENSG00000165349 | 802 | NP_116192.4 | |
| SMARCA2 | ENSG00000080503 | 809 | NP_003061.3 | |
| SMARCC2 | ENSG00000139613 | 810 | NP_003066.2 | |
| SNAP25 | ENSG00000132639 | 813 | NP_003072.2 | |
| SNRPN | ENSG00000128739 | 815 | NP_073716.1 | X |
| SNX14 | ENSG00000135317 | 819 | NP_722523.1 | X |
| SOD1 | ENSG00000142168 | 821 | NP_000445.1 | |
| SOX5 | ENSG00000134532 | 822 | NP_008871.3 | |
| SPAST | ENSG00000021574 | 824 | NP_055761.2 | |
| SRCAP | ENSG00000080603 | 825 | NP_006653.2 | |
| SRGAP3 | ENSG00000196220 | 827 | NP_055665.1 | |
| ST7 | ENSG00000004866 | 830 | NP_068708.1 | X |
| ST8SIA2 | ENSG00000140557 | 831 | NP_006002.1 | |
| STXBP1 | ENSG00000136854 | 836 | NP_003156.1 | X |
| STXBP5 | ENSG00000164506 | 837 | NP_001121187.1 | |
| SUCLG2 | ENSG00000172340 | 839 | NP_001171070.1 | |
| SYN2 | ENSG00000157152 | 842 | NP_598328.1 | |
| SYN3 | ENSG00000185666 | 843 | NP_003481.3 | |
| SYNE1 | ENSG00000131018 | 844 | NP_892006.3 | X |
| SYT3 | ENSG00000213023 | 848 | NP_115674.1 | |
| TAF1 | ENSG00000147133 | 849 | NP_004597.2 | |
| TAF1C | ENSG00000103168 | 850 | NP_005670.3 | |
| TBC1D5 | ENSG00000131374 | 854 | NP_001127853.1 | |
| TBC1D7 | ENSG00000145979 | 855 | NP_001137436.1 | |
| TBL1X | ENSG00000101849 | 856 | NP_001132938.1 | |
| TBR1 | ENSG00000136535 | 858 | NP_006584.1 | |
| TCF20 | ENSG00000276461, | 860 | NP_005641.1 | |
| | ENSG00000262024, | 861 | | |
| | ENSG00000100207, | 862 | | |
| | ENSG00000280467, | 863 | | |
| | ENSG00000281897, | 864 | | |
| | ENSG00000283026, | 865 | | |
| | ENSG00000282892, | 866 | | |
| | ENSG00000283681 | 867 | | |
| TCF4 | ENSG00000196628 | 868 | NP_001230155.2 | |
| TCF7L2 | ENSG00000148737 | 869 | NP_001139746.1 | |
| TERT | ENSG00000164362 | 873 | NP_937983.2 | |
| TH | ENSG00000180176 | 876 | NP_954986.2 | |
| THRA | ENSG00000126351 | 879 | NP_003241.2 | |
| TOP3B | ENSG00000100038 | 889 | NP_003926.1 | X |
| TPO | ENSG00000115705, | 891 | NP_000538.3 | X |
| | ENSG00000277603 | 892 | | |
| TRIM33 | ENSG00000197323 | 893 | NP_056990.3 | X |
| TRIO | ENSG00000038382 | 894 | NP_009049.2 | |
| TRIP12 | ENSG00000153827 | 895 | NP_004229.1 | |
| TRPC6 | ENSG00000137672 | 896 | NP_004612.2 | X |
| TRPM1 | ENSG00000134160, | 897 | NP_001238949.1 | |
| | ENSG00000274965 | 898 | | |
| TSC1 | ENSG00000165699 | 899 | NP_000359.1 | |
| TSC2 | ENSG00000103197 | 900 | NP_000539.2 | X |
| TSN | ENSG00000211460 | 902 | NP_004613.1 | |
| TTN | ENSG00000155657 | 905 | NP_001254479.1 | X |
| TUBGCP5 | ENSG00000280807, | 906 | NP_001096080.1 | |
| | ENSG00000275835, | 907 | | |
| | ENSG00000276856 | 908 | | |
| UBE3A | ENSG00000114062 | 912 | NP_000453.2 | |
| UBE3B | ENSG00000151148 | 913 | NP_904324.1 | X |
| UBE3C | ENSG00000009335 | 914 | NP_055486.2 | |
| UPB1 | ENSG00000100024 | 922 | NP_057411.1 | |
| UPF3B | ENSG00000125351 | 924 | NP_542199.1 | |
| USP45 | ENSG00000123552 | 925 | NP_001073950.1 | |
| USP9Y | ENSG00000114374 | 927 | NP_004645.2 | |
| VIP | ENSG00000146469 | 931 | NP_003372.1 | |
| VLDLR | ENSG00000147852 | 932 | NP_003374.3 | X |
| WAC | ENSG00000095787 | 935 | NP_057712.2 | X |
| WDFY3 | ENSG00000163625 | 936 | NP_055806.2 | X |
| WNK3 | ENSG00000196632 | 938 | NP_065973.2 | X |
| WWOX | ENSG00000186153 | 941 | NP_057457.1 | X |
| ZBTB20 | ENSG00000181722 | 950 | NP_001157814.1 | X |
| ZMYND11 | ENSG00000015171 | 952 | NP_006615.2 | |
| ZNF18 | ENSG00000154957 | 953 | NP_653281.2 | |

*Representative protein in the family encoded by the gene.

TABLE 3
(152 Genes)
SFARI genes that have a very high scoring putative hnRNP L-binding
motif within 500 bp of one of the Castle splice sites.

| | | | | Genes with hnRNP |
|---|---|---|---|---|
| | | | | binding sites within |
| | | | | 200 base pairs of an |
| | | SEQ | Accession | RBFox1/A2BP1 |
| Human Gene | Ensembl Identifier | ID NO | Number* | binding site |
| A2BP1 | ENSG00000078328 | 967 | NP_665898.1 | X |
| ABCA10 | ENSG00000154263 | 2 | NP_525021.3 | |
| ACHE | ENSG00000087085 | 5 | NP_000656.1 | |
| ADAMTS18 | ENSG00000140873 | 10 | NP_955387.1 | |
| ADARB1 | ENSG00000197381 | 11 | NP_056648.1 | |
| ADNP | ENSG00000101126 | 14 | NP_056154.1 | X |
| AHI1 | ENSG00000135541 | 26 | NP_001128302.1 | |
| ANK3 | ENSG00000151150 | 35 | NP_066267.2 | |
| ANKRD11 | ENSG00000167522 | 36 | NP_001243111.1 | |
| ANXA1 | ENSG00000135046 | 38 | NP_000691.1 | |
| ARHGEF9 | ENSG00000131089 | 51 | NP_056000.1 | |
| ARID1B | ENSG00000049618 | 52 | NP_065783.3 | |
| ARNT2 | ENSG00000172379 | 53 | NP_055677.3 | |
| ASMT | ENSG00000196433 | 56 | NP_004034.2 | |
| ATP2B2 | ENSG00000157087 | 63 | NP_001001331.1 | |
| ATRNL1 | ENSG00000107518 | 65 | NP_997186.1 | X |
| ATRX | ENSG00000085224 | 66 | NP_000480.3 | |
| AUTS2 | ENSG00000158321 | 68 | NP_056385.1 | |
| BCAS1 | ENSG00000064787 | 74 | NP_003648.2 | X |
| BCKDK | ENSG00000103507 | 75 | NP_005872.2 | |
| BIN1 | ENSG00000136717 | 79 | NP_647593.1 | X |
| CACNA1B | ENSG00000148408 | 97 | NP_000709.1 | |
| CACNA1C | ENSG00000151067 | 98 | NP_955630.3 | |
| CACNA1E | ENSG00000198216 | 100 | NP_001192222.1 | |
| CACNA1F | ENSG00000102001 | 101 | NP_005174.2 | |
| CACNA1G | ENSG00000006283 | 102 | NP_061496.2 | |
| CAMTA1 | ENSG00000171735 | 113 | NP_056030.1 | X |
| CD38 | ENSG00000004468 | 124 | NP_001766.2 | |
| CD99L2 | ENSG00000102181 | 126 | NP_001229543.1 | |
| CHRNA7 | ENSG00000175344 | 146 | NP_001177384.1 | |
| CNKSR2 | ENSG00000149970 | 155 | NP_055742.2 | X |
| CNTN5 | ENSG00000149972 | 170 | NP_001230199.1 | |
| CNTNAP3 | ENSG00000106714 | 174 | NP_387504.2 | |
| CRHR2 | ENSG00000106113 | 178 | NP_001189404.1 | |
| DAPP1 | ENSG00000070190 | 198 | NP_055210.2 | |
| DDC | ENSG00000132437 | 202 | NP_001076440.1 | |
| DEAF1 | ENSG00000177030, | 206 | NP_066288.2 | |
| | ENSG00000282712 | 207 | | |
| DLG1 | ENSG00000075711 | 217 | NP_004078.2 | |
| DMD | ENSG00000198947 | 228 | NP_003997.1 | |
| DMXL2 | ENSG00000104093 | 230 | NP_001167587.1 | |
| DOCK4 | ENSG00000128512 | 239 | NP_055520.3 | |
| DOCK8 | ENSG00000107099 | 240 | NP_982272.2 | |
| DPP4 | ENSG00000197635 | 243 | NP_001926.2 | X |
| DRD2 | ENSG00000149295 | 248 | NP_000786.1 | |
| DUSP15 | ENSG00000149599 | 252 | NP_542178.2 | X |
| ELAVL2 | ENSG00000107105 | 266 | NP_004423.2 | |
| ELP4 | ENSG00000109911 | 268 | NP_061913.3 | |
| EPHB2 | ENSG00000133216 | 275 | NP_004433.2 | |
| ESR1 | ENSG00000091831 | 283 | NP_001116212.1 | |
| ESR2 | ENSG00000140009 | 284 | NP_001428.1 | |
| FMR1 | ENSG00000102081 | 312 | NP_002015.1 | |
| FOLH1 | ENSG00000086205 | 313 | NP_004467.1 | |
| FOXP1 | ENSG00000114861 | 315 | NP_001231739.1 | |
| FOXP2 | ENSG00000128573 | 316 | NP_683696.2 | |
| FRK | ENSG00000111816 | 317 | NP_002022.1 | |
| GABRB3 | ENSG00000166206 | 324 | NP_068712.1 | |
| GDA | ENSG00000119125 | 334 | NP_001229434.1 | X |
| GNAS | ENSG00000087460 | 341 | NP_536350.2 | |
| GTF2I | ENSG00000263001 | 371 | NP_127492.1 | |
| HDAC6 | ENSG00000094631 | 377 | NP_006035.2 | |
| HLA-DRB1 | ENSG00000236884, | 399 | NP_001230894.1 | |
| | ENSG00000228080, | 400 | | |
| | ENSG00000206240, | 401 | | |
| | ENSG00000229074, | 402 | | |
| | ENSG00000206306, | 403 | | |
| | ENSG00000196126 | 404 | | |
| HTR3A | ENSG00000166736 | 416 | NP_998786.2 | X |
| HTR7 | ENSG00000148680 | 418 | NP_062873.1 | |
| ICA1 | ENSG00000003147 | 422 | NP_071682.1 | |
| IQSEC2 | ENSG00000124313 | 437 | NP_001104595.1 | |
| ITPR1 | ENSG00000150995 | 442 | NP_001161744.1 | X |
| JMJD1C | ENSG00000171988 | 445 | NP_116165.1 | |
| KCNT1 | ENSG00000107147 | 459 | NP_065873.2 | |
| KHDRBS3 | ENSG00000131773 | 466 | NP_006549.1 | |
| KIF13B | ENSG00000197892 | 469 | NP_056069.2 | X |
| KIF5C | ENSG00000276734, | 470 | NP_004513.1 | |
| | ENSG00000168280 | 471 | | |
| KLC2 | ENSG00000174996 | 474 | NP_073733.1 | X |
| KMO | ENSG00000117009 | 475 | NP_003670.2 | X |
| LIN7B | ENSG00000104863 | 486 | NP_071448.1 | |
| LPL | ENSG00000175445 | 488 | NP_000228.1 | X |
| LRFN5 | ENSG00000165379 | 490 | NP_689660.2 | |
| LRRC7 | ENSG00000033122 | 495 | NP_065845.1 | X |
| MAP2 | ENSG00000078018 | 504 | NP_002365.3 | X |
| MARK1 | ENSG00000116141 | 509 | NP_061120.3 | |
| MBD1 | ENSG00000141644 | 510 | NP_001191065.1 | |
| MBD5 | ENSG00000204406 | 513 | NP_060798.2 | |
| MBD6 | ENSG00000166987 | 514 | NP_443129.3 | |
| MCC | ENSG00000171444 | 526 | NP_001078846.1 | X |
| MED12 | ENSG00000184634 | 531 | NP_005111.2 | |
| MEF2C | ENSG00000081189 | 533 | NP_001180276.1 | X |
| MET | ENSG00000105976 | 534 | NP_001120972.1 | |
| MNT | ENSG00000070444 | 538 | NP_064706.1 | |
| MSR1 | ENSG00000038945 | 543 | NP_619729.1 | |
| NBEA | ENSG00000172915 | 560 | NP_056493.3 | |
| NFIA | ENSG00000162599 | 572 | NP_001138984.1 | X |
| NIPA2 | ENSG00000140157 | 576 | NP_001171818.1 | |
| NLGN3 | ENSG00000196338 | 581 | NP_851820.1 | |
| NLGN4Y | ENSG00000165246 | 583 | NP_055708.3 | |
| NPAS2 | ENSG00000170485 | 587 | NP_002509.2 | |
| NRCAM | ENSG00000091129 | 590 | NP_001032209.1 | |
| NRG1 | ENSG00000157168 | 591 | NP_039250.2 | X |
| NRXN1 | ENSG00000179915 | 593 | NP_001129131.1 | X |
| NRXN3 | ENSG00000021645 | 595 | NP_004787.2 | |
| NTRK3 | ENSG00000140538 | 599 | NP_001012338.1 | X |
| OGT | ENSG00000147162 | 606 | NP_858058.1 | |
| P4HA2 | ENSG00000072682 | 618 | NP_001136071.1 | |
| PAH | ENSG00000171759 | 621 | NP_000268.1 | |
| PAX6 | ENSG00000007372 | 625 | NP_001245391.1 | X |
| PLXNB1 | ENSG00000164050 | 671 | NP_001123554.1 | |
| PSD3 | ENSG00000156011 | 691 | NP_056125.3 | |
| PTBP2 | ENSG00000117569 | 694 | NP_067013.1 | X |
| PTGER3 | ENSG00000050628 | 697 | NP_942011.1 | X |
| PTK7 | ENSG00000112655 | 699 | NP_001257327.1 | |
| PTPRC | ENSG00000262418, | 702 | NP_002829.3 | X |
| | ENSG00000081237 | 703 | | |
| RBM8A | ENSG00000265241 | 722 | NP_005096.1 | |
| RERE | ENSG00000142599 | 726 | NP_036234.3 | |
| RGS7 | ENSG00000182901 | 728 | NP_002915.3 | |
| RIMS1 | ENSG00000079841 | 730 | NP_055804.2 | |
| RNPS1 | ENSG00000205937 | 735 | NP_542161.1 | |
| RPL10 | ENSG00000147403 | 739 | NP_001243506.2 | |
| SAE1 | ENSG00000142230 | 743 | NP_005491.1 | |
| SCFD2 | ENSG00000184178 | 746 | NP_689753.2 | |
| SCN9A | ENSG00000169432 | 753 | NP_002968.1 | |
| SDK1 | ENSG00000146555 | 755 | NP_689957.3 | |
| SHANK2 | ENSG00000162105 | 768 | NP_036441.2 | |
| SIN3A | ENSG00000169375 | 773 | NP_001138829.1 | X |
| SLC24A2 | ENSG00000155886 | 781 | NP_065077.1 | X |
| SLC25A14 | ENSG00000102078 | 783 | NP_003942.1 | |
| SLC33A1 | ENSG00000169359 | 791 | NP_001177921.1 | X |
| SLC7A3 | ENSG00000165349 | 802 | NP_116192.4 | |
| SMARCA2 | ENSG00000080503 | 809 | NP_003061.3 | |
| SNRPN | ENSG00000128739 | 815 | NP_073716.1 | X |
| SPAST | ENSG00000021574 | 824 | NP_055761.2 | |
| SRGAP3 | ENSG00000196220 | 827 | NP_055665.1 | |
| ST7 | ENSG00000004866 | 830 | NP_068708.1 | |
| STXBP5 | ENSG00000164506 | 837 | NP_001121187.1 | |
| SYN3 | ENSG00000185666 | 843 | NP_003481.3 | |
| SYNE1 | ENSG00000131018 | 844 | NP_892006.3 | |
| TAF1 | ENSG00000147133 | 849 | NP_004597.2 | |
| TAF1C | ENSG00000103168 | 850 | NP_005670.3 | |
| TBL1X | ENSG00000101849 | 856 | NP_001132938.1 | |
| TCF20 | ENSG00000276461, | 860 | NP_005641.1 | |
| | ENSG00000262024, | 861 | | |
| | ENSG00000100207, | 862 | | |
| | ENSG00000280467, | 863 | | |
| | ENSG00000281897, | 864 | | |
| | ENSG00000283026, | 865 | | |
| | ENSG00000282892, | 866 | | |
| | ENSG00000283681 | 867 | | |
| TERT | ENSG00000164362 | 873 | NP_937983.2 | |
| THRA | ENSG00000126351 | 879 | NP_003241.2 | |
| TOP3B | ENSG00000100038 | 889 | NP_003926.1 | X |
| TPO | ENSG00000115705, | 891 | NP_000538.3 | |
| | ENSG00000277603 | 892 | | |
| TSC2 | ENSG00000103197 | 900 | NP_000539.2 | |
| TTN | ENSG00000155657 | 905 | NP_001254479.1 | |
| UBE3B | ENSG00000151148 | 913 | NP_904324.1 | |
| UPF3B | ENSG00000125351 | 924 | NP_542199.1 | |
| USP45 | ENSG00000123552 | 925 | NP_001073950.1 | |
| VIP | ENSG00000146469 | 931 | NP_003372.1 | |
| WAC | ENSG00000095787 | 935 | NP_057712.2 | |
| WWOX | ENSG00000186153 | 941 | NP_057457.1 | X |
| ZBTB20 | ENSG00000181722 | 950 | NP_001157814.1 | |
| ZMYND11 | ENSG00000015171 | 952 | NP_006615.2 | |
| ZNF18 | ENSG00000154957 | 953 | NP_653281.2 | |

*Representative protein in the family encoded by the gene.

TABLE 4
(78 Genes)
SFARI gene list with hnRNP L binding sites near splice
events specifically observed in autism.

| | | | | Genes with hnRNP |
|---|---|---|---|---|
| | | | | binding sites within |
| | | | | 200 base pairs of an |
| | | SEQ | Accession | RBFox1/A2BP1 |
| Human Gene | Ensembl Identifier | ID NO | Number* | binding site |
| ADCY5 | ENSG00000173175 | 12 | NP_899200.1 | |
| ANK2 | ENSG00000145362 | 34 | NP_001139.3 | |
| ANKRD11 | ENSG00000167522 | 36 | NP_001243111.1 | |
| ANKS1B | ENSG00000185046 | 37 | NP_690001.3 | X |
| APC | ENSG00000134982 | 42 | NP_001120982.1 | |
| ASTN2 | ENSG00000148219 | 58 | NP_054729.3 | |
| ATG7 | ENSG00000197548 | 60 | NP_006386.1 | |
| ATXN7 | ENSG00000163635 | 67 | NP_001170858.1 | X |
| BAIAP2 | ENSG00000175866 | 72 | NP_059345.1 | |
| BCAS1 | ENSG00000064787 | 74 | NP_003648.2 | X |
| CACNA1C | ENSG00000151067 | 98 | NP_955630.2 | |
| CACNA1E | ENSG00000198216 | 100 | NP_001192222.1 | |
| CACNA1G | ENSG00000006283 | 102 | NP_061496.2 | |
| CADM1 | ENSG00000182985 | 107 | NP_055148.3 | |
| CADPS2 | ENSG00000081803 | 109 | NP_001161412.1 | |
| CAMSAP2 | ENSG00000118200 | 112 | NP_982284.1 | |
| CAMTA1 | ENSG00000171735 | 113 | NP_056030.1 | X |
| CASC4 | ENSG00000166734 | 116 | NP_612432.2 | |
| CSMD1 | ENSG00000183117 | 179 | NP_150094.5 | X |
| CSNK1D | ENSG00000141551 | 180 | NP_001884.2 | |
| CUL7 | ENSG00000044090 | 187 | NP_001161842.1 | |
| DAPK1 | ENSG00000196730 | 197 | NP_004929.2 | |
| DST | ENSG00000151914 | 251 | NP_056363.2 | |
| ERBB4 | ENSG00000178568 | 279 | NP_005226.1 | X |
| GNAS | ENSG00000087460 | 341 | NP_536350.2 | X |
| GRIN1 | ENSG00000176884 | 358 | NP_001172019.1 | X |
| GSN | ENSG00000148180 | 369 | NP_000168.1 | |
| GTF2I | ENSG00000263001 | 371 | NP_127492.1 | |
| HDAC3 | ENSG00000171720 | 375 | NP_003874.2 | |
| HMGN1 | ENSG00000205581 | 405 | NP_004956.5 | |
| HUWE1 | ENSG00000086758 | 419 | NP_113584.3 | |
| KCNMA1 | ENSG00000156113 | 455 | NP_001154824.1 | |
| KCNQ2 | ENSG00000281151, | 456 | NP_742105.1 | |
| | ENSG00000075043 | 457 | | |
| KMT2A | ENSG00000118058 | 476 | NP_005924.2 | X |
| LAMB1 | ENSG00000091136 | 483 | NP_002282.2 | X |
| MAOB | ENSG00000069535 | 503 | NP_000889.3 | X |
| MAP2 | ENSG00000078018 | 504 | NP_002365.3 | X |
| MARK1 | ENSG00000116141 | 509 | NP_061120.3 | |
| MBD1 | ENSG00000141644 | 510 | NP_001191065.1 | X |
| MBD6 | ENSG00000166987 | 514 | NP_443129.3 | |
| MEF2C | ENSG00000081189 | 533 | NP_001180276.1 | X |
| MPP6 | ENSG00000105926 | 540 | NP_057531.2 | |
| MTOR | ENSG00000198793 | 546 | NP_004949.1 | |
| MYO9B | ENSG00000099331 | 553 | NP_004136.2 | X |
| NELL1 | ENSG00000165973 | 569 | NP_006148.2 | |
| NEO1 | ENSG00000067141 | 570 | NP_002490.2 | X |
| NFIX | ENSG00000008441 | 573 | NP_001257972.1 | X |
| NPAS2 | ENSG00000170485 | 587 | NP_002509.2 | |
| NRCAM | ENSG00000091129 | 590 | NP_001032209.1 | X |
| NRXN1 | ENSG00000179915 | 593 | NP_001129131.1 | X |
| NRXN3 | ENSG00000021645 | 595 | NP_004787.2 | X |
| OGT | ENSG00000147162 | 606 | NP_858058.1 | |
| P4HA2 | ENSG00000072682 | 618 | NP_001136071.1 | |
| PAFAH1B1 | ENSG00000007168 | 620 | NP_000421.1 | |
| PER1 | ENSG00000179094 | 654 | NP_002607.2 | X |
| PRKD1 | ENSG00000184304 | 686 | NP_002733.2 | |
| PRUNE2 | ENSG00000106772 | 690 | NP_056040.2 | |
| RAPGEF4 | ENSG00000091428 | 716 | NP_008954.2 | |
| RBFOX1/ | ENSG00000078328 | 720 | NP_665898.1 | |
| A2BP1 | | | | |
| RFWD2 | ENSG00000143207 | 727 | NP_071902.2 | X |
| RGS7 | ENSG00000182901 | 728 | NP_002915.3 | X |
| RIMS1 | ENSG00000079841 | 730 | NP_055804.2 | X |
| SCN1A | ENSG00000144285 | 747 | NP_001159435.1 | |
| SCN8A | ENSG00000196876 | 752 | NP_055006.1 | |
| SETD5 | ENSG00000168137 | 760 | NP_001073986.1 | |
| SGSH | ENSG00000181523 | 764 | NP_000190.1 | X |
| SMARCC2 | ENSG00000139613 | 810 | NP_003066.2 | |
| SNAP25 | ENSG00000132639 | 813 | NP_003072.2 | |
| ST7 | ENSG00000004866 | 830 | NP_068708.1 | X |
| SYNE1 | ENSG00000131018 | 844 | NP_892006.3 | |
| TBC1D5 | ENSG00000131374 | 854 | NP_001127853.1 | |
| TBC1D7 | ENSG00000145979 | 855 | NP_001137436.1 | |
| TCF4 | ENSG00000196628 | 868 | NP_001230155.1 | |
| TLK2 | ENSG00000146872 | 880 | NP_006843.2 | |
| TRIM33 | ENSG00000197323 | 893 | NP_056990.3 | |
| TSC2 | ENSG00000103197 | 900 | NP_000539.2 | |
| WDFY3 | ENSG00000163625 | 936 | NP_055806.2 | |
| ZBTB16 | ENSG00000109906 | 949 | NP_005997.2 | |

*Representative protein in the family encoded by the gene.

TABLE 5
(27 Genes)
Genes bearing the hnRNP L binding site, that comprise the SHANK-TSC-
mTOR-ERK ASD-associated pathways (pathways described in Goldani et al.,
Biomarkers in autism. Front Psychiatry. 2014 Aug. 12; 5: 100).

| Gene symbol | Table # | Name in Ref Figures | Patient cohort | Comment |
|---|---|---|---|---|
| NRXN1 | 2, 3, 4 | Neurexin, NRX | | |
| NRXN2 | 2 | Neurexin, NRX | | Established |
| | | | | target of |
| | | | | hnRNP L (ii) |
| NRXN3 | 2, 3, 4 | Neurexin, NRX | | |
| NLGN3 | 2, 3 | Neuroligin, NLGN | | |
| NLGN4X | 2 | Neuroligin, NLGN | | |
| NLGN4Y | 2, 3 | Neuroligin, NLGN | | |
| SHANK2 | 2, 3 | SHANK | | |
| SHANK3 | 2 | SHANK | | |
| NF1 | 2 | NF1 | Neurofibromatosis | Multiple splice |
| | | | | site mutations |
| TSC1 | 2 | TSC1 | Tuberous sclerosis | Multiple splice |
| | | | | site mutations |
| TSC2 | 2, 3, 4 | TSC2 | Tuberous sclerosis | Multiple splice |
| | | | | site mutations |
| MTOR | 4 | mTOR | | |
| FMR1 | 2, 3 | FMRP, FMR1 | (Fragile X syndrome) | |
| EIF4E | 2 | eiF4E | | |
| CACNA1C | 2, 3, 4 | CACNA1C | (Timothy syndrome) | |
| GRIN1 | 2, 4 | NMDAR | | |
| GRM1 | 2 | mGluR | | |
| DRD2 | 2, 3 | DRD2 | | |
| MAPK3 | 2 | ERK | | |
| GSK3B | 2 | GSK-3β | | |
| GABRB3 | 2, 3 | GABAR | | |
| SCN1A | 4 | Scn1A | | |
| MET | 2, 3 | MET | | |
| HRAS | 2 | Ras | | |
| VLDLR | 2 | VLDLR | | |
| AKAP9 | 2 | PKA | | |
| CADPS2 | 2, 4 | CADPS2 | | presynaptic |

TABLE 6
(18 Genes)
Genes bearing the hnRNP L binding site, that comprise
the SHANK-TSC- ASD disease module (i, ii).

| Gene symbol | Table # | Name in Ref Figures | Patient cohort | Comment |
|---|---|---|---|---|
| NRXN1 | 2, 3, 4 | Neurexin, NRX | | |
| NRXN2 | 2 | Neurexin, NRX | | Established |
| | | | | target of |
| | | | | hnRNP L (ii) |
| NRXN3 | 2, 3, 4 | Neurexin, NRX | | |
| NLGN3 | 2, 3 | Neuroligin, NLGN | | |
| NLGN4X | 2 | Neuroligin, NLGN | | |
| NLGN4Y | 2, 3 | Neuroligin, NLGN | | |
| SHANK2 | 2, 3 | SHANK | | |
| SHANK3 | 2 | SHANK | | |
| NF1 | 2 | NF1 | Neurofibromatosis | Multiple splice |
| | | | | site mutations |
| TSC1 | 2 | TSC1 | Tuberous sclerosis | Multiple splice |
| | | | | site mutations |
| TSC2 | 2, 3, 4 | TSC2 | Tuberous sclerosis | Multiple splice |
| | | | | site mutations |
| MTOR | 4 | mTOR | | |
| FMR1 | 2, 3 | FMRP, FMR1 | (Fragile X syndrome) | |
| EIF4E | 2 | eiF4E | | |
| CACNA1C | 2, 3, 4 | CACNA1C | (Timothy syndrome) | |
| GRIN1 | 2, 4 | NMDAR | | |
| GRM1 | 2 | mGluR | | |
| CADPS2 | 2, 4 | CADPS2, CAPS2 | | presynaptic |

i. Peça J., Feng G. Epub 2012 Mar. 20. Cellular and synaptic network defects in autism. Curr Opin Neurobiol. 2012 October; 22(5):866-72.
ii. Sadakata et al., Mouse models of mutations and variations in autism spectrum disorder-associated genes: mice expressing Caps2/Cadps2 copy number and alternative splicing variants. Int J Environ Res Public Health. 2013 Nov. 27; 10(12):6335-53.
iii. G. Rozic & Z. Lupowitz & N. Zisapel, Exonal Elements and Factors Involved in the Depolarization-Induced Alternative Splicing of Neurexin 2, J Mol Neurosci (2013) 50:221-233.
iv. Gauthier J, et al., Truncating mutations in NRXN2 and NRXN1 in autism spectrum disorders and schizophrenia. Hum Genet. 2011 October; 130(4):563-73.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### INCORPORATION BY REFERENCE

All of the cited U.S. patents, U.S. patent application publications and PCT patent applications designating the U.S., are hereby incorporated by reference in their entirety.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed.

1. A method of treating a subject with a neurological disease comprising identifying the subject comprising a splicing defect in an autism spectrum disorder (ASD)-associated gene, said target gene being characterized as having an hnRNP L binding site, and administering to said subject a spliceopathy rescue agent to repair said splicing defect, wherein said ASD-associated gene does not comprise AB11, ACSS2, AGAP3, AGXT2L2, APP, ATP2B1, ATP2B4, BIN1, BPTF/FALZ, C12orf41/KANSL2, C14orf133/VIPAR, DMD, DTNA, E1F2C2, EPB41L2, FMNL2, GARNL1/RALGAPA1, ITSN2, KIAA1217, LRRFIP1, MAPT, MAX, MEF2A, NCAM1, PALLD, PDLIM7, PPP2R5C, PTPN3, RPGR, RRN3, SAD1/BRSK2, SAMD4A, SEMA6D, SLC25A3, SLC39A9, SMTN, SORBS1, STXBP5, SVIL, TPM1, TPM3, TRIM66, TTN, VPS29, XPNPEP1, or ZMYND8.

2. The method of claim 1, wherein said subject is from a cohort with neurofibromatosis comprising a splicing defect in a NF1 gene or tuberous sclerosis comprising a splicing defect in a TSC1 or TSC2 gene.

3. The method of claim 1, wherein said splicing defect is in a target gene associated with ASD within the SHANK/TSC/mTOR/ERK signaling pathway.

4. The method of claim 3, wherein said target gene associated with ASD within the SHANK/TSC/mTOR signaling pathway is selected from the group consisting of CADPS2, NRXN1, NRXN2, NRXN3, NLGN3, NLGN4X, NLGN4Y, SHANK2, SHANK3, NF1, TSC1, TSC2, FMR1, EIF4E, CACNA1C, MTOR, GRIN1 and GRM1.

5. The method of claim 1, wherein said gene is characterized as having an hnRNP L binding site within the intron, or within the exon, adjacent to a site of alternative splicing.

6. The method of claim 1, wherein said gene is characterized as having an hnRNP L binding site within 500 base pairs of a site of alternative splicing.

7. The method of claim 1, wherein said gene further comprises an hnRNP binding site within 200 base pairs of an RBFox1/A2BP1 binding site.

8. The method of claim 1, wherein said gene further comprises an hnRNP binding site within 200 base pairs of the binding site of a splicing factor which is partner of hnRNP L in the splicing complex.

9. The method of claim 1, wherein said subject comprises a splice defect in an ASD-associated gene listed in Table 1, wherein said ASD-associated gene is a target of hnRNP L.

10. The method of claim 1, wherein said subject comprises a mutation in the target gene which results in spliceopathy.

11. The method of claim 1, wherein said splicing defect is in a gene selected from genes listed in Tables 2, 3, or 4.

12. The method of claim 1, wherein said splicing defect is in a gene selected from genes listed in Table 5.

13. The method of claim 1, wherein said splicing defect is in a gene selected from genes listed in Table 6.

14. The method of claim 1, wherein said subject comprises (a) social communication and social interactions characterized by deficits in social emotional reciprocity; deficits in non-verbal communication; and deficits in developing, maintaining and understanding relationships; and (b) restricted and repetitive behavior characterized by at least 2 of stereotyped movement or speech; insistence on sameness, routines, rituals; restricted, fixated interests; and atypical sensory reactivity.

15. A method of claim 1, wherein the spliceopathy rescue agent is one selected from a small molecule, a nucleic acid, an enzyme, a protein, a polypeptide, an antibody or a functional fragment thereof, an aptamer, a small interfering RNA, a microRNA, a small hairpin RNA, an antisense nucleic acid, and a PNA.

16. The method of claim 15, wherein said small molecule comprises ascochlorin, an ascochlorin derivative, or an ascochlorin analogue.

17. The method of claim 16, wherein said ascochlorin analogue comprises ascofuranone, an ascofuranone derivative or an ascofuranone analog.

18.-47. (canceled)

48. A method of identifying a subject suffering from or at risk of developing ASD or developing intellectual disability comprising
i) detecting a defect in an hnRNP L gene or mRNA or protein in a tissue or a cell of the subject;
ii) contacting a tissue or a bodily fluid sample from said subject with an hnRNP L binding agent and a detectable label to form a complex and measuring an amount of the complex; or
iii) detecting a defect in an ASD-associated gene or in the mRNA or protein of said gene in a tissue or a cell of the subject, said ASD-associated gene being characterized as having an hnRNP L binding site.

49.-55. (canceled)

56. A composition comprising a spliceopathy rescue agent for treating a subject with a neurological disease, wherein the composition repairs a splicing defect in an ASD-associated gene, wherein said target gene is characterized as having an hnRNP L binding site, and said ASD-associated gene does not comprise AB11, ACSS2, AGAP3, AGXT2L2, APP, ATP2B1, ATP2B4, BIN1, BPTF/FALZ, C12orf41/KANSL2, C14orf133/VIPAR, DMD, DTNA, ElF2C2, EPB41L2, FMNL2, GARNL1/RALGAPA1, ITSN2, KIAA1217, LRRFIP1, MAPT, MAX, MEF2A, NCAM1, PALLD, PDLIM7, PPP2R5C, PTPN3, RPGR, RRN3, SAD1/BRSK2, SAMD4A, SEMA6D, SLC25A3, SLC39A9, SMTN, SORBS1, STXBP5, SVIL, TPM1, TPM3, TRIM66, TTN, VPS29, XPNPEP1, or ZMYND8.

57.-79. (canceled)
